(19) United States
(12) Patent Application Publication
(10) Pub. No.: US 2022/0073896 A1
(43) Pub. Date: Mar. 10, 2022

(54) RECOMBINANT YEAST AND METHOD FOR PRODUCING ETHANOL USING SAME

(71) Applicant: TOYOTA JIDOSHA KABUSHIKI KAISHA, Toyota-shi, Aichi (JP)

(72) Inventors: Rie HIRAO, Handa-shi, Aichi (JP); Nobuki TADA, Nisshin-shi, Aichi (JP); Toru ONISHI, Toyota-shi, Aichi (JP)

(73) Assignee: TOYOTA JIDOSHA KABUSHIKI KAISHA, Toyota-shi, Aichi (JP)

(21) Appl. No.: 17/417,830

(22) PCT Filed: Dec. 24, 2019
(86) PCT No.: PCT/JP2019/050466

§ 371 Date: Jun. 24, 2021

(30) Foreign Application Priority Data
Dec. 25, 2018 (JP) 2018-241823

(51) Int. Cl. C12N 9/90; C12N 9/12; C12N 9/92; C12P 7/10

(52) U.S. Cl. CPC C12N 9/90; C12Y 503/01004; C12N 9/1205; C12P 7/10; C12Y 501/03004; C12N 9/92; C12Y 503/01005; C12Y 207/01016

(57) ABSTRACT

Provided are excellent L-arabinose metabolic genes that function in yeasts. Provided is an L-arabinose metabolic gene cluster including an L-arabinose isomerase gene specified by a predetermined SEQ ID, an L-ribulokinase gene specified by a predetermined SEQ ID, and an L-ribulose-5-phosphate-4-epimerase gene specified by a predetermined SEQ ID.

### BACKGROUND

### Technical Field

The present disclosure relates to a recombinant yeast having ethanol fermentation ability and a method for producing ethanol using the yeast.

### Background Art

Cellulosic biomass is effectively used as a raw material for useful alcohols such as ethanol and organic acids. In order to increase the amount of ethanol to be produced in the production of ethanol using cellulosic biomass, yeast strains that can use pentose such as D-xylose and L-arabinose as substrates have been developed. For example, a yeast strain having the ability to metabolize L-arabinose can be constructed by introducing a group of genes involved in the metabolism of L-arabinose into the yeast. Examples of the L-arabinose metabolic genes to be introduced into the yeast can include prokaryotic araA (L-arabinose isomerase), araB (L-ribulokinase), and araD (L-ribulose-5-phosphate-4-epimerase). Further, examples of the L-arabinose metabolic genes can include eukaryotic LXR (L-xylulose reductase) and LAD (L-L-arabinitol 4-dehydrogenase).

Non-Patent Literature 1 discloses a technique to produce ethanol from L-arabinose by introducing L-arabinose metabolic genes into a yeast. In particular, Non-Patent Literature 1 points out that the balance of coenzymes in the metabolic pathways of D-xylose and L-arabinose is poor in recombinant yeasts in which eukaryotic L-arabinose metabolic genes are introduced, and the conversion efficiency from L-arabinose into ethanol is poor as compared with recombinant yeasts in which prokaryotic L-arabinose metabolic genes are introduced.

Further, Non-Patent Literature 2 discloses a recombinant yeast in which the araA gene of Bacillus subtilis and the araB and araD genes of Escherichia coli are introduced as L-arabinose metabolic genes, and endogenous galactose permease (GAL2 gene) is overexpressed. Ethanol can be produced by using the recombinant yeast disclosed in Non-Patent Literature 2 to assimilate L-arabinose. It is known that the galactose permease encoded by a GAL2 gene is involved in the transportation of L-arabinose.

Further, Patent Literature 1 discloses that, in a recombinant yeast in which prokaryotic L-arabinose metabolic genes are introduced, the growth in an L-arabinose-containing medium is excellent, particularly, in the case where the araA gene is derived from Bacillus licheniformis or Clostridium acelobulylicum. In addition to this, Patent Literature 1 discloses that the growth in the L-arabinose-containing medium is superior in the case where the nucleotide sequences of at least two or more of the araA, araB, and araD genes are optimized for the codons of Saccharomyces cerevisiae.

Moreover, Non-Patent Literature 3 discloses a recombinant yeast (recombinant Saccharomyces cerevisiae) in which araA, araB, and araD genes derived from Lactobacillus plantarum are introduced. According to Non-Patent Literature 3, the recombinant yeast assimilates L-arabinose and produces ethanol in a medium containing L-arabinose as a single carbon source or a medium containing a mixed sugar containing L-arabinose as a carbon source under anaerobic conditions.

Further, Patent Literature 2 discloses a recombinant yeast in which araA, araB, and araD genes derived from Bacteroides thetaiotamicron are introduced. The recombinant yeast disclosed in Patent Literature 2 assimilates L-arabinose and produces ethanol. Further, Patent Literature 3 discloses a recombinant yeast in which araA, araB, and araD genes derived from Arthrobacter aurescens, araA, araB, and araD genes derived from Clavibacter michiganensis, or araA, araB, and araD genes derived from Gramella forseii are introduced.

### CITATION LIST

### Patent Literature

[Patent Literature 1] U.S. Pat. No. 8,753,862 B2
[Patent Literature 2] U.S. Pat. No. 9,598,689 B2
[Patent Literature 3] US 2010/0304454 A1

### Non-Patent Literature

[Non-Patent Literature 1] P. Richard et al., FEMS Yeast Research 3 (2003): 185-189
[Non-Patent Literature] Becker J, et al., Appl. Environ. Microbiol. (2003) July; 69(7): 4144-4150
[Non-Patent Literature] Wisselink, H. W. et al., Appl. Environ. Microbiol. (2007) 73: 4881-4891

### SUMMARY OF INVENTION

### Objects to be Attained by the Invention

However, the findings about the L-arabinose metabolic genes that function in yeasts are not sufficient, and the development of an excellent L-arabinose metabolic gene has been required. In view of the actual situation described above, the present disclosure provides a recombinant yeast that has acquired the ability to metabolize arabinose by finding out an excellent L-arabinose metabolic gene that functions, particularly, in yeasts and introducing the L-arabinose metabolic gene, and further provides a method for producing ethanol using the recombinant yeast.

### Means for Attaining the Objectives

As a result of diligent studies in order to provide the recombinant yeast and the method described above, the inventors have found a new L-arabinose metabolic gene that functions in yeasts, thereby accomplishing the present disclosure.

The present disclosure includes the following aspects.

(1) A recombinant yeast comprising a group of L-arabinose metabolic genes including an L-arabinose isomerase gene, an L-ribulokinase gene, and an L-ribulose-5-phosphate-4-epimerase gene introduced thereinto, wherein the L-arabinose isomerase gene is a gene encoding any one of proteins (a) to (c) below:
(a) a protein comprising one amino acid sequence selected from the group consisting of SEQ ID NOs: 2, 4, and 6;
(b) a protein comprising an amino acid sequence having an identity of 80% or more to one amino acid sequence selected from the group consisting of SEQ ID NOs: 2, 4, and 6 and having L-arabinose isomerase activity; and
(c) a protein encoded by a nucleotide sequence that hybridizes with a nucleotide sequence complementary to one nucleotide sequence selected from the group consisting of SEQ ID NOs: 1, 3, and 5 under stringent conditions and having L-arabinose isomerase activity.
(2) A recombinant yeast comprising a group of L-arabinose metabolic genes including an L-arabinose isomerase gene, an L-ribulokinase gene, and an L-ribulose-5-phosphate-4-epimerase gene introduced thereinto, wherein the L-ribulokinase gene is a gene encoding any one of proteins (a) to (c) below:
(a) a protein comprising one amino acid sequence selected from the group consisting of SEQ ID NOs: 8, 10, 12, 14, and 16;
(b) a protein comprising an amino acid sequence having an identity of 80% or more to one amino acid sequence selected from the group consisting of SEQ ID NOs: 8, 10, 12, 14, and 16 and having L-ribulokinase activity; and
(c) a protein encoded by a nucleotide sequence that hybridizes with a nucleotide sequence complementary to one nucleotide sequence selected from the group consisting of SEQ ID NOs: 7, 9, 11, 13, and 15 under stringent conditions and having L-ribulokinase activity.
(3) A recombinant yeast comprising a group of L-arabinose metabolic gene including an L-arabinose isomerase gene, an L-ribulokinase gene, and an L-ribulose-5-phosphate-4-epimerase gene introduced thereinto, wherein the L-ribulose-5-phosphate-4-epimerase gene is a gene encoding any one of proteins (a) to (c) below:
(a) a protein comprising one amino acid sequence selected from the group consisting of SEQ ID NOs: 18, 20, and 22;
(b) a protein comprising an amino acid sequence having an identity of 80% or more to one amino acid sequence selected from the group consisting of SEQ ID NOs: 18, 20, and 22 and having L-ribulose-5-phosphate-4-epimerase activity; and
(c) a protein encoded by a nucleotide sequence that hybridizes with a nucleotide sequence complementary to one nucleotide sequence selected from the group consisting of SEQ ID NOs: 17, 19, and 21 under stringent conditions and having L-ribulose-5-phosphate-4-epimerase activity.
(4) The recombinant yeast according to any one of (1) to (3), which overexpresses a galactose permease gene.
(5) The recombinant yeast according to (4), wherein the galactose permease gene is a gene encoding any one of proteins (a) to (c) below:
(a) a protein comprising the amino acid sequence of SEQ ID NO: 24;
(b) a protein comprising an amino acid sequence having an identity of 80% or more to the amino acid sequence of SEQ ID NO: 24 and having galactose permease activity; and
(c) a protein encoded by a nucleotide sequence that hybridizes with a nucleotide sequence complementary to the nucleotide sequence of SEQ ID NO: 23 under stringent conditions and having galactose permease activity.
(6) The recombinant yeast according to any one of (1) to (3), wherein a xylose isomerase gene is introduced.
(7) The recombinant yeast according to (6), wherein the xylose isomerase gene is a gene encoding any one of proteins (a) to (c) below:
(a) a protein comprising the amino acid sequence of SEQ ID NO: 26;
(b) a protein comprising an amino acid sequence having an identity of 80% or more to the amino acid sequence of SEQ ID NO: 26 and having xylose isomerase activity; and
(c) a protein encoded by a nucleotide sequence that hybridizes with a nucleotide sequence complementary to the nucleotide sequence of SEQ ID NO: 25 under stringent conditions and having xylose isomerase activity.
(8) A method for producing ethanol, comprising a step of culturing the recombinant yeast according to any one of (1) to (7) in a medium comprising arabinose for ethanol fermentation.
(9) The method for producing ethanol according to (8), wherein the medium comprises cellulose, and at least saccharification of the cellulose simultaneously proceeds with the ethanol fermentation.

The present specification includes the disclosure of Japanese Patent Application No. 2018-241823, based on which the present application claims the priority.

### Effects of Invention

Having the ability to metabolize L-arabinose, the recombinant yeast according to the present disclosure can be used for ethanol production using a medium comprising L-arabinose.

### DETAILED DESCRIPTION

Hereinafter, the present disclosure will be described further in detail with reference to Examples.

The recombinant yeast according to the present disclosure is a yeast that has acquired the ability to metabolize arabinose by introducing a group of L-arabinose metabolic gene including an L-arabinose isomerase gene (araA gene), an L-ribulokinase gene (araB gene), and an L-ribulose-5-phosphate-4-epimerase gene (araD gene). In the recombinant yeast according to the present disclosure, at least one of the araA, araB, and araD genes has the following feature. For example, the recombinant yeast according to the present disclosure may have the araA gene described below and conventionally known araB and araD genes. Further, the recombinant yeast according to the present disclosure may have the araB gene described below and conventionally known araA and araD genes, for example. The recombinant yeast according to the present disclosure may have the araD gene described below and conventionally known araA and araB genes. Alternatively, the recombinant yeast according to the present disclosure may have at least two of the araA, araB, and araD genes described below, and the rest may be a conventionally known gene. Further, the recombinant yeast according to the present disclosure may have the araA, araB, and araD genes described below.

<araA Gene>

The L-arabinose isomerase gene described herein is one gene selected from the group consisting of the araA gene of Bacillus licheniformis (NCBI Accession number: WP_011198012), the araA gene of Selenomonas ruminantium (NCBI Accession number: WP_072306024), and the araA gene of Lactobacillus sakei (NCBI Accession number: WP_011375537). These araA genes can impart the ability to metabolize arabinose to yeasts by being introduced into yeasts together with araB and araD genes.

However, the L-arabinose isomerase gene used in the recombinant yeast according to the present disclosure may be a gene that has a paralog relationship or homolog relationship in a narrow sense to such an araA gene.

Here, the amino acid sequences of L-arabinose isomerase encoded by the araA gene of Bacillus licheniformis, the araA gene of Selenomonas ruminantium, and the araA gene of Lactobacillus sakei are respectively represented by SEQ ID NOs: 2, 4, and 6. Further, the nucleotide sequences of the regions of the araA gene of Bacillus licheniformis, the araA gene of Selenomonas ruminantium, and the araA gene of Lactobacillus sakei encoding L-arabinose isomerase protein are respectively represented by SEQ ID NOs: 1, 3, and 5.

In addition, the L-arabinose isomerase gene used in the recombinant yeast according to the present disclosure is not limited to those encoding the amino acid sequences defined by these SEQ ID NOs and may be those encoding a protein comprising an amino acid sequence having an identity of 80% or more, 85% or more in some embodiments, 90% or more in other embodiments, 95% or more in still other embodiments, or 97% or more in some other embodiments, to one amino acid sequence selected from the group consisting of SEQ ID NOs: 2, 4, and 6 and having L-arabinose isomerase activity.

The identity values can be calculated by BLASTN or BLASTX programs that implement the BLAST algorithm (default setting). The identity values are calculated as a ratio of the number of amino acid residues that completely match each other when a pair of amino acid sequences are analyzed by pairwise alignment to the total amino acid residues compared.

Further, the L-arabinose isomerase gene is not limited to those specified by SEQ ID NOs: 1 to 6 and may be, for example, those encoding a protein having an amino acid sequence derived from the amino acid sequence of SEQ ID NO: 2, 4, or 6 by substitution, deletion, insertion or addition of one or several amino acids, and having L-arabinose isomerase activity. The number referred to as several herein means 2 to 50, 2 to 30 in some embodiments, 2 to 15 in other embodiments, or 2 to 7 in some other embodiments, for example.

Moreover, the L-arabinose isomerase gene is not limited to those specified by SEQ ID NOs: 1 to 6 and may be those hybridizing with the entire or a part of the complementary strand of DNA consisting of the nucleotide sequence of SEQ ID NO: 1, 3, or 5 under stringent conditions and encoding a protein having L-arabinose isomerase activity, for example. The term “stringent conditions” herein means conditions in which so-called specific hybrids are formed, and non-specific hybrids are not formed. The conditions can be appropriately determined with reference to Molecular Cloning: A Laboratory Manual (Third Edition), for example. Specifically, the stringency can be set by the temperature and the salt concentration contained in the solution in Southern hybridization, and the temperature and the salt concentration contained in the solution in the washing step of Southern hybridization. More specifically, the stringent conditions, for example, are a sodium concentration of 25 to 500 mM or 25 to 300 mM in some embodiments and a temperature of 42 to 68° C. or 42 to 65° C. in some embodiments. More specifically, the stringent conditions are 5×SSC (83 mM NaCl, 83 mM sodium citrate) and a temperature of 42° C.

As described above, whether or not a gene consisting of a nucleotide sequence different from SEQ ID NO: 1, 3, or 5 or a gene encoding an amino acid sequence different from SEQ ID NO: 2, 4, or 6 functions as an L-arabinose isomerase gene may be determined by producing an expression vector having such a gene incorporated into a site between a suitable promoter and a suitable terminator or the like, transforming a host such as Escherichia coli using the expression vector, and measuring the L-arabinose isomerase activity of a protein expressed. The L-arabinose isomerase activity is the activity to catalyze a reaction to produce L-ribulose using L-arabinose as a substrate. Accordingly, the L-arabinose isomerase activity can be evaluated based on the decrease in L-arabinose as a substrate and/or the increment in L-ribulose as a product, for example.

<araB Gene>

The L-ribulokinase gene described herein is one gene selected from the group consisting of the araB gene of Thermoactinomyces sp. (NCBI Accession number: WP_049720024), the araB gene of Clostridium nexile (NCBI Accession number: CDC22812), the araB gene of Selenomonas sp. oral taxon (NCBI Accession number: WP_050342034), the araB gene of Paenibacillus sp. (NCBI Accession number: WP_039877980), and the araB gene of Megasphaera cerevisiae (NCBI Accession number: WP_048515518). These araB genes can impart the ability to metabolize arabinose to yeasts by being introduced into the yeasts together with araA and araD genes.

However, the L-ribulokinase gene used in the recombinant yeast according to the present disclosure may be a gene that has a paralog relationship or homolog relationship in a narrow sense to such an araB gene.

Here, the amino acid sequences of L-ribulokinase encoded by the araB gene of Thermoactinomyces sp., the araB gene of Clostridium nexile, the araB gene of Selenomonas sp. oral taxon, the araB gene of Paenibacillus sp., and the araB gene of Megasphaera cerevisiae are respectively represented by SEQ ID NOs: 8, 10, 12, 14, and 16. Further, the nucleotide sequences of the regions of the araB gene of Thermoactinomyces sp., the araB gene of Clostridium nexile, the araB gene of Selenomonas sp. oral taxon, the araB gene of Paenibacillus sp., and the araB gene of Megasphaera cerevisiae encoding L-ribulokinase protein are respectively represented by SEQ ID NOs: 7, 9, 11, 13, and 15.

In addition, the L-ribulokinase gene used in the recombinant yeast according to the present disclosure is not limited to those encoding the amino acid sequences defined by these SEQ ID NOs and may be those encoding a protein comprising an amino acid sequence having an identity of 80% or more, 85% or more in some embodiments, 90% or more in other embodiments, 95% or more in still other embodiments, or 97% or more in some other embodiments, to one amino acid sequence selected from the group consisting of SEQ ID NOs: 8, 10, 12, 14, and 16 and having L-ribulokinase activity.

The identity values can be calculated by BLASTN or BLASTX programs that implement the BLAST algorithm (default setting). The identity values are calculated as a ratio of the number of amino acid residues that completely match each other when a pair of amino acid sequences are analyzed by pairwise alignment to the total amino acid residues compared.

Further, the L-ribulokinase gene is not limited to those specified by SEQ ID NOs: 7 to 16 and may be those encoding a protein having an amino acid sequence derived from the amino acid sequences of SEQ ID NOs: 8, 10, 12, 14, and 16 by substitution, deletion, insertion or addition of one or several amino acids, and having L-ribulokinase activity, for example. The number referred to as several herein is, for example, 2 to 50, 2 to 30 in some embodiments, 2 to 15 in other embodiments, or 2 to 7 in some other embodiments.

Moreover, the L-ribulokinase gene is not limited to those specified by SEQ ID NOs: 7 to 16 and may be those hybridizing with the entire or a part of the complementary strand of DNA consisting of the nucleotide sequence of SEQ ID NOs: 7, 9, 11, 13, and 15 under stringent conditions and encoding a protein having L-ribulokinase activity, for example. The term “stringent conditions” herein means conditions in which so-called specific hybrids are formed, and non-specific hybrids are not formed. The conditions can be appropriately determined with reference to Molecular Cloning: A Laboratory Manual (Third Edition), for example. Specifically, the stringency can be set by the temperature and the salt concentration contained in the solution in Southern hybridization, and the temperature and the salt concentration contained in the solution in the washing step of Southern hybridization. More specifically, the stringent conditions, for example, are a sodium concentration of 25 to 500 mM or 25 to 300 mM in some embodiments and a temperature of 42 to 68° C. or 42 to 65° C. in some embodiments. More specifically, the stringent conditions are 5×SSC (83 mM NaCl, 83 mM sodium citrate) and a temperature of 42° C.

As described above, whether or not a gene consisting of a nucleotide sequence different from SEQ ID NOs: 7, 9, 11, 13, and 15, or a gene encoding an amino acid sequence different from SEQ ID NO: 8, 10, 12, 14, and 16 functions as an L-ribulokinase gene may be determined by producing an expression vector with such a gene incorporated into a site between a suitable promoter and a suitable terminator or the like, transforming a host such as Escherichia coli using the expression vector, and measuring the L-ribulokinase activity of a protein expressed. The L-ribulokinase activity is the activity to catalyze a reaction to produce ADP and L-ribulose-5-phosphate using ATP and L-ribulose as substrates. Accordingly, the L-ribulokinase activity can be evaluated, for example, based on the decrease in ATP or L-ribulose as a substrate and/or the increment in ADP or L-ribulose-5-phosphate as a product.

<araD Gene>

The L-ribulose-5-phosphate-4-epimerase gene described herein is one gene selected from the group consisting of the araD gene of Bacillus licheniformis (NCBI Accession number: WP_003182291), the araD gene of Alkalibacterium putridalgicola (NCBI Accession number: WP_091486828), and the araD gene of Carnobacterium sp. 17-4 (NCBI Accession number: WP_013709965). These araD genes can impart the ability to metabolize arabinose to yeasts by being introduced into the yeasts together with araA and araB genes.

However, the L-ribulose-5-phosphate-4-epimerase gene used in the recombinant yeast according to the present disclosure may be a gene that has a paralog relationship or homolog relationship in a narrow sense to such an araD gene.

The amino acid sequences of L-ribulose-5-phosphate-4-epimerase encoded by the araD gene of Bacillus licheniformis, the araD gene of Alkalibacterium putridalgicola, and the araD gene of Carnobacterium sp. 17-4 are respectively represented by SEQ ID NOs: 18, 20, and 22. Further, the nucleotide sequences of the regions of the araD gene of Bacillus licheniformis, the araD gene of Alkalibacterium putridalgicola, and the araD gene of Carnobacterium sp. 17-4 encoding L-ribulose-5-phosphate-4-epimerase protein are respectively represented by SEQ ID NOs: 17, 19, and 21.

In addition, the L-ribulose-5-phosphate-4-epimerase gene used in the recombinant yeast according to the present disclosure is not limited to those encoding the amino acid sequences defined by these SEQ ID NOs and may be those encoding a protein having an amino acid sequence having an identity of 80% or more, 85% or more in some embodiments, 900% or more in other embodiments, 95% or more in still other embodiments, or 97% or more in some other embodiments, to one amino acid sequence selected from the group consisting of SEQ ID NOs: 18, 20, and 22, and having L-ribulose-5-phosphate-4-epimerase activity.

The identity values can be calculated by BLASTN or BLASTX programs that implement the BLAST algorithm (default setting). The identity values are calculated as a ratio of the number of amino acid residues that completely match each other when a pair of amino acid sequences are analyzed by pairwise alignment to the total amino acid residues compared.

Further, the L-ribulose-5-phosphate-4-epimerase gene is not limited to those specified by SEQ ID NOs: 17 to 22 and may be those encoding a protein having an amino acid sequence derived from the amino acid sequences of SEQ ID NOs: 18, 20, and 22 by substitution, deletion, insertion or addition of one or several amino acids, and having L-ribulose-5-phosphate-4-epimerase activity, for example. The number referred to as several herein is, for example, 2 to 50, 2 to 30 in some embodiments, 2 to 15 in other embodiments, or 2 to 7 in some other embodiments.

Moreover, the L-ribulose-5-phosphate-4-epimerase gene is not limited to those specified by SEQ ID NOs: 17 to 22 and may be those hybridizing with the entire or a part of the complementary strand of DNA consisting of the nucleotide sequence of SEQ ID NOs: 17, 19, and 21 under stringent conditions and encoding a protein having L-ribulose-5-phosphate-4-epimerase activity, for example. The term “stringent conditions” herein means conditions in which so-called specific hybrids are formed, and non-specific hybrids are not formed. The conditions can be appropriately determined with reference to Molecular Cloning: A Laboratory Manual (Third Edition), for example. Specifically, the stringency can be set by the temperature and the salt concentration contained in the solution in Southern hybridization, and the temperature and the salt concentration contained in the solution in the washing step of Southern hybridization. More specifically, the stringent conditions, for example, are a sodium concentration of 25 to 500 mM or 25 to 300 mM in some embodiments and a temperature of 42 to 68° C. or 42 to 65° C. in some embodiments. More specifically, the stringent conditions are 5×SSC (83 mM NaCl, 83 mM sodium citrate) and a temperature of 42° C.

As described above, whether or not a gene consisting of a nucleotide sequence different from SEQ ID NOs: 17, 19, and 21 or a gene encoding an amino acid sequence different from SEQ ID NOs: 18, 20, and 22 functions as an L-ribulose-5-phosphate-4-epimerase gene may be determined by producing an expression vector with such a gene incorporated into a site between a suitable promoter and a suitable terminator or the like, transforming a host such as Escherichia coli using the expression vector, and measuring the L-ribulose-5-phosphate-4-epimerase activity of a protein expressed. The L-ribulose-5-phosphate-4-epimerase activity is the activity to catalyze a reaction to produce D-xylulose-5-phosphate using L-ribulose-5-phosphate as a substrate. Accordingly, the L-ribulose-5-phosphate-4-epimerase activity can be evaluated based on the decrease in L-ribulose-5-phosphate as a substrate and/or the increment in D-xylulose-5-phosphate as a product, for example.

<Galactose Permease Gene>

The recombinant yeast according to the present disclosure may be those with galactose permease gene introduced so as to overexpress, in addition to the L-arabinose metabolism-related gene described above. The galactose permease gene encodes a protein that functions as a transporter of arabinose. Accordingly, the overexpression of galactose permease gene can improve the ability to incorporate arabinose into the recombinant yeast.

The term “overexpression of galactose permease gene” means that the expression level of the gene is made higher than that in a wild-type yeast by introducing an expression vector capable of expressing the gene or replacing an endogenous galactose permease gene promoter with a promoter for high expression. Alternatively, the term “overexpression of galactose permease gene” means to include introducing an exogenous galactose permease gene under the control of a promoter capable of inducing expression in a yeast.

The galactose permease gene of Saccharomyces cerevisiae is known as a GAL2 gene. The nucleotide sequence of the GAL2 gene of Saccharomyces cerevisiae and the amino acid sequence of a protein encoded by the GAL2 gene are respectively represented by SEQ ID NOs: 23 and 24.

However, the galactose permease gene used in the recombinant yeast according to the present disclosure may be a gene that has a paralog relationship or homolog relationship in a narrow sense to the GAL2 gene.

In addition, the galactose permease gene used in the recombinant yeast according to the present disclosure is not limited to those having the amino acid sequence of SEQ ID NO: 24 and may be those encoding a protein comprising an amino acid sequence having an identity of 80% or more, 85% or more in some embodiments, 90% or more in other embodiments, 95% or more in still other embodiments, or 97% or more in some other embodiments, to the amino acid sequence of SEQ ID NO: 24, and having galactose permease activity.

The identity values can be calculated by BLASTN or BLASTX programs that implement the BLAST algorithm (default setting). The identity values are calculated as a ratio of the number of amino acid residues that completely match each other when a pair of amino acid sequences are analyzed by pairwise alignment to the total amino acid residues compared.

Further, the galactose permease gene is not limited to those specified by SEQ ID NO: 24 and may be those encoding a protein having an amino acid sequence derived from the amino acid sequence of SEQ ID NO: 24 by substitution, deletion, insertion or addition or one or several amino acids, and having galactose permease activity, for example. The number referred to as several herein is, for example, 2 to 50, 2 to 30 in some embodiments, 2 to 15 in other embodiments, or 2 to 7 in some other embodiments.

Moreover, the galactose permease gene is not limited to those specified by SEQ ID NOs: 23 and 24 and may be those hybridizing with the entire or a part of the complementary strand of DNA consisting of the nucleotide sequence of SEQ ID NO: 23 under stringent conditions and encoding a protein having galactose permease activity, for example. The term “stringent conditions” herein means conditions in which so-called specific hybrids are formed, and non-specific hybrids are not formed. The conditions can be appropriately determined with reference to Molecular Cloning: A Laboratory Manual (Third Edition), for example. Specifically, the stringency can be set by the temperature and the salt concentration contained in the solution in Southern hybridization, and the temperature and the salt concentration contained in the solution in the washing step of Southern hybridization. More specifically, the stringent conditions, for example, are a sodium concentration of 25 to 500 mM or 25 to 300 mM in some embodiments and a temperature of 42 to 68° C. or 42 to 65° C. in some embodiments. More specifically, the stringent conditions are 5×SSC (83 mM NaCl, 83 mM sodium citrate) and a temperature of 42° C.

As described above, whether or not a gene consisting of a nucleotide sequence different from SEQ ID NO: 23 or a gene encoding an amino acid sequence different from SEQ ID NO: 24 functions as a galactose permease gene may be determined by producing an expression vector with such a gene incorporated into a site between a suitable promoter and a suitable terminator or the like, transforming a host such as Escherichia coli using the expression vector, and measuring the galactose permease activity of a protein to be expressed. The galactose permease activity is the activity to incorporate galactose and/or arabinose contained in the medium into cells. Accordingly, the galactose permease activity can be evaluated, for example, by culturing the transformed Escherichia coli described above in a medium containing galactose and/or arabinose based on the decrease in galactose and/or arabinose in the medium.

<Xylose Metabolism-Related Gene>

The recombinant yeast according to the present disclosure may further have a conventionally known xylose metabolism-related enzyme gene in addition to the L-arabinose metabolism-related gene so as to have the ability to metabolize xylose. Here, “having the ability to metabolize xylose” means both of: acquiring the ability to metabolize xylose by introducing a xylose metabolism-related enzyme gene into a yeast that does not originally have the ability to metabolize xylose; and inherently having the ability to metabolize xylose by having a xylose metabolism-related enzyme gene. More specifically, examples of the yeast having the ability to metabolize xylose can include a yeast that does not inherently have the ability to metabolize xylose, to which the ability to metabolize xylose has been imparted by introducing a xylose isomerase gene into the yeast, and a yeast to which the ability to metabolize xylose has been imparted by introducing other xylose metabolism-related genes.

The xylose isomerase gene (XI gene) is not particularly limited, and genes from any species may be used. For example, multiple xylose isomerase genes derived from termite intestinal protists, disclosed in JP 2011-147445 A, can be used without particular limitation. Further, examples of the xylose isomerase gene that can be used include genes derived from anaerobic fungi, Piromyces sp. type E2 (JP 2005-514951 T), anaerobic fungi, Cyllamyces aberensis, bacteria, Bacteroides thetaiotaomicron, bacteria, Clostridium phytofermentans, and Streptomyces murinus cluster.

Specifically, a xylose isomerase gene derived from Reticulitermes speratus intestinal protists is used as the xylose isomerase gene in some embodiments. The nucleotide sequence of the coding region of the xylose isomerase gene derived from Reticulitermes speratus intestinal protists and the amino acid sequence of the protein encoded by the gene are respectively represented by SEQ ID NOs: 25 and 26.

However, the xylose isomerase gene is not limited to those specified by SEQ ID NOs: 25 and 26 and may be a gene having a paralog relationship or a homologue relationship in a narrow sense, although the nucleotide sequence and the amino acid sequence are different.

Further, the xylose isomerase gene is not limited to those specified by SEQ ID NOs: 25 and 26 and may be those encoding a protein having an amino acid sequence having an identity of 70% or more, 80% or more in some embodiments, 90% or more in other embodiments, or 95% or more in some other embodiments, to the amino acid sequence of SEQ ID NO: 26 and having xylose isomerase activity, for example. The identity values can be calculated by BLASTN or BLASTX programs that implement the BLAST algorithm (default setting). The identity values are calculated as a ratio of the number of amino acid residues that completely match each other when a pair of amino acid sequences are analyzed by pairwise alignment to the total amino acid residues compared.

Further, the xylose isomerase gene is not limited to those specified by SEQ ID NOs: 25 and 26 and may be those encoding a protein having an amino acid sequence derived from the amino acid sequence of SEQ ID NO: 26 by substitution, deletion, insertion or addition of one or several amino acids, and having xylose isomerase activity, for example. The number referred to as several herein is, for example, 2 to 30, 2 to 20 in some embodiments, 2 to 10 in other embodiments, or 2 to 5 in some other embodiments.

Moreover, the xylose isomerase gene is not limited to those specified by SEQ ID NOs: 25 and 26 and may be those hybridizing with the entire or a part of the complementary strand of DNA consisting of the nucleotide sequence of SEQ ID NO: 25 under stringent conditions and encoding a protein having xylose isomerase activity, for example. The term “stringent conditions” herein means conditions in which so-called specific hybrids are formed, and non-specific hybrids are not formed. The conditions can be appropriately determined with reference to Molecular Cloning: A Laboratory Manual (Third Edition), for example. Specifically, the stringency can be set by the temperature and the salt concentration contained in the solution in Southern hybridization, and the temperature and the salt concentration contained in the solution in the washing step of Southern hybridization. More specifically, the stringent conditions, for example, are a sodium concentration of 25 to 500 mM or 25 to 300 mM in some embodiments and a temperature of 42 to 68° C. or 42 to 65° C. in some embodiments. More specifically, the stringent conditions are 5×SSC (83 mM NaCl, 83 mM sodium citrate) and a temperature of 42° C.

As described above, whether or not a gene consisting of a nucleotide sequence different from SEQ ID NO: 25 or a gene encoding an amino acid sequence different from SEQ ID NO: 26 functions as a xylose isomerase gene may be determined by producing an expression vector with such a gene incorporated into a site between a suitable promoter and a suitable terminator or the like, transforming a host such as Escherichia coli using the expression vector, and measuring the xylose isomerase activity of a protein expressed. The term “xylose isomerase activity” means the activity to isomerize xylose into xylulose. Therefore, the xylose isomerase activity can be evaluated by preparing a solution containing xylose as a substrate, allowing the protein as an inspection target to act at a suitable temperature, and measuring the decrease in xylose and/or the amount of xylulose produced.

In particular, the xylose isomerase gene to be used in some embodiments is a gene encoding mutant xylose isomerase consisting of an amino acid sequence with a specific mutation introduced into specific amino acid residues in the amino acid sequence represented by SEQ ID NO: 26 and having improved xylose isomerase activity. Specifically, examples of the gene encoding mutant xylose isomerase can include a gene encoding an amino acid sequence with a substitution of asparagine at position 337 in the amino acid sequence represented by SEQ TD NO: 26 with cysteine. The xylose isomerase consisting of the amino acid sequence with the substitution of the asparagine at position 337 in the amino acid sequence represented by SEQ ID NO: 26 with cysteine has excellent xylose isomerase activity as compared with wild-type xylose isomerase. The mutant xylose isomerase is not limited to those with the substitution of the asparagine at position 337 with cysteine and may be those with the substitution of the asparagine at position 337 with an amino acid other than cysteine, those with different substitutions, in addition to the asparagine at position 337, with another amino acid, or those with a substitution of an amino acid residue other than the asparagine at position 337.

Meanwhile, the term “xylose metabolism-related gene other than the xylose isomerase gene” means to include a xylose reductase gene encoding xylose reductase that converts xylose into xylitol, a xylitol dehydrogenase gene encoding xylitol dehydrogenase that converts xylitol into xylulose, and a xylulokinase gene encoding xylulokinase that produces xylulose 5-phosphate by phosphorylating xylulose. The xylulose 5-phosphate produced by xylulokinase enters the pentose phosphate pathway to be metabolized.

The xylose metabolism-related gene is not particularly limited, but examples thereof can include a xylose reductase gene and a xylitol dehydrogenase gene derived from Pichia stipitis, and a xylulokinase gene derived from Saccharomyces cerevisiae (see Eliasson A. et al., Appl. Environ. Microbiol, 66: 3381-3386 and Toivari M N et al., Metab. Eng. 3: 236-249). In addition, examples of the xylose reductase gene that can be used include xylose reductase genes derived from Candida tropicalis and Candida prapsilosis. Examples of the xylitol dehydrogenase gene that can be used include xylitol dehydrogenase genes derived from Candida tropicalis or Candida prapsilosis. Examples of the xylulokinase gene that can be used also include xylulokinase genes derived from Pichia stipitis.

Further, the yeast inherently having the ability to metabolize xylose is not particularly limited, but examples thereof can include Pichia stipitis, Candida tropicalis, and Candida prapsilosis.

<Other Genes>

Meanwhile, the recombinant yeast according to the present disclosure may be a yeast comprising still another gene introduced thereinto. For example, the recombinant yeast according to the present disclosure may be one comprising an acetaldehyde dehydrogenase gene introduced thereinto. The acetaldehyde dehydrogenase gene is not particularly limited, and genes of any organism may be used. Further, the acetaldehyde dehydrogenase gene uses a gene with a nucleotide sequence modified according to the codon usage frequency in the yeast to be introduced, in the case of using genes derived from organisms other than fungi such as yeasts, e.g., bacteria, animals, plants, insects, and algae, in some embodiments.

More specifically, examples of the acetaldehyde dehydrogenase gene that can be used include the mhpF gene of Escherichia coli and the ALDH1 gene of Entamoeba histolytica as disclosed in Applied and Environmental Microbiology, May 2004, p. 2892-2897, Vol. 70, No. 5. Further, examples of the acetaldehyde dehydrogenase gene can include the adhE gene of Escherichia coli, an acetaldehyde dehydrogenation gene derived from Clostridium beijerinckii, and an acetaldehyde dehydrogenation gene derived from Chlamydomonas reinhardtii.

Further, the recombinant yeast according to the present disclosure may be, for example, a yeast comprising a gene that is involved in glucose metabolism such as glucose introduced thereinto. As an example, the recombinant yeast can be made into a yeast having β-glucosidase activity by introducing a β-glucosidase gene.

Here, the term “β-glucosidase activity” means the activity to catalyze a reaction of hydrolyzing a β-glycosidic bond of a sugar. That is, β-glucosidase can decompose cellooligosaccharides such as cellobiose into glucose. The β-glucosidase gene can be introduced as a cell-surface display gene. Here, the cell-surface display gene is a gene modified so that the protein encoded by the gene is expressed so as to be displayed on the surface layer of a cell. For example, a cell-surface display a glucosidase gene is a gene in which a β-glucosidase gene and a cell-surface localized protein gene are fused. A cell-surface localized protein is a protein that is fixed on the cell surface of a yeast and exists on the cell surface. Examples thereof include α- or a-agglutinin and FLO protein, which are aggregated proteins. In general, the cell-surface localized protein has a secretory signal sequence on the N-terminal side and a GPI-anchored recognition signal on the C-terminal side. The cell-surface localized protein is similar to secretory proteins in that it has a secretory signal, but the cell-surface localized protein is different from secretory proteins in that it is fixed to a cell membrane via a GPI anchor and transported. The cell-surface localized protein is fixed to the cell membrane by selectively cleaving the GPT-anchored recognition signal sequence, when passing through the cell membrane, and binding to the GPI anchor at a newly protruding C terminal part. Thereafter, the basal portion of the GPI anchor is cleaved by phosphatidylinositol-dependent phospholipase C (PI-PLC). Then, the protein separated from the cell membrane is incorporated into the cell wall to be fixed to the cell surface layer and localized on the cell surface layer (for example, see JP 2006-174767 A).

The β-glucosidase gene is not particularly limited, but examples thereof can include a β-glucosidase gene derived from Aspergillus aculeatus (Murai et al., Appl. Environ. Microbiol. 64: 4857-4861). In addition, examples of the β-glucosidase gene that can be used include a β-glucosidase gene derived from Aspergillus oryzae, a β-glucosidase gene derived from Clostridium cellulovorans, and a β-glucosidase gene derived from Saccharomycopsis fibuligera.

Further, the recombinant yeast used in the method for producing ethanol according to the present disclosure may be one with a gene encoding another enzyme constituting cellulase introduced, in addition to the β glucosidase gene or other than the β glucosidase gene. Examples of the enzyme constituting cellulase other than the β glucosidase can include exo-type cellobiohydrolases (CBH1 and CBH2) that release cellobiose from the ends of crystalline cellulose, and an end-type endoglucanase (EG) that cannot decompose crystalline cellulose but cleaves non-crystalline cellulose (amorphous cellulose) chains at random.

Further, examples of other genes to be introduced into the recombinant yeast can include an alcohol dehydrogenase gene (ADH1 gene) having the activity to convert acetaldehyde into ethanol, an acetyl-CoA synthase gene (ACS1 gene) having the activity to convert acetic acid into acetyl-CoA, and genes (ALD4 gene, ALD5 gene, and ALD6 gene) having the activity to convert acetaldehyde into acetic acid. An alcohol dehydrogenase gene (ADH2 gene) having the activity to convert ethanol into acetaldehyde may be disrupted.

Further, the recombinant yeast according to the present disclosure may have a feature of expressing an alcohol dehydrogenase gene (ADH1 gene) having the activity to convert acetaldehyde into ethanol at a high level in some embodiments. Examples of the method for expressing the gene at a high level include a method of replacing the intrinsic promoter of the gene with a promoter for high expression and a method of introducing an expression vector capable of expressing the gene into the yeast.

Further, the recombinant yeast according to the present disclosure may have a feature of having a reduced expression level of the alcohol dehydrogenase gene (ADH2 gene) having the activity to convert ethanol into aldehyde in some embodiments. Examples of the method for reducing the expression level of the gene include a method of modifying the intrinsic promoter of the gene and a method of deleting the gene. When deleting the gene, one of the pair of ADH2 genes existing in the diploid recombinant yeast may be deleted, or both of them may be deleted. Examples of the technique for reducing the gene expression can include so-called transposon method, transgene method, post-transcriptional gene silencing method, RNAi method, nonsense mediated decay (NMD) method, ribozyme method, antisense method, miRNA (micro-RNA) method, and siRNA (small interfering RNA) method.

Further, examples of other genes to be introduced into the recombinant yeast can include genes that can promote the utilization of xylose in a medium. Specifically, examples thereof can include a gene encoding xylulokinase having the activity to produce xylulose-5-phosphate using xylulose as a substrate. The metabolic flux in the pentose phosphate pathway can be improved by introducing the xylulokinase gene.

Further, a gene encoding an enzyme selected from an enzyme group constituting the pathway of the non-oxidation process in the pentose phosphate pathway can be introduced into the recombinant yeast. Examples of the enzymes constituting the non-oxidation process in the pentose phosphate pathway can include ribose-5-phosphate isomerase, ribulose-5-phosphate-3-epimerase, transketolase, and transaldolase. One or more genes encoding these enzymes are introduced in some embodiments. Further, two or more of such genes are introduced in combination in some embodiments, three or more are introduced in combination in some other embodiments, or all of the genes are introduced in still other embodiments.

More specifically, the xylulokinase (XK) gene can be used without particular limitation of the origin thereof. Many microorganisms such as bacteria and yeasts that assimilate xylulose comprise the XK gene. Information on the XK gene can be appropriately obtained by searching the NCBI website or the like. In some embodiments, examples thereof include XK genes derived from yeasts, lactic acid bacteria, Escherichia coli, and plants. Examples of the XK gene include an XK gene derived from S. cerevisiae S288C strain, XKS1 (GenBank: Z72979) (nucleotide sequence and amino acid sequence of the coding region of CDS).

More specifically, the transaldolase (TAL) gene, the transketolase (TKL) gene, the ribulose-5-phosphate epimerase (RPE) gene, and the ribose-5-phosphate ketoisomerase (RKI) gene can be used as without particular limitation of the origin thereof. Many organisms that have the pentose phosphate pathway comprise these genes. For example, general-purpose yeasts such as S. cerevisiae also carry these genes. Information on these genes can be appropriately obtained by accessing a web site such as NCBI. In some embodiments, examples thereof include genes derived from the same genus as the host eukaryotic cell, such as an eukaryotic cell or a yeast, and the same species as the host eukaryotic cell in still other embodiments. In some embodiments, a TAL1 gene can be used as the TAL gene, a TKL1 gene and a TKL2 gene can be used as the TKL gene, an RPE1 gene can be used as the RPE gene, and an RKI1 gene can be used as the RKI gene. Examples of these genes include a TAL1 gene derived from S. cerevisiae S288 strain, TAL1 gene (GenBank: U19102), a TKL1 gene derived from S. cerevisiae S288 strain (GenBank: X73224), an RPE1 gene derived from S. cerevisiae S288 strain (GenBank: X83571), and a RKI gene derived from S. cerevisiae S288 strain (GenBank: Z75003).

<Production of Recombinant Yeast>

The recombinant yeast according to the present disclosure can be produced, for example, by introducing a group of L-arabinose metabolic genes including at least one selected from the group consisting of the L-arabinose isomerase gene (araA gene), the L-ribulokinase gene (araB gene), and the L-ribulose-5-phosphate-4-epimerase gene (araD gene) into a yeast as a host. Alternatively, the recombinant yeast according to the present disclosure can be produced, for example, by further introducing at least one selected from the group consisting of the L-arabinose isomerase gene (araA gene), the L-ribulokinase gene (araB gene), and the L-ribulose-5-phosphate-4-epimerase gene (araD gene) into a yeast having the ability to metabolize L-arabinose.

The galactose permease gene, the xylose metabolism-related gene, and other genes may be introduced into the recombinant yeast according to the present disclosure, or a modification to reduce the expression level of the alcohol dehydrogenase gene (ADH2 gene) having the activity to convert ethanol into aldehyde may be performed.

When introducing the L-arabinose metabolic gene cluster, the galactose permease gene, the xylose metabolism-related gene, and other genes into the host yeast in production of the recombinant yeast according to the present disclosure, all the genes may be introduced at the same time or may be sequentially introduced using different expression vectors.

The yeast that can be used as a host is not particularly limited, but examples thereof include yeasts of Candida shehatae, Pichia stipitis, Pachysolen tannophilus, Saccharomyces cerevisiae, and Schizosaccharomyces pombe. In particular, Saccharomyces cerevisiae is used in some embodiments. The yeast may be an experimental strain used for experimental convenience or an industrial strain (practical strain) used for practical usefulness. Examples of the industrial strain include yeast strains used for making wine, sake, and shochu.

As the host yeast, homothallic yeasts are used in some embodiments. According to the technique disclosed in JP 2009-34036 A, use of a yeast having homothallic properties conveniently enables multicopy transfection into a genome. The yeasts having homothallic properties is synonymous with homothallic yeasts. The yeasts having homothallic properties are not particularly limited, and any yeast can be used. Examples of the yeasts having homothallic properties are not particularly limited, but can include Saccharomyces cerevisiae OC-2 strain (NBRC2260). Examples of other yeasts having homothallic properties can include an alcohol yeast (Daiken 396 No., NBRC0216) (Source: “Characteristics of alcohol yeast” Shuken Kaihou (Bulletin), No37, p 18-22 (1998.8)), isolated in Brazil and Okinawa ethanol produce yeast (Source: “Genetic properties of wild strains of Saccharomyces cerevisiae isolated in Brazil and Okinawa” Journal of Japan Society for Bioscience, Biotechnology, and Agrochemistry, Vol. 65, No. 4, p 759-762 (1991.4)) and 180 (Source “The screening of yeast having strong alcoholic fermentation ability” Journal of Brewing Society of Japan, Vol. 82, No. 6, p 439-443 (1987.6)). Further, even yeasts showing a heterothallic phenotype can be used as yeasts having homothallic properties by introducing the HO gene so as to express. That is, the yeasts having homothallic properties in the present disclosure mean to include yeasts with the HO gene introduced so as to express.

Among these, the Saccharomyces cerevisiae OC-2 strain is used in some embodiments, since it is a strain that has been conventionally used for wine brewing and confirmed to be safe. Further, the Saccharomyces cerevisiae OC-2 strain is used in some embodiments, since it is a strain having excellent promoter activity under high sugar concentration conditions, as will be described in Examples below. In particular, the Saccharomyces cerevisiae OC-2 strain is used in some embodiments due to excellent promoter activity of a pyruvate decarboxylase gene (PDC1) under high sugar concentration conditions.

Further, the promoter of the gene to be introduced is not particularly limited, but examples thereof that can be used include the promoter of a glyceraldehyde 3 phosphate dehydrogenase gene (TDH3), the promoter of a 3-phosphoglycerate kinase gene (PGK1), and the promoter of a hyperosmotic response 7 gene (HOR7). Among these, the promoter of pyruvate decarboxylase gene (PDC1) is used in some embodiments due to its high ability to express the target gene downstream at a high level.

That is, the aforementioned gene may be introduced into the genome of the yeast together with a promoter that regulates the expression and other expression-regulating regions. Alternatively, the aforementioned gene may be introduced so that its expression is regulated by the promoter of the gene originally existing in the genome of the yeast serving as the host or other expression-regulating regions.

Further, as the method for introducing the aforementioned gene, any technique conventionally known as a transformation method of yeast can be applied. Specifically, examples thereof include the electroporation method “Meth. Enzym., 194, p 182 (1990)”, the spheroplast method “Proc. Natl. Acad. Sci. USA, 75 p 1929 (1978)”, the lithium acetate method “J. Bacteriology, 153, p 163 (1983)”, Proc. Natl. Acad. Sci. USA, 75 p 1929 (1978), and Methods in yeast genetics, 2000 Edition: A Cold Spring Harbor Laboratory Course Manual, but there is no limitation to these methods.

Examples of the method for reducing the expression level of the alcohol dehydrogenase gene (ADH2 gene) having the activity to convert ethanol into aldehyde include a method of modifying the intrinsic promoter of the gene and a method of deleting the gene. When deleting the gene, one of the pair of genes existing in the diploid recombinant yeast may be deleted, or both of them may be deleted. Examples of the technique for reducing the gene expression can include so-called transposon method, transgene method, post-transcriptional gene silencing method, RNAi method, nonsense mediated decay (NMD) method, ribozyme method, antisense method, miRNA (micro-RNA) method, and siRNA (small interfering RNA) method.

<Production of Ethanol>

When producing ethanol using the recombinant yeast described above, ethanol fermentation culture is performed in a medium containing at least arabinose. That is, the medium for ethanol fermentation contains at least arabinose as a carbon source. The medium may contain other carbon sources such as glucose and xylose in advance.

Further, the carbon sources such as arabinose contained in the medium used for ethanol fermentation can be derived from biomass. In other words, the medium used for ethanol fermentation may have a composition containing cellulosic biomass and hemicellulase that produces arabinose or the like by saccharifying the hemicellulose contained in the cellulosic biomass. Here, the cellulosic biomass may be subjected to a conventionally known pretreatment. The pretreatment is not particularly limited, but examples thereof can include treatment to decompose lignin by microorganisms and crushing treatment of cellulosic biomass. Further, examples of the pretreatment that may be applied include treatment to immerse the cellulosic biomass crushed in a dilute sulfuric acid solution, an alkali solution, or an ionic liquid, hydrothermal treatment, and pulverization treatment. These pretreatments can improve the saccharification rate of the biomass.

When producing ethanol using the recombinant yeast described above, the medium may have a composition further containing cellulose and cellulase. In this case, the medium contains glucose produced by cellulase acting on cellulose. In the case where the medium used for ethanol fermentation contains cellulose, the cellulose can be derived from biomass. In other words, the medium used for ethanol fermentation may have a composition containing cellulase capable of saccharifying cellulase contained in the cellulosic biomass.

Further, the medium used for ethanol fermentation may be supplemented with a saccharified solution after the saccharification treatment of the cellulosic biomass. In this case, the saccharified solution contains residual cellulose and glucose, and arabinose and xylose derived from the hemicellulose contained in the cellulosic biomass.

As described above, the method for producing ethanol according to the present disclosure comprises an ethanol fermentation step using at least arabinose as a sugar source. In the method for producing ethanol according to the present disclosure, ethanol can be produced by ethanol fermentation using arabinose as a sugar source. In the method for producing ethanol using the recombinant yeast according to the present disclosure, ethanol is recovered from the medium after the ethanol fermentation. The method for recovering ethanol is not particularly limited, and any conventionally known method can be applied. For example, after the ethanol fermentation has ended, a liquid layer containing ethanol and a solid layer containing the recombinant yeast and solid components are separated by solid-liquid separation operation. Thereafter, the ethanol contained in the liquid layer is separated and purified by a distillation method, so that high-purity ethanol can be recovered. The degree of purification of ethanol can be appropriately adjusted according to the purpose of use of ethanol.

Further, the method for producing ethanol according to the present disclosure may be a so-called simultaneous saccharification fermentation process, in which a step of saccharifying the cellulose contained in the medium by cellulase and a step of fermenting ethanol using arabinose and glucose produced by the saccharification as sugar sources proceed at the same time. Here, the term “simultaneous saccharification fermentation process” means a process in which a step of saccharifying cellulosic biomass and a step of fermenting ethanol are performed at the same time without distinguishing between them.

The saccharification method is not particularly limited, but examples thereof can include an enzymatic method using a cellulase preparation such as cellulase and hemicellulase. The cellulase preparation contains multiple enzymes that are involved in decomposition of cellulose chains and hemicellulose chains and exhibits multiple activities such as endoglucanase activity, endoxylanase activity, cellobiohydrolase activity, glucosidase activity, and xylosidase activity. The cellulase preparation is not particularly limited, but examples thereof can include cellulases produced by Trichoderma reesei and Acremonium cellulolyticus. A commercially available cellulase preparation also may be used.

In the simultaneous saccharification fermentation process, a cellulase preparation and the recombinant microorganisms are added to a medium containing cellulosic biomass (which may be pretreated), and the recombinant yeast is cultured in a predetermined temperature range. The culture temperature is not particularly limited but can be 25 to 45° C. or is 30 to 40° C. in consideration of the efficiency of ethanol fermentation in some embodiments. Further, the pH of the culture solution is 4 to 6 in some embodiments. Further, stirring or shaking may be performed in culture. Further, anomalous simultaneous saccharification fermentation may be carried out, in which saccharification is first carried out at the optimum temperature of the enzyme (40 to 70° C.), then the temperature is lowered to a predetermined temperature (30 to 40° C.), and the yeast is added.

Meanwhile, the recombinant yeast according to the present disclosure is excellent in the ability to metabolize arabinose, that is, the efficiency of assimilation of arabinose contained in the medium to produce ethanol. Accordingly, the recombinant yeast according to the present disclosure can produce ethanol by using not only glucose produced during saccharification of cellulosic biomass but also arabinose effectively, to improve the ethanol productivity from cellulosic biomass considerably.

### EXAMPLES

Hereinafter the present disclosure will be described further in detail by way of examples, but the technical scope of the present disclosure is not limited to the following examples.

### Example 1

In this example, a new L-arabinose isomerase gene (araA gene), a new L-ribulokinase gene (araB gene), and a new L-ribulose-5-phosphate-4-epimerase gene (araD gene) which contribute to assimilation of arabinose were searched for.

(1) Screening of araB Gene

A recombinant yeast was produced by introducing each of 11 types of new araB genes and known araA and araD genes derived from Lactobacillus plantarum into a yeast overexpressing a GAL2 gene derived from S. cerevisiae (encoding an arabinose transporter). Then, the recombinant yeast was compared with the case where a known araB gene derived from Lactobacillus plantarum was introduced for investigation, to find out a new araB gene that functions in yeasts, other than the known araB derived from Lactobacillus plantarum.

(2) Screening of araD and araA genes Thereafter, in order to search for new araA and araD sequences, araA and araD genes derived from Bacillus licheniformis, which are known as bacteria having the ability to assimilate arabinose, were focused. There are two types for each of araA and araD genes derived from Bacillus licheniformis in the genome.

Neither of the two types of araD genes derived from Bacillus licheniformis that function in yeasts and contribute to the assimilation of arabinose are known, and it was found that the araD1 gene had particularly excellent in arabinose assimilation characteristics. At this time, a plurality of other new araD genes were also found.

Then, new araA genes were searched for using the araD1 gene derived from Bacillus licheniformis. Although the araA1 derived from Bacillus licheniformis is known, it was found that the araA2 gene, which has never been reported in yeasts, can be tried and used. At this time, a plurality of new araA genes were additionally found.

1. Method

1.1. Test Strain

A strain with araA, araB, and araD genes introduced was produced using a wine yeast S. cerevisiae OC-2 strain with enhanced expression of an arabinose transporter gene (GAL2) as the parent strain. Table 1 shows the araA, araB, and araD genes used in this example.

TABLE 1

| Gene | | | Nucleotide | Amino acid | |
|---|---|---|---|---|---|
| name | Origin | Accession No. | sequence | sequence | Source |
| LParaB | Lactobacillus plantarum | WP_011102218 | SEQ ID NO: 27 | SEQ ID NO: 28 | Literature [1] |
| LCaraB | Lactobacillus composti | WP_035452766 | SEQ ID NO: 29 | SEQ ID NO: 30 | |
| LFaraB | Lactobacillus | WP-033614555 | SEQ ID NO: 31 | SEQ ID NO: 32 | |
| | fabifermentans | | | | |
| LSaraB | Lactobacillus sakei | WP_016265794 | SEQ ID NO: 33 | SEQ ID NO: 34 | |
| PLaraB | Pediococcus lolii | GAC46306 | SEQ ID NO: 35 | SEQ ID NO: 36 | |
| TSaraB | Thermoactinomyces sp. | WP_049720024 | SEQ ID NO: 7 | SEQ ID NO: 8 | |
| BCaraB | Bacillus coagulans | AJO22070 | SEQ ID NO: 37 | SEQ ID NO: 38 | |
| MCaraB | Megasphaera cerevisiae | WP_048515518 | SEQ ID NO: 15 | SEQ ID NO: 16 | |
| CNaraB | Clostridium nexile | CDC22812 | SEQ ID NO: 9 | SEQ ID NO: 10 | |
| SSaraB | Selenomonas sp. | WP_050342034 | SEQ ID NO: 11 | SEQ ID NO: 12 | |
| | Oral taxon | | | | |
| PSaraB | Paenibacillus sp. | WP_039877980 | SEQ ID NO: 13 | SEQ ID NO: 14 | |
| LParaD | Lactobacillus plantarum | WP_003642916 | SEQ ID NO: 39 | SEQ ID NO: 40 | Literature [1] |
| BLaraD1 | Bacillus licheniformis | WP_003182291 | SEQ ID NO: 17 | SEQ ID NO: 18 | |
| BLaraD2 | Bacillus licheniformis | WP_011198185 | SEQ ID NO: 41 | SEQ ID NO: 42 | |
| AHaraD | Anaerostipes hadrus | WP_009204419 | SEQ ID NO: 43 | SEQ ID NO: 44 | |
| AParaD | Alkalibacterium | WP_091486828 | SEQ ID NO: 19 | SEQ ID NO: 20 | |
| | putridalgicola | | | | |
| BAaraD | Bacillus acidiproducens | WP_018662662 | SEQ ID NO: 45 | SEQ ID NO: 46 | |
| CS17araD | Carnobacterium sp. 17-4 | WP_013709965 | SEQ ID NO: 21 | SEQ ID NO: 22 | |
| FParaD | Fructobacillus | WP_059376677 | SEQ ID NO: 47 | SEQ ID NO: 48 | |
| | pseudoficulneus | | | | |
| LlaraD | Listeria ivanovii | WP_038406726 | SEQ ID NO: 49 | SEQ ID NO: 50 | |
| MSaraD | Megasphaera sp. An286 | WP_087476851 | SEQ ID NO: 51 | SEQ ID NO: 52 | |
| SSaraD | Selenomonas sp. | WP_009442966 | SEQ ID NO: 53 | SEQ ID NO: 54 | |
| | oral taxon 149 | | | | |
| BLaraA1 | Bacillus licheniformis | WP_003184257 | SEQ ID NO: 55 | SEQ ID NO: 56 | Literature [2] |
| BLaraA2 | Bacillus licheniformis | WP_011198012 | SEQ ID NO: 1 | SEQ ID NO: 2 | |
| SRaraA | Selenomonas | WP_072306024 | SEQ ID NO: 3 | SEQ ID NO: 4 | |
| | ruminantium | | | | |
| LLaraA | Lactococcus lactis | SBW30785 | SEQ ID NO: 57 | SEQ ID NO: 58 | |
| MCaraA | Megasphaera cerevisiae | WP_048515519 | SEQ ID NO: 59 | SEQ ID NO: 60 | |
| CAaraA1 | Clostridium sp. | WP_010964651 | SEQ ID NO: 61 | SEQ ID NO: 62 | Literature [2] |
| CAaraA2 | Clostridium | WP_034583464 | SEQ ID NO: 63 | SEQ ID NO: 64 | Literature [2] |
| | acetobutylicum | | | | |
| BAaraA | Bacillus akibai | WP_035662676 | SEQ ID NO: 65 | SEQ ID NO: 66 | |
| BHaraA | Bacillus halodurans | WP_010898034 | SEQ ID NO: 67 | SEQ ID NO: 68 | |
| LSaraA | Lactobacillus sakei | WP_011375537 | SEQ ID NO: 5 | SEQ ID NO: 6 | |
| OOaraA | Oenococcus oeni | WP_002822487 | SEQ ID NO: 69 | SEQ ID NO: 70 | |

Literature [1]: Wisselink, H. W et al. Appl. Environ. Microbiol. (2007) 73: 4881-4891
Literature [2]: U.S. Pat. No. 8,753,862 B2

Table 2 summarizes the names and genotypes of the strains produced in this example and subjected to the fermentation test for evaluation of the ability to assimilate arabinose.

TABLE 2

| Strain name | Genotype |
|---|---|
| Uz2837 | SUC2/SUC2::GAL2 |
| Uz2839 | SUC2/SUC2::GAL2 ATH1/ath1::LParaD PDC6/PDC6::LParaA |
| Uz2875 | SUC2/SUC2::GAL2 ATH1/ath1::LParaD PDC6/PDC6::LParaA GAD1/GAD1::LParaB |
| Uz2861 | SUC2/SUC2::GAL2 ATH1/ath1::LParaD PDC6/PDC6::LParaA GAD1/GAD1::CNaraB |
| Uz2863 | SUC2/SUC2::GAL2 ATH1/ath1::LParaD PDC6/PDC6::LParaA GAD1/GAD1::LCaraB |
| Uz2864 | SUC2/SUC2::GAL2 ATH1/ath1::LParaD PDC6/PDC6::LParaA GAD1/GAD1::LFaraB |
| Uz2865 | SUC2/SUC2::GAL2 ATH1/ath1::LParaD PDC6/PDC6::LParaA GAD1/GAD1::LSaraB |
| Uz2866 | SUC2/SUC2::GAL2 ATH1/ath1::LParaD PDC6/PDC6::LParaA GAD1/GAD1::PLaraB |
| Uz2867 | SUC2/SUC2::GAL2 ATH1/ath1::LParaD PDC6/PDC6::LParaA GAD1/GAD1::TSaraB |
| Uz2868 | SUC2/SUC2::GAL2 ATH1/ath1::LParaD PDC6/PDC6::LParaA GAD1/GAD1::BCaraB |
| Uz2869 | SUC2/SUC2::GAL2 ATH1/ath1::LParaD PDC6/PDC6::LParaA GAD1/GAD1::MCaraB |
| Uz2870 | SUC2/SUC2::GAL2 ATH1/ath1::LParaD PDC6/PDC6::LParaA GAD1/GAD1::SSaraB |
| Uz2871 | SVC2/SUC2::GAL2 ATH1/ath1::LParaD PDC6/PDC6::LParaA GAD1/GAD1::PSaraB |
| Uz2943 | SUC2/SUC2::GAL2 PDC6/PDC6::BLaraA2 GAD1/GAD1::SsaraB |
| Uz3003 | SUC2/SUC2::GAL2 PDC6/PDC6::BLaraA2 GAD1/GAD1::SsaraB ATH1/ath1::BLaraD1 |
| Uz3010 | SUC2/SUC2::GAL2 PDC6/PDC6::BLaraA2 GAD1/GAD1::SsaraB ATH1/ath1::BLaraD2 |
| Uz3011 | SUC2/SUC2::GAL2 PDC6/PDC6::BLaraA2 GAD1/GAD1::SsataB ATH1/ath1::LParaD |
| Uz3121 | SUC2/SUC2::GAL2 PDC6/PDC6::BLaraA2 GAD1/GAD1::SsaraB ATH1/ath1::AHaraD |
| Uz3122 | SUC2/SUC2::GAL2 PDC6/PDC6::BLaraA2 GAD1/GAD1::SsaraB ATH1/ath1::AParaD |
| Uz3123 | SUC2/SUC2::GAL2 PDC6/PDC6::BLaraA2 GAD1/GAD1::SsaraB ATH1/ath1::BAaraD |
| Uz3124 | SUC2/SUC2::GAL2 PDC6/PDC6::BLaraA2 GAD1/GAD1::SsaraB ATH1/ath1::CS17araD |
| Uz3126 | SUC2/SUC2::GAL2 PDC6/PDC6::BLaraA2 GAD1/GAD1::SsaraB ATH1/ath1::FParaD |
| Uz3127 | SUC2/SUC2::GAL2 PDC6/PDC6::BLaraA2 GAD1/GAD1::SsaraB ATH1/ath1::LlaraD |
| Uz3128 | SUC2/SUC2::GAL2 PDC6/PDC6::BLaraA2 GAD1/GAD1::SsaraB ATH1/ath1::MSaraD |
| Uz3129 | SUC2/SUC2::GAL2 PDC6/PDC6::BLaraA2 GAD1/GAD1::SsaraB ATH1/ath1::SSaraD |
| Uz3151 | SUC2/SUC2::GAL2 GAD1/GAD1::SsaraB ATH1/ath1::BLaraD1 |
| Uz3181 | SUC2/SUC2::GAL2 GAD1/GAD1::SsaraB ATH1/ath1::BLaraD1 PDC6/PDC6::LParaA |
| Uz3182 | SUC2/SUC2::GAL2 GAD1/GAD1::SsaraB ATH1/ath1::BLaraD1 PDC6/PDC6::BLaraA2 |
| Uz3183 | SUC2/SUC2::GAL2 GAD1/GAD1::SsaraB ATH1/ath1::BLaraD1 PDC6/PDC6::BLaraA1 |
| Uz3184 | SUC2/SUC2.:GAL2 GAD1/GAD1::SsaraB ATH1/ath1::BLaraD1 PDC6/PDC6::CAaraA1 |
| Uz3186 | SUC2/SUC2::GAL2 GAD1/GAD1::SsaraB ATH1/ath1::BLaraD1 PDC6/PDC6::CAaraA2 |
| Uz3188 | SUC2/SUC2::GAL2 GAD1/GAD1::SsaraB ATH1/ath1::8LaraD1 PDC6/PDC6::SRaraA |
| Uz3189 | SUC2/SUC2::GAL2 GAD1/GAD1::SsaraB ATH1/ath1::BLaraD1 PDC6/PDC6::BAaraA |
| Uz3190 | SUC2/SUC2::GAL2 GAD1/GAD1::SsaraB ATH1/ath1::BLaraD1 PDC6/PDC6::BHaraA |
| Uz3191 | SUC2/SUC2::GAL2 GAD1/GAD1::SsaraB ATH1/ath1::BLaraD1 PDC6/PDC6::LLaraA |
| Uz3192 | SUC2/SUC2::GAL2 GAD1/GAD1::SsaraBATH1/ath1::BLaraD1 PDC6/PDC6::LSaraA |
| Uz3193 | SUC2/SUC2::GAL2 GAD1/GAD1::SsaraBATH1/ath1::BLaraD1 PDC6/PDC6::MCaraA |
| Uz3194 | SUC2/SUC2::GAL2 GAD1/GAD1::SsaraBATH1/ath1::BLaraD1 PDC6/PDC6::OOaraA |
| Uz3096 | ALD6/ALD6-T_GIC1 adh2::ADH1_eutE GRE3/gre3::TKL1_TAL1_RPE1_RKI1_XI~N337C_XKS1 |
| Uz3337 | GAL1/GAL1::GAL2 ALD6/ALD6-T_GIC1 adh2::ADH1_eutE |
| | GRE3/gre3::TKL1_TAL1_RPE1_RKI1_XI~N337C_XKS1 |
| Uz3338 | GAL1/GAL1::GAL2 ALD6/ALD6-T_GIC1 adh2::ADH1_eutE |
| | GRE3/gre3::TKL1_TAL1_RPE1_RKI1_XI~N337C_XKS1 |

1.2. Production of Plasmid for GAL2 Gene Expression

A plasmid: pUC-5U500_SUC2-P_HOR7-GAL2-T_DIT1-loxP-HPH-loxP-5U_SUC2 having a sequence necessary for introducing a GAL2 gene derived from Saccharomyces cerevisiae was produced.

This plasmid was constructed so as to comprise, at 5′ side, a GAL2 gene in which HOR7 promoter and DIT1 terminator derived from the Saccharomyces cerevisiae BY4742 strain were added, a DNA sequence about 1000 bp upstream of a SUC2 gene (5U500_SUC2) and a DNA sequence about 500 bp upstream of a SUC2 gene (5U_SUC2) as regions for homologous recombination on the yeast genome and introduction of the GAL2 gene, and a gene sequence containing an HPH gene as a marker (HPH marker). The marker gene had a sequence capable of marker removal by introducing LoxP sequences on both sides.

Each DNA sequence can be amplified by PCR using the primer shown in Table 3. In order to bind DNA fragments, a DNA sequence was added to the primer so as to overlap an adjacent DNA sequence by about 15 bp in Table 3. A target DNA fragment was amplified using such a primer with the synthetic DNA of the Saccharomyces cerevisiae OC2 genome and the LoxP sequences as templates. Using an In-Fusion HD Cloning Kit (available from Takara Bio Inc.), DNA fragments obtained were sequentially bound and cloned into a plasmid pUC19, to produce a plasmid as the final target.

1.3. Production of Plasmid for araA Gene Introduction

A plasmid: 5U_PDC6-P_HOR7-[araA]-T_RPL41B-LoxP66-P_TEF1-SAT-T_LEU2-P_GAL1-Crei-T_CYC1-LoxP71-3U_PDC6 having a sequence necessary for introducing various araA genes shown in Table 1 into the PDC6 gene locus of the yeast was produced. In [araA] in this plasmid name, the gene name shown in Table 1 is input.

This plasmid was constructed so as to comprise, at 5′ side, an araA gene (with the full length of sequence totally synthesized so that codons were converted according to the codon use frequency in the yeast) in which HOR7 promoter and RPL41B terminator derived from the Saccharomyces cerevisiae BY4742 strain were added, a DNA sequence in the region about 500 bp downstream of the 3′ end of the PDC6 gene (3U_PDC6), and a gene sequence containing a SAT gene as a marker (SAT marker). The marker gene had a sequence capable of marker removal by introducing LoxP sequences on both sides.

Each DNA sequence can be amplified by PCR using the primer shown in Table 3. In order to bind DNA fragments, a DNA sequence was added to the primer so as to overlap an adjacent DNA sequence by about 15 bp in Table 3. A target DNA fragment was amplified using such a primer with the synthetic DNA of the Saccharomyces cerevisiae OC2 genome, the araA gene, and the LoxP sequences as templates. Using an In-Fusion HD Cloning Kit (available from Takara Bio Inc.), DNA fragments obtained were sequentially bound and cloned into a plasmid pUC19, to produce a plasmid as the final target.

1.4. Production of Plasmid for araD Gene Introduction

A plasmid: 5U_ATH1-P_TDH3-[araD]-T_DIT1-LoxP66-P_CYC1-G418-T_URA3-LoxP71-3U_ATH1 having a sequence necessary for introducing various araD genes shown in Table 1 into the ATH1 gene locus of the yeast was produced. In [araD] in this plasmid name, the gene name shown in Table 1 is input.

This plasmid was constructed so as to comprise, at 5′ side, an araD gene (with the full length of sequence totally synthesized so that codons were converted according to the codon use frequency in the yeast) in which TDH3 promoter and DIT1 terminator derived from the Saccharomyces cerevisiae BY4742 strain were added, a DNA sequence in the region about 500 bp downstream of the 3′ end of the ATH1 gene (3U_ATH1), and a gene sequence containing a G418 gene as a marker (G418 marker). The marker gene had a sequence capable of marker removal by introducing LoxP sequences on both sides.

Each DNA sequence can be amplified by PCR using the primer shown in Table 3. In order to bind DNA fragments, a DNA sequence was added to the primer so as to overlap an adjacent DNA sequence by about 15 bp in Table 3. The target DNA fragment was amplified using such a primer with the synthetic DNA of the Saccharomyces cerevisiae OC2 genome, the araD gene, and the LoxP sequences as templates. Using an in-Fusion HD Cloning Kit (available from Takara Bio Inc.), DNA fragments obtained were sequentially bound and cloned into a plasmid pUC19, to produce a plasmid as the final target.

1.5. Plasmid for araB Gene Introduction

A plasmid: 5U500_GAD1-P_TDH3-[araB]-T_DIT1-LoxP-T_CYC1-Crei-P_SED1-T_LEU2-BSD-P_TEF1-LoxP-5U_GAD1 having a sequence necessary for introducing various araB genes shown in Table 1 into the GAD1 gene locus of the yeast was produced. In [araB] in this plasmid name, the gene name shown in Table 1 is input.

This plasmid was constructed so as to comprise, at 5′ side, an araB gene (with the full length of sequence totally synthesized so that codons were converted according to the codon use frequency in the yeast) in which TDH3 promoter and DIT1 terminator derived from the Saccharomyces cerevisiae BY4742 strain were added, a DNA sequence in the region about 1000 bp to 500 bp upstream of the 5′ end of the GAD1 gene (5U500_GAD1), a DNA sequence in the region about 500 bp upstream of the 5′ end of the GAD1 gene (5U_GAD1), and a gene sequence containing a BSD gene as a marker (BSD marker). The marker gene had a sequence capable of marker removal by introducing LoxP sequences on both sides. A Cre gene necessary for marker removal (NCBI access No. NP_415757.1, with the full length of sequence totally synthesized so that codons were converted according to the codon use frequency in the yeast) was introduced and fused with a promoter induced by galactose, GAL1 promoter.

Each DNA sequence can be amplified by PCR using the primer shown in Table 3. In order to bind DNA fragments, a DNA sequence was added to the primer so as to overlap an adjacent DNA sequence by about 15 bp in Table 3. The target DNA fragment was amplified using such a primer with the synthetic DNA of the Saccharomyces cerevisiae BY4742 genome, the araB gene, and the LoxP sequences as templates. Alternatively, a DNA fragment was synthesized so as to contain a sequence overlapping adjacent DNA sequence by about 15 bp (gblocks, available from Integrated DNA Technologies, Inc). Using an In-Fusion HD Cloning Kit (available from Takara Bio Inc.), DNA fragments obtained were sequentially bound and cloned into a plasmid pUC19, to produce a plasmid as the final target.

1.6. Plasmid for araA, araB, and araD Genes Introduction

A plasmid: 5U_GAL1-P_HOR7-BlaraA2 (or SRaraA)-T_RPL41B-T_DIT1-BlaraD1-P_TDH3-LoxP66-P_TEF1-SAT-T_LEU2-P_GAL1-Crei-T_CYC1-LoxP71-P_FBA1-SsaraB-T_RPL3-3U_GAL1 having a sequence necessary for introducing the araA, araB, and araD genes into the GAL1 gene locus of the yeast was produced.

This plasmid was constructed so as to comprise, at 5′ side, each araA gene (with the full length of sequence totally synthesized so that codons were converted according to the codon use frequency in the yeast) in which HOR7 promoter and RPL41B terminator derived from the Saccharomyces cerevisiae BY4742 strain were added, a DNA sequence in the region about 500 bp upstream of the 5′ end of the GAL1 gene (5U_GAL1), a DNA sequence in the region about 500 bp downstream of the 3′ end of the GAL1 gene (3U_GAL1), and a gene sequence containing a SAT1 gene as a marker (SAT marker). The marker gene had a sequence capable of marker removal by introducing LoxP sequences on both sides. A Cre gene necessary for marker removal (NCBI access No. NP_415757.1, with the full length of sequence totally synthesized so that codons were converted according to the codon use frequency in the yeast) was introduced and fused with a promoter induced by galactose, GAL1 promoter.

Each DNA sequence can be amplified by PCR using the primer shown in Table 3. In order to bind DNA fragments, a DNA sequence was added to the primer so as to overlap an adjacent DNA sequence by about 15 bp in Table 3. The target DNA fragment was amplified using such a primer with the synthetic DNA of the Saccharomyces cerevisiae BY4742 genome, each of the araA, araB, and araD genes, the LoxP sequences as templates. Alternatively, a DNA fragment was synthesized so as to contain a sequence overlapping an adjacent DNA sequence by about 15 bp (gblocks, available from Integrated DNA Technologies, Inc). Using an In-Fusion HD Cloning Kit (available from Takara Bio Inc.), DNA fragments obtained were sequentially bound and cloned into a plasmid pUC19, to produce a plasmid as the final target.

1.7. Production of Plasmid for GAL2 Gene Introduction

A plasmid: 5U_GAL1-P_HOR7-GAL2-T_DIT1-LoxP66-P_CYC1-HPH-T_URA3-LoxP71-3U_GAL1 having a sequence necessary for introducing a GAL2 gene into the GAL1 gene locus of the yeast was produced. This plasmid was constructed so as to comprise, at 5′ side, a GAL2 gene in which HOR7 promoter and DIT1 terminator derived from the Saccharomyces cerevisiae BY4742 strain were added, a DNA sequence in the region about 500 bp upstream of the 5′ end of the GAL1 gene (5U_GAL1), a DNA sequence in the region about 500 bp downstream of the 3′ end of the GAL1 gene (3U_GAL1), and a gene sequence containing an HPH gene as a marker (HPH marker). The marker gene had a sequence capable of marker removal by introducing LoxP sequences on both sides. A Cre gene necessary for marker removal (NCBI access No. NP_415757.1, with the full length of sequence totally synthesized so that codons were converted according to the codon use frequency in the yeast) was introduced and fused with a promoter induced by galactose, GAL1 promoter.

Each DNA sequence can be amplified by PCR using the primer shown in Table 3. In order to bind DNA fragments, a DNA sequence was added to the primer so as to overlap an adjacent DNA sequence by about 15 bp in Table 3. The target DNA fragment was amplified using such a primer with the synthetic DNA of the Saccharomyces cerevisiae BY4742 genome DNA and the LoxP sequences as templates. Using an In-Fusion HD Cloning Kit (available from Takara Bio Inc.) or the like, DNA fragments obtained were sequentially bound and cloned into a plasmid pUC19, to produce a plasmid as the final target.

1.8. Production of GAL2 Gene Expression Strain

Using the S. cerevisiae OC2 strain of a diploid yeast (NBRC2260) as a host and a fragment obtained by amplifying the homologous recombination sites of a plasmid pBluntEndTOPO-5U500_SUC2-P_HOR7-GAL2-T_DIT1-loxP-HPH-loxP-5U_SUC2 by PCR, transformation was performed. The yeast was transformed using Frozen-EZ Yeast Transformation II (ZYMO RESEARCH) according to the attached protocol.

The transformant obtained was applied to a YPD agar medium containing hygromycin, followed by purification of grown colonies. This was used as Uz2837 strain. It was confirmed that the transgene of each of the selected strains was heterogeneously (1 copy) recombined.

1.9. Production of a Strain Expressing araA and araD Genes and Disrupting ATH1 Gene Heterozygously

Using the Uz2837 strain as a host, a fragment obtained by amplifying the homologous recombination sites of a plasmid pUC-5U_PDC6-P_HOR7-LParaA-RPL41B-LoxP66-P_TEF1-SAT-T_LEU2-P_GAL1-Crei-T_CYC1-LoxP71-3U_PDC6 by PCR, and a fragment obtained by amplifying the homologous recombination sites of 5U_ATH1-P_TDH3-LParaD-T_DIT1-LoxP66-P_CYC1-G418-T_URA3-LoxP71-3U_ATH1 by PCR, transformation was performed. The transformant obtained was applied to a YPD agar medium containing nourseothricin and G418, followed by purification of grown colonies. This was used as Uz2839 strain. It was confirmed that the transgene of each of the selected strains was heterogeneously (I copy) recombined, and the ATH1 gene was heterozygously disrupted.

1.10. Production of Strains Expressing GAL2, araA, araB, and araD Genes

Using the Uz2839 strain as a host and a fragment obtained by amplifying a portion between the homologous recombination sites of each plasmid pUC-5U500_GAD1-P_TDH3-[araB]-T_DIT1-LoxP71-T_CYC1-Crei-P_SED1-T_LEU2-Bla-P_TEF1-LoxP66-5U_GAD1 ([araB]=each gene name, see Table 1) by PCR, transformation was performed. The transformant obtained was applied to a YPD agar medium containing blasticidin, followed by purification of grown colonies. The purified strains were respectively named Uz2861 to 2871 and 2875 strains (Table 2). It was confirmed that each strain was heterogeneously (1 copy) recombined.

1.11. Production of a Strain Expressing GAL2, araA, and araB genes and disrupting ATH1 gene heterozygously

Using the Uz2837 strain as a host, a fragment obtained by amplifying the homologous recombination site of a plasmid pUC-5U_PDC6-P_HOR7-BLaraA2-RPL41B-LoxP66-P_TEF1-SAT-T_LEU2-P_GAL1-Crei-T_CYC1-LoxP71-3U_PDC6 by PCR, and a fragment obtained by amplifying the homologous recombination site of pUC-5U500_GAD1-P_TDH3-SSaraB-T_DIT1-LoxP71-T_CYC1-Crei-P_SED1-T_LEU2-Bla-P_TEF1-LoxP66-5U_GAD1 by PCR, transformation was performed. The transformant obtained was applied to a YPD agar medium containing nourseothricin and blasticidin, followed by purification of grown colonies. This was used as Uz2943 strain. It was confirmed that the transgene of each of the selected strains was heterogeneously (1 copy) recombined.

1.12. Production of Strains Expressing GAL2, araA, araB, and araD Genes

Using a fragment obtained by amplifying a portion between the homologous recombination sites of each plasmid pUC19-5U_ATH1-P_TDH3-[araD]-T_DIT1-LoxP66-P_CYC1-G418-T_URA3-LoxP71-3U_ATH1 ([araD]=each gene name, see Table 1) by PCR, the Uz2943 strain was transformed. The transformant obtained was applied to a YPD agar medium containing blasticidin, followed by purification of grown colonies. The purified strains were respectively named Uz3003, 3010, 3011, and 3121 to 3129 strains (Table 2). It was confirmed that each strain was heterogeneously (1 copy) recombined.

1.13. Production of a Strain Expressing GAL2, araB, and araD Genes and Disrupting ATH1 Gene Heterozygously

Using the Uz2837 strain as a host, a fragment obtained by amplifying the homologous recombination site of a plasmid pUC19-5U_ATH1-P_TDH3-BLaraD1-T_DIT1-LoxP66-P_CYC1-G418-T_URA3-LoxP71-3U_ATH1 by PCR, and a fragment obtained by amplifying the homologous recombination site of pUC-5U500_GAD1-P_TDH3-SSaraB-T_DIT1-LoxP71-T_CYC1-Crei-P_SED1-T_LEU2-Bla-P_TEF1-LoxP66-5U_GAD1 by PCR, transformation was performed. The transformant obtained was applied to a YPD agar medium containing G418 and blasticidin, followed by purification of grown colonies. This was used as Uz3151 strain. It was confirmed that the transgene of each of the selected strains was heterogeneously (I copy) recombined.

1.14. Production of Strains Expressing GAL2, araA, araB, and araD Genes

Using a fragment obtained by amplifying a portion between the homologous recombination sites of each plasmid pUC-5U_PDC6-P_HOR7-[araA]-RPL41B-LoxP66-P_TEF1-SAT-T_LEU2-P_GAL1-Crei-T_CYC1-LoxP71-3U_PDC6 ([araA]=each gene name, see Table 1) by PCR, the Uz3151 strain was transformed. The transformant obtained was applied to a YPD agar medium containing nourseothricin, followed by purification of grown colonies. The purified strains were respectively named Uz3181 to 3194 strains (Table 2). It was confirmed that each strain was heterogeneously (1 copy) recombined.

1.15. Production of a Strain in which Arabinose Assimilation Genes (GAL2, araA, araB, and araD Genes) were Introduced into Strain Having Ability to Assimilate Xylose and GAL1 Genes were Homozygously Disrupted

Using a plasmid 5U_GAL1-P_HOR7-GAL2-T_DIT1-LoxP66-P_CYC1-HPH-T_URA3-LoxP71-3U_GAL1, a Uz3096 strain having the ability to assimilate xylose was transformed. The transformant obtained was applied to a YPD agar medium containing hygromycin, followed by purification of grown colonies. The purified strain was named Uz3337 strain. It was confirmed that the transgene of each of the selected strains was heterogeneously (I copy) recombined, and the GAL1 gene was heterogeneously disrupted.

Then, using a plasmid pUC19-5U_GAL1-P_HOR7-BlaraA2-T_RPL41B-T_DIT1-BlaraD1-P_TDH3-LoxP66-P_TEF1-SAT-T_LEU2-P_GAL1-Crei-T_CYC1-LoxP71-P_FBA1-SsaraB-T_RPL3-3U_GAL1, the Uz3337 strain was transformed. The transformant obtained was applied to a YPD agar medium containing nourseothricin, followed by purification of grown colonies. The purified strain was named Uz3380 strain. Likewise, using a plasmid pUC19-5U_GAL1-P_HOR7-SRaraA-T_RPL41B-T_DIT1-BlaraD1-P_TDH3-LoxP66-P_TEF1-SAT-T_LEU2-P_GAL1-Crei-T_CYC1-LoxP71-P_FBA1-SsaraB-T_RPL3-3U_GAL1, the Uz3337 strain was transformed. The transformant obtained was applied to a YPD agar medium containing nourseothricin, followed by purification of grown colonies. The purified strain was named Uz3381 strain. It was confirmed that the transgene of each of the selected strains was heterogeneously (1 copy) recombined, and the GAL1 gene was homozygously disrupted.

TABLE 3

Amplified DNA

| | |
|---|---|
| fragment | Primer sequence (5′-3′) |

pUC-5U500_SUC2-P_HOR7-GAL2-T_DIT1_loxP-HPH-loxP-5U_SUC2

| | | |
|---|---|---|
| 5U500_SUC2 | CTGGAATTCGCCCTTTTGAGGTTATAGGGGCTT | SEQ ID NO: |
| | AGCATC | 71 |
| | GACCCGTGGCTGCGAGTCAGTTTTTCCAGAAA | SEQ ID NO: |
| | CCTCCATG | 72 |
| HOR7 promoter | TCGCAGCCACGGGTCAAC | SEQ ID NO: |
| | | 73 |
| | TTTTATTATTAGTCTTTTTTTTTTTTGACAATATC | SEQ ID NO: |
| | TG | 74 |
| GAL2 | ATGGCAGTTGAGGAGAACAATATG | SEQ ID NO: |
| | | 75 |
| | TTATTCTAGCATGGCCTTGTACCA | SEQ ID NO: |
| | | 76 |
| DIT1 terminator | GCCATGCTAGAATAATAAAGTAAGAGCGCTACA | SEQ ID NO: |
| | TTGGTCTACC | 77 |
| | TTACTCCGCAACGCTTTTCTGAAC | SEQ ID NO: |
| | | 78 |
| LoxP | TGACGGTATCGATAAGCTTGATATC | SEQ ID NO: |
| (including linker | | 79 |
| sequence) | ATAACTTCGTATAGCATACATTATACGAAGTTAT | SEQ ID NO: |
| | ACGACATCGTCGAATATGATTCAGGGTAAC | 80 |
| CYC1 promoter | ACGACATCGTCGAATATGATTC | SEQ ID NO: |
| | | 81 |
| | TATTAATTTAGTGTGTGTATTTGTGTTTGTGTG | SEQ ID NO: |
| | | 82 |
| HPH | CACACTAAATTAATAATGAAAAAGCCTGAACTC | SEQ ID NO: |
| | ACC | 83 |
| | TTTAGTAGACATGCACTATTCCTTTGCCCTCGG | SEQ ID NO: |
| | | 84 |
| URA3 terminator | TGCATGTCTACTAAACTCACAAATTAGAGCTTC | SEQ ID NO: |
| | AATT | 85 |
| | GGGTAATAACTGATATAATTAAATTG | SEQ ID NO: |
| | | 86 |
| LoxP | ATTATACGAAGTTATTGACACCGATTATTTAAAG | SEQ ID NO: |
| (including linker | CTG | 87 |
| sequence) | ATAATGTATGCTATACGAAGTTATGGGTAATAA | SEQ ID NO: |
| | CTGATATAATTAAATTGAAGC | 88 |
| 5U_SUC2 | AAAGCTGCAGCATACGAGGAATGTGATTATAAA | SEQ ID NO: |
| | TCCCTTTATG | 89 |
| | GCAGAATTCGCCCTTCATATACGTTAGTGAAAA | SEQ ID NO: |
| | GAAAAGCTTTTTG | 90 |
| pUC19 | AAGGGCGAATTCTGCAGATATCC | SEQ ID NO: |
| | | 91 |
| | AAGGGCGAATTCCAGCACACTG | SEQ ID NO: |
| | | 92 |

pUC-5U500_GAD1-P_TDH3-XaraB-T_DIT1-LoxP-T_CYC1-Cre-P_GAL1-T_LEU2-BSD-
P_TEF1-LoxP-5U_GAD1

| | | |
|---|---|---|
| 5U500_GAD1 | ACGGCCAGTGAATTCGGCTGATGTAATGGTATT | SEQ ID NO: |
| | GTTATTCPACC | 93 |
| | ATTTACCAGCATCAGCGCC | SEQ ID NO: |
| | | 94 |
| TDH3 promotor | CTGATGCTGGTAAATTAGCGTTGAATGTTAGCG | SEQ ID NO: |
| | TCAAC | 95 |
| | TTTGTTTGTTTATGTGTGTT | SEQ ID NO: |
| | | 96 |
| LParaB | ACATAAACAAACAAAATGAATTTGGTCGAAACC | SEQ ID NO: |
| | GC | 97 |
| | AGCGCTCTTACTTTATTAGTATTTAATAGCTTGA | SEQ ID NO: |
| | CCAGCGGC | 98 |
| LCaraB | ATGAACACTCTGGAGATATCAAAGGCG | SEQ ID NO: |
| | | 99 |
| | TCACCCTTTCTCGTTTATAGCATCCC | SEQ ID NO: |
| | | 100 |
| LFaraB | ATGAACTTGATCGAGACGAGTCAGG | SEQ ID NO: |
| | | 101 |
| | TCAGGATTTAACCGTTTCGCCCGC | SEQ ID NO: |
| | | 102 |
| LSaraB | ATGAACTTGGTTGAGATTGCACAAGC | SEQ ID NO: |
| | | 103 |
| | TCACTTCTCATCTTTTATCGCGGCG | SEQ ID NO: |
| | | 104 |
| PLaraB | ATGGAAATCTTGAAAATGAACATCG | SEQ ID NO: |
| | | 105 |
| | TTATATCACTTCACCAGCACGAGC | SEQ ID NO: |
| | | 106 |
| MCaraB | ATGGGTTTGATGAATGTCGCTGC | SEQ ID NO: |
| | | 107 |
| | CTACTCTGCTAATGCCGTACCAGC | SEQ ID NO: |
| | | 108 |
| SSaraB | ATGGACATGACCGCCGC | SEQ ID NO: |
| | | 109 |
| | AGCGCTCTTACTTTACTAGCCGTTGTAAGTCAA | SEQ ID NO: |
| | CGCG | 110 |
| PSaraB | ATGGATCAGAACATCCGTCAAGCG | SEQ ID NO: |
| | | 111 |
| | CTACCCCCTCCCGTTTTCTATTAAATG | SEQ ID NO: |
| | | 112 |
| CNaraB | ATGGGTAACGTAAAGGAAACG | SEQ ID NO: |
| | | 113 |
| | TTAAACCAGGTTCTTCAAAGCGC | SEQ ID NO: |
| | | 114 |
| DIT1 terminator | TAAAGTAAGAGCGCTACATTGGTCTACC | SEQ ID NO: |
| | | 115 |
| | TTACTCCGCAACGCTTTTCTGAAC | SEQ ID NO: |
| | | 116 |
| LoxP | TATAATGTATGCTATACGAAGTTATAGCTTGCA | SEQ ID NO: |
| (including linker | AATTAAAGCCTTCGAGCGTCCCAAAACCTTC | 117 |
| sequence) | ATAGCATACATTATACGAACGGTATGACACCGA | SEC) ID NO: |
| | TTATTTAAAGCTGCAG | 118 |
| CYC1 terminator | AGCTTGCAAATTAAAGCCTTCG | SEQ ID NO: |
| | | 119 |
| | TTAGTTATGTCACGCTTACATTCACG | SEQ ID NO: |
| | | 120 |
| Cre | GCGTGACATAACTAATCAATCACCATCTTCCAA | SEQ ID NO: |
| | CAATC | 121 |
| | CAAGGAGAAAAAACCATGTCTAACTTGTTGACT | SEQ ID NO: |
| | GTTC | 122 |
| GALA promoter | GGTTTTTTCTCCTTGACGTTAAAGTATAG | SEQ ID NO: |
| | | 123 |
| | TGCATGTCTACTAAACTCACAAATTAGAGCTTC | SEQ ID NO: |
| | AATTTAATTATATCAGTTATTACCCACGGATTAG | 124 |
| | AAGCCGCCG | |
| URA3 terminator | GGGTAATAACTGATATAATTAAATTG | SEQ ID NO: |
| | | 125 |
| | TGCATGTCTACTAAACTCACAAATTAGAG | SEQ ID NO: |
| | | 126 |
| BSD | TTAGCCCTCCCACACATAACCAG | SEQ ID NO: |
| | | 127 |
| | CATGGCCAAGCCTTTGTCTCAAG | SEQ ID NO: |
| | | 128 |
| TEF1 prometor | CCCTTAGATTAGATTGCTATGCTTTCTTTCTAAT | SEQ ID NO: |
| | G | 199 |
| | ATAGCATACATTATACGAAGTTATCCCACACAC | SEQ ID NO: |
| | CATAGCTTCAAAATG | 130 |
| LoxP | TACGAACGGTAAGGGAAAGATATGAG | SEQ ID NO: |
| (including linker | | 131 |
| sequence) | ATAGCATACATTATACGAAGTTATCCCACACAC | SEQ ID NO: |
| | CATAGCTTCAAAATG | 132 |
| 5U_GAD1 | TCTAGTTGGTTCTTGACATTTTTCAAATAATC | SEQ ID NO: |
| | | 133 |
| | TCCCCGGGTACCGAGTATTCCTTGTTTTGTTCA | SEQ ID NO: |
| | GCCTG | 134 |
| pUC19 | ACCCGGGGATCCTCTAGAGTCG | SEQ ID NO: |
| | | 135 |
| | GAATTCACTGGCCGTCGTTTTAC | SEQ ID NO: |
| | | 136 |

pUC19-5U_ATH1-P_TDE13-XaraD-T_DIT1-LoxP66-P_CYCl-G418-T_URA3-LoxP71-
3U_ATH1

| | | |
|---|---|---|
| 5U_ATH1 | CTGGAATTCGCCCTTGTATGACCACATTCTATA | SEQ ID NO: |
| | CTGAGAAGAGTGCC | 137 |
| | TAACATTCAACGCTATATTGGAATGAGGAAATT | SEQ ID NO: |
| | TCGGTAAAAAC | 138 |
| TDH3 promotor | TAGCGTTGAATGTTAGCGTCAACAAC | SEQ ID NO: |
| | | 139 |
| | TTTGTTTGTTTATGTGTGTTTATTCGAAAC | SEQ ID NO: |
| | | 140 |
| LParaD | ACATAAACAAACAAAATGTTGGAAGCATTGAAG | SEQ ID NO: |
| | CAAG | 141 |
| | AGCGCTCTTACTTTATTACTTTCTAACAGCGTG | SEQ ID NO: |
| | ATCTTTTGAATG | 142 |
| BLaraD1 | ACATAAACAAACAAAATGTTGGAGCAGTTAAAG | SEQ ID NO: |
| | GAAGAAG | 143 |
| | AGCGCTCTTACTTTATTATTTCTGACCGTAATAG | SEQ ID NO: |
| | GCATTCTTAC | 144 |
| BLaraD2 | ACATAAACAAACAAAATGTTGGAAAGCCTAAAG | SEQ ID NO: |
| | GAACAAG | 145 |
| | AGCGCTCTTACTTTATTATTGGCCATAGTATGC | SEQ ID NO: |
| | ATCAGCTC | 146 |
| AHaraD | ATGCTTGAACAGTTGAAGAAAGAAG | SEQ ID NO: |
| | | 147 |
| | TCATTTACCTTGACCATAATAGGC | SEQ ID NO: |
| | | 148 |
| AParaD | ATGCTAGAAAAGTTAAAGCAGG | SEQ ID NO: |
| | | 149 |
| | TCATTGACCGTAGTAAGCATTCTC | SEQ ID NO: |
| | | 150 |
| BAaraD | ATGTTGGAAGAATTAAAGAAAG | SEQ ID NO: |
| | | 151 |
| | TCACTTCGTOTGACCGTAGTAAGC | SEQ ID NO: |
| | | 152 |
| CS17araD | ATGCTGGAACAACTTAAGGAGGAAG | SEQ ID NO: |
| | | 153 |
| | TCAGTGCTTATTTTTTTGACCGTAG | SEQ ID NO: |
| | | 154 |
| FParaD | ATGTTGTTGGAAAAGCTGAGGCTGG | SEQ ID NO: |
| | | 155 |
| | TCAAGCTTGCCCGTAGTAGGC | SEQ ID NO: |
| | | 156 |
| LlaraD | ATGTTAGAAGCCCTAAAGGAAG | SEQ ID NO: |
| | | 157 |
| | TCACTTTTGACCGTAGTAAGCATC | SEQ ID NO: |
| | | 158 |
| MSaraD | ATGTTGGAGGAACTAAAGCAGCAGG | SEQ ID NO: |
| | | 159 |
| | TCATTTTTTCTGACCGTAGTAAGC | SEQ ID NO: |
| | | 160 |
| SSaraD | ATGCTAGAAGAGTTAAAGCAAGAGG | SEQ ID NO: |
| | | 161 |
| | TCAGGCTTTTTGGCCATAGTAAGC | SEQ ID NO: |
| | | 162 |
| DIT1 termnatcr | GCCATGCTAGAATAATAAAGTAAGAGCGCTACA | SEQ ID NO: |
| | TTGGTCTACC | 163 |
| | TTACTCCGCAACGCTTTTCTGAAC | SEQ ID NO: |
| | | 164 |
| LoxP | TGACGGTATCGATAAGCTTGATATC | SEQ ID NO: |
| (including linker | | 165 |
| sequence) | ATAACTTCGTATAGCATACATTATACGAAGTTAT | SEQ ID NO: |
| | ACGACATCGTCGAATATGATTCAGGGTAAC | 166 |
| CYC1 promoter | ACGACATCGTCGAATATGATTC | SEQ ID NO: |
| | | 167 |
| | TATTAATTTAGTGTGTGTATTTGTGTTTGTGTG | SEQ ID NO: |
| | | 168 |
| G418 | ATGAGCCATATTCAACGGGAAAC | SEQ ID NO: |
| | | 169 |
| | TTTAGTAGACATGCATTACAACCAATTAACCAAT | SEQ ID NO: |
| | TCTG | 170 |
| URA3 terminator | TGCATGTCTACTAAACTCACAAATTAGAGCTTC | SEQ ID NO: |
| | AATT | 171 |
| | GGGTAATAACTGATATAATTAAATTG | SEQ ID NO: |
| | | 172 |
| LoxP | ATTATACGAAGTTATTGACACCGATTATTTMAG | SEQ ID NO: |
| (including linker | CTG | 173 |
| sequence) | ATAATGTATGCTATACGAAGTTATGGGTAATAA | SEQ ID NO: |
| | CTGATATAATTAAATTGAAGC | 174 |
| 3U_ATH1 | AAAGCTGCAGCATACATGAAATGATGCATATAA | SEQ ID NO: |
| | GTAGCGC | 175 |
| | GCAGAATTCGCCCTTAGTGTTTGCTTAATTTAC | SEQ ID NO: |
| | ATAGGACCC | 176 |
| pUC19 | AAGGGCGAATTCTGCAGATATCC | SEQ ID NO: |
| | | 177 |
| | AAGGGCGAATTCCAGCACACTG | SEQ ID NO: |
| | | 178 |

pUC-5U_PDC6-P_HOR7-XaraA-T_RPL41B-LoxP66-P_TEF1-SAT-T_LEU2-P_GAL1-Crei-
T_CYC1-LexP71-3U_PDC6

| | | |
|---|---|---|
| 5U_PDC6 | CTACCCTATTTTCTCTTACCAGCGAAC | SEQ ID NO: |
| | | 179 |
| | GACCCGTGGCTGCGATTTTGGCCAAATGCCAC | SEQ ID NO: |
| | AG | 180 |
| HOR7 promotor | TCGCAGCCACGGGTCAAC | SEC) ID NO: |
| | | 181 |
| | TTTTATTATTAGTCTTTTTTTTTTTTGACAATATC | SEQ ID NO: |
| | TG | 182 |
| LParaA | AGACTAATAATAAAAATGTTGTCCGTTCCAGAT | SEQ ID NO: |
| | TATGAATTTTG | 183 |
| | TTGCTCTCAATCCGCTTATTTTAAGAAAGCCTTT | SEQ ID NO: |
| | GTCATACCAAC | 184 |
| BLaraA2 | AGACTAATAATAAAAATGTTGACCACTGGTAAG | SEQ ID NO: |
| | AAGGAG | 185 |
| | TTGCTCTCAATCCGCTTATTTGATCACGACGTA | SEQ ID NO: |
| | TTCAAGATC | 186 |
| BLaraA1 | AGACTAATAATAAAAATGATCCAAGCCAAAACC | SEQ ID NO: |
| | CATG | 187 |
| | TTGCTCTCAATCCGCTTAGAATTTTCTCAATCTG | SEQ ID NO: |
| | TAAGCCGC | 188 |
| CAaraA1 | AGACTAATAATAAAAATGCTAAAGAACAAGAAG | SEQ ID NO: |
| | CTAGAATTTTG | 189 |
| | TTGCTCTCAATCCGCTTACCTTGATGTCTCTTTT | SEQ ID NO: |
| | ATAATGTCTCTCAA | 190 |
| CAaraA2 | AGACTAATAATAAAAATGTTGGAAAACAAGAAG | SEQ ID NO: |
| | ATGGAG | 191 |
| | TTGCTCTCAATCCGCTTATTTTGTGGTCTTTTCT | SEQ ID NO: |
| | ATTATGTCTCTTAGTTTAG | 192 |
| SRaraA | AGACTAATAATAAAAATGTTGGAAGTCAAGAAT | SEQ ID NO: |
| | TACGAATTCTG | 193 |
| | TTGCTCTCAATCCGCTCAGCAGATTTCAACCAA | SEQ ID NO: |
| | GTTGAC | 194 |
| BAaraA | ATGTTAACAATAAAGAAGTATCAATTCTGG | SEQ ID NO: |
| | | 195 |
| | TCATCTATCGAATTTCCTAGCGATG | SEQ ID NO: |
| | | 196 |
| BHaraA | ATGTTGCAAACGAAACCGTACAC | SEQ ID NO: |
| | | 197 |
| | TCACTTGAATAACCTTCTGAAG | SEQ ID NO: |
| | | 198 |
| LLaraA | ATGTTGGAAAACACTCAAAAGG | SEQ ID NO: |
| | | 199 |
| | TCAACCAAGGTTGATGTACGTC | SEQ ID NO: |
| | | 200 |
| LSaraA | ATGCTTAATACGGAAAACTACG | SEQ ID NO: |
| | | 201 |
| | TCATTTGATATTCACGTACGTC | SEQ ID NO: |
| | | 202 |
| MCaraA | ATGTTGCAGGTAAAAGAATATG | SEQ ID NO: |
| | | 203 |
| | TCAACAAATTTCCACTAGTTCC | SEQ ID NO: |
| | | 204 |
| OOaraA | ATGCTTAAGACAAATGACTACAAG | SEQ ID NO: |
| | | 205 |
| | TCACTCGGAATCAACAGCGAAAGTC | SEQ ID NO: |
| | | 206 |
| RPL41B terminator | GCGGATTGAGAGCAAATCGTTAAGT | SEQ ID NO: |
| | | 207 |
| | CTATACAGCGGAATTAGAGGCATAGCGGCAAA | SEQ ID NO: |
| | CTAAG | 208 |
| LoxP | ATAGCATACATTATACGAAGTTATCCCACACAC | SEQ ID NO: |
| (including linker | CATAGCTTCAAAATG | 209 |
| sequence) | ATAATGTATGCTATACGAACGGTAAGGGAAAGA | SEQ ID NO: |
| | TATGAGCTATACAGCG | 210 |
| TEF1 promotor | CCCACACACCATAGCTTCAAAATG | SEQ ID NO: |
| | | 211 |
| | ATCACCGAAATCTTCATGTTTAGTTCCTCACCTT | SEQ ID NO: |
| | | 212 |
| SAT | CAAGGTGAGGAACTAAACATGAAGATTTCGGT | SEQ ID NO: |
| | GAT | 213 |
| | TTAGGCGTCATCCTGTGCTC | SEQ ID NO: |
| | | 214 |
| LEU2 terminator | CAGGATGACGCCTAAAPAGATTCTCTTTTTTTAT | SEQ ID NO: |
| | GATATTTGTAC | 215 |
| | AGGAATCATAGTTTCATGATTTTCTGTTAC | SEQ ID NO: |
| | | 216 |
| GAL1 promotor | GAAACTATGATTCCTACGGATTAGAAGCCGCC | SEQ ID NO: |
| | G | 217 |
| | GGTTTTTTCTCCTTGACGTTAAAGTATAG | SEQ ID NO: |
| | | 218 |
| Cre | CAAGGAGAAAAAACCATGTCTAACTTGTTGACT | SEQ ID NO: |
| | GTTC | 219 |
| | GCGTGACATAACTAATCAATCACCATCTTCCAA | SEQ ID NO: |
| | CAATC | 220 |
| CYC1 terminator | TTAGTTATGTCACGCTTACATTCACG | SEQ ID NO: |
| | | 221 |
| | AGCTTGCAAATTAAAGCCTTCG | SEQ ID NO: |
| | | 222 |
| LoxP | ATAGCATACATTATACGAACGGTATGACACCGA | SEQ ID NO: |
| (including linker | TTATTTAAAGCTGCAG | 223 |
| sequence) | TATAATGTATGCTATACGAAGTTATAGCTTGCA | SEQ ID NO: |
| | AATTAAAGCTTCGAGCGTCCCAAAACCTTC | 224 |
| 3U_PDC6 | TGTTATAGAGTTCACACCTTATTCACATACTTTT | SEQ ID NO: |
| | TC | 225 |
| | GTGAACTCTATAACAGTATGCTGCAGCTTTAAA | SEQ ID NO: |
| | TAATCGGTG | 226 |
| pUC19 | AAGGGCGAATTCTGCAGATATCC | SEQ ID NO: |
| | | 227 |
| | AAGGGCGAATTCCAGCACACTG | SEQ ID NO: |
| | | 228 |

pUC19-5U_GAL1-P_HOR7-GAL2-T_DIT1-LoxP66-P_CYC1-HPH-T_URA3-LoxP71-
3U_GAL1

| | | |
|---|---|---|
| 5U_GAL1 | TGGCTACAGAATCATAAGTTGAATTCGAC | SEQ ID NO: |
| | | 299 |
| | GACCCGTGGCTGCGAGTTTTTTCTCCTTGACGT | SEQ ID NO: |
| | TAAAGTATAGAGG | 230 |
| HOR7 promotor | TCGCAGCCACGGGTCAAC | SEQ ID NO: |
| | | 231 |
| | CTCCTCAACTGCCATTTTTTATTATTAGTCTTTT | SEQ ID NO: |
| | TTTTTTTTGACAATATCTG | 232 |
| GAL2 | ATGGCAGTTGAGGAGAACAATATG | SEQ ID NO: |
| | | 233 |
| | TTATTCTAGCATGGCCTTGTACCAC | SEQ ID NO: |
| | | 234 |
| DIT1 terminator | GCCATGCTAGAATAATAAAGTAAGAGCGCTACA | SEQ ID NO: |
| | TTGGTCTACC | 235 |
| | CTATACAGOGGAATTTTACTCCGOAACGCTTTT | SEQ ID NO: |
| | C | 236 |
| LoxP | ATTATACGAAGTTATACGACATCGTCGAATATG | SEQ ID NO: |
| (including linker | ATTCAG | 237 |
| sequence) | ATAATGTATGCTATACGAACGGTAAGGGAAAGA | SEQ ID NO: |
| | TATGAGCTATACAGCG | 238 |
| CYC1 promoter | ACGACATCGTCGAATATGATTCAG | SEQ ID NO: |
| | | 239 |
| | TATTAATTTAGTGTGTGTATTTGTGTTTGTGTG | SEQ ID NO: |
| | | 240 |
| HPH | CACACTAAATTAATAATGAAAAAGCCTGAACTC | SEQ ID NO: |
| | ACC | 241 |
| | TTTAGTAGACATGCACTATTCCTTTGCCCTCGG | SEQ ID NO: |
| | | 242 |
| URA3 terminator | TGCATGTCTACTAAACTCACAAATTAGAGCTTC | SEQ ID NO: |
| | AATT | 243 |
| | GGGTAATAACTGATATAATTAAATTG | SEQ ID NO: |
| | | 244 |
| LoxP | ATTATACGAAGTTATTGACACCGATTATTTAAAG | SEQ ID NO: |
| (including linker | CTG | 245 |
| sequence) | ATAATGTATGCTATACGAAGTTATGGGTAATAA | SEQ ID NO: |
| | CTGATATAATTAAATTGAAGC | 246 |
| 3U_GAL1 | CTACTCATAACTTTAGCATCACAAAATACGC | SEQ ID NO: |
| | | 247 |
| | GTGAAATTAAGAAAGGAGTTTTATACAGATGAT | SEQ ID NO: |
| | ACC | 248 |
| pUC19 | ACCCGGGGATCCTCTAGAGTCG | SEQ ID NO: |
| | | 249 |
| | GAATTCACTGGCCGTCGTTTTAC | SEQ ID NO: |
| | | 250 |

pUC19-5U_GAL.1-P_HOR7-BlaraA2-T_RPLA1B-T_DIT1-BlaraD1 -P_TDH3-LoxP66-
P_TEF1-SAT-T_LEU2-P_GAL1-Crei-T_CYC1-LoxP71-P_FBAl-SsaraB-T_RPL3-3U_GAL1

| | | |
|---|---|---|
| 5U_GAL1 | TGGCTACAGAATCATAAGTTGAATTCGAC | SEQ ID NO: |
| | | 251 |
| | GACCCGTGGCTGCGAGTTTTTTCTCCTTGACGT | SEQ ID NO: |
| | TAAAGTATAGAGG | 252 |
| HOR7 promotor | TCGCAGCCACGGGTCAAC | SEQ ID NO: |
| | | 253 |
| | CTCCTCAACTGCCATTTTTTATTATTAGTCTTTT | SEQ ID NO: |
| | TTTTTTTTGACAATATCTG | 254 |
| BLaraA2 | AGACTAATAATAAAAATGTTGACCACTGGTAAG | SEQ ID NO: |
| | AAGGAG | 255 |
| | TTGCTCTCAATCCGCTTATTTGATCACGACGTA | SEQ ID NO: |
| | TTCAAGATC | 256 |
| SRaraA | AATACATATTCAAAATGGACATGACCGCCGCG | SEQ ID NO: |
| | GAAATAGTCAGAAATG | 257 |
| | TTCTAACAAAACTTCTAGCCGTTGTAAGTCAAC | SEQ ID NO: |
| | GCG | 258 |
| RPL41B terminator | GCGGATTGAGAGCAAATCGTTAAGT | SEQ ID NO: |
| | | 259 |
| | CTATACAGCGGAATTAGAGGCATAGCGGCAAA | SEQ ID NO: |
| | CTAAG | 260 |
| DIT1 terminator | GCCATGCTAGAATAATAAAGTAAGAGCGCTACA | SEQ ID NO: |
| | TTGGTCTACC | 261 |
| | CTATACAGCGGAATTTTACTCCGCAACGCTTTT | SEQ ID NO: |
| | C | 262 |
| BLaraD1 | TTCTAACAAAACTTCTTATTTCTGACCGTAATAG | SEQ ID NO: |
| | GCATTCTTAC | 263 |
| | TAATACATATTCAAATGTTGGAGCAGTTAAAG | SEQ ID NO: |
| | GAAGAAGTC | 264 |
| TDH3 promotor | TAGCGTTGAATGTTAGCGTCAACAAC | SEQ ID NO: |
| | | 265 |
| | TTTGTTTGTTTATGTGTGTTTATTCGAAAC | SEQ ID NO: |
| | | 266 |
| LoxP | TATAATGTATGCTATACGAAGTTATAGCTTGCA | SEQ ID NO: |
| (including linker | AATTAAAGCCTTCGAGCGTOCCAAAACCTTC | 267 |
| sequence) | ATAGCATACATTATACGAACGGTATGACACCGA | SEQ ID NO: |
| | TTATTTAAAGCTGCAG | 268 |
| CYC1 terminator | TTAGTTATGTCACGCTTACATTCACG | SEQ ID NO: |
| | | 269 |
| | AGCTTGCAAATTAAAGCCTTCG | SEQ ID NO: |
| | | 270 |
| Crei | GCGTGACATAACTAATCAATCACCATCTTCCAA | SEQ ID NO: |
| | CAATC | 271 |
| | CAAGGAGAAAAAACCATGTCTAACTTGTTGACT | SEQ ID NO: |
| | GTTC | 272 |
| GAL1 promoter | GGTTTTTTCTCCTTGACGTTAAAGTATAG | SEQ ID NO: |
| | | 273 |
| | TGCATGTCTACTAAACTCACAAATTAGAGCTTC | SEQ ID NO: |
| | AATTTAATTATATCAGTTATTACCCACGGATTAG | 274 |
| | AAGCCGCCG | |
| TEF1 promoter | CCCACACACCATAGCTTCAAAATG | SEQ ID NO: |
| | | 275 |
| | GTTTAGTTCCTCACCTTGTCGTATTATACTATG | SEQ ID NO: |
| | | 276 |
| SAT | ATGAAGATTTCGGTGATCCCTG | SEQ ID NO: |
| | | 277 |
| | TTAGGCGTCATCCTGTGCTC | SEQ ID NO: |
| | | 278 |
| LEU2 terminator | AAAGATTCTCTTTTTTTATGATATTTGTACATAA | SEQ ID NO: |
| | ACTTTATAAATG | 279 |
| | GGAATCATAGTTTCATGATTTTCTGTTAC | SEQ ID NO: |
| | | 280 |
| LoxP | TACGAACGGTAAGGGAAAGATATGAG | SEQ ID NO: |
| (including linker | | 281 |
| sequence) | ATAGCATACATTATACGAAGTTATCCCACACAC | SEQ ID NO: |
| | CATAGCTTCAAAATG | 282 |
| 3U_GAL1 | CTACTCATAACTTTAGCATCACAAAATACGC | SEQ ID NO: |
| | | 283 |
| | GTGAAATTAAGAAAGGAGTTTTATACAGATGAT | SEQ ID NO: |
| | ACC | 284 |
| pUC19 | ACCCGGGGATCCTCTAGAGTCG | SEQ ID NO: |
| | | 285 |
| | GAATTCACTGGCCGTCGTTTTAC | SEQ ID NO: |
| | | 286 |

1.16. Flask Fermentation Test

A test strain was inoculated in a 100-ml baffled flask in which 20 ml of a YPD liquid medium with a glucose concentration of 20 g/L (yeast extract: 10 g/L, peptone: 20 g/L, and glucose: 20 g/L) was dispensed, followed by culture at 30° C. and 120 rpm for 24 hours. After collecting the cells, the strain was inoculated on a 24-hole deep well plate in which 4.9 ml of a medium for producing ethanol was dispensed (bacteria concentration: 0.3 g of dried cells/L), followed by a fermentation test by shaking culture (230 rpm, amplitude 25 mm) at a temperature of 31° C. Each processing area of the 24-hole deep well plate was covered by a silicon lid with a check valve, so that a carbon dioxide gas generated escapes to the outside air, but oxygen does not enter from the outside, thereby maintaining each processing area to be anaerobic.

The concentrations of glucose, arabinose, xylose, and ethanol in the fermentation liquid were measured using HPLC (Prominence, available from SHIMADZU CORPORATION) under the following conditions.

### Column: AminexHPX-87H

Mobile phase: 0.01 N H₂SO₄
Flow rate: 0.6 ml/min

### Temperature: 30° C.

Detector: Differential refractive index detector RID-10A

2. Results

2.1. Screening of New araB Gene

The ethanol concentration and the arabinose concentration in the medium were measured for the Uz2861 to 2871 and 2875 strains produced in Chapter 1.10. above, and the increment in ethanol and the decrease in arabinose were calculated. Table 4 shows the results.

TABLE 4

| | | Ethanol | Arabinose | Ethanol | Arabinose |
|---|---|---|---|---|---|
| | | concentration | concentration | increment | decrease |
| Introduced araB | Strain name | (g/L) | (g/L) | (g/L) | (g/L) |
| — | Uz2839 | 9.9 | 37.6 | 0.0 | 0 |
| | (Control strain) | | | | |
| LParaB | Uz2875 | 15.1 | 27.4 | 5.2 | 10.2 |
| LCaraB | Uz2863 | 13.9 | 28.4 | 4.0 | 9.2 |
| LFaraB | Uz2864 | 14.4 | 27.2 | 4.5 | 10.4 |
| LSaraB | Uz2865 | 14.2 | 30.3 | 4.3 | 7.3 |
| PLaraB | Uz2866 | 13.3 | 30.0 | 3.4 | 7.6 |
| TSaraB | Uz2867 | 15.8 | 25.4 | 5.9 | 12.2 |
| CNaraB | Uz2861 | 15.9 | 28.3 | 6.0 | 9.3 |
| SSaraB | Uz2870 | 15.3 | 28.1 | 5.4 | 9.5 |
| PSaraB | Uz2871 | 15.1 | 29.2 | 5.2 | 8.4 |
| BCaraB | Uz2868 | 12.3 | 36.6 | 2.4 | 1 |
| MCaraB | Uz2869 | 16.9 | 26.0 | 7.0 | 11.6 |

The results shown in Table 4 were obtained with a medium composition of glucose: 30 g/L, arabinose: 40 g/L, and yeast extract: 10 g/L at a fermentation temperature of 31° C. Further, the concentration of each substance was an average of measured values for three recombinant strains that were independently obtained with a fermentation time of 48 hours.

As shown in Table 4, in many of the Uz2861 to Uz2871 strains with a new araB gene introduced, the amount of ethanol produced was significantly lower than in the Uz2875 strain with a known araB gene derived from Lactobacillus plantarum introduced. However, it was revealed that the Uz2867 strain, the Uz2861 strain, the Uz2870 strain, the Uz2871 strain, and the Uz2869 strain showed an ethanol productivity and/or an arabinose assimilation superior to those of the Uz2875 strain in which a known araB gene was introduced, as shown in Table 4.

From these results, it was revealed that the araB gene of Thermoactinomyces sp. (NCBI Accession number: WP_049720024, SEQ ID NO: 7 and 8), the araB gene of Clostridium nexile (NCBI Accession number: CDC22812, SEQ ID NO: 9 and 10), the araB gene of Selenomonas sp. oral taxon (NCBI Accession number: WP_050342034, SEQ ID NO: 11 and 12), the araB gene of Paenibacillus sp. (NCBI Accession number: WP_039877980, SEQ ID NO: 13 and 14), and the araB gene of Megasphaera cerevisiae (NCBI Accession number: WP_048515518, SEQ ID NO: 15 and 16) encode L-ribulokinase capable of achieving excellent ethanol productivity and/or excellent arabinose assimilation when imparting the ability to metabolize arabinose to the yeast.

2.2. Screening of New araD Gene

The ethanol concentration and the arabinose concentration in the medium were measured for the Uz3003, 3010, 3011, and 3121 to 3129 strains produced in Chapter 1.12. above, to calculate the increment in ethanol and the decrease in arabinose. Table 5 shows the results.

TABLE 5

| | | Ethanol | Arabinose | Ethanol | Arabinose |
|---|---|---|---|---|---|
| | | concentration | concentration | increment | decrease |
| Introduced araD | Strain name | (g/L) | (g/L) | (g/L) | (g/L) |
| — | Uz2943 | 16.0 | 38.8 | 0.0 | 0.0 |
| | (Control strain) | | | | |
| LParaD | Uz3011 | 25.0 | 18.6 | 9.0 | 20.2 |
| BLaraD1 | Uz3003 | 25.1 | 18.5 | 9.1 | 20.3 |
| BLaraD2 | Uz3010 | 17.1 | 37.0 | 1.1 | 1.8 |
| AHaraD | Uz3121 | 24.5 | 18.3 | 8.5 | 20.5 |
| AParaD | Uz3122 | 25.1 | 17.9 | 9.2 | 20.9 |
| BAaraD | Uz3123 | 24.7 | 18.3 | 8.7 | 20.5 |
| CS17araD | Uz3124 | 26.0 | 14.0 | 10.0 | 24.8 |
| FParaD | Uz3126 | 10.8 | 36.0 | 0.2 | 2.8 |
| LlaraD | Uz3127 | 24.0 | 5.4 | 8.0 | 33.4 |
| MSaraD | Uz3128 | 24.4 | 5.3 | 8.4 | 33.5 |
| SSaraD | Uz3129 | 14.0 | 28.9 | 0.8 | 9.9 |

The results shown in Table 5 were obtained with a medium composition of glucose: 30 g/L, arabinose: 40 g/L, and yeast extract: 10 g/L at a fermentation temperature of 31° C. Further, the concentration of each substance was an average of measured values for three to five recombinant strains that were independently obtained with a fermentation time of 48 hours.

As shown in Table 5, in some of the Uz3003 strain, the UZ3010, and the Uz3121 to 3129 strains with a new araD gene introduced, the amount of ethanol produced was significantly lower than in the Uz3011 strain with a known araD gene derived from Lactobacillus plantarum introduced, and the amount of ethanol produced did not increase, though the arabinose assimilation was excellent. However, it was revealed that the Uz3003 strain, the Uz3122 strain, and the Uz3124 strain showed an ethanol productivity and/or an arabinose assimilation superior to those of the Uz3011 strain with a known araD gene introduced, as shown in Table 5.

From these results, it was revealed that the araD gene of Bacillus licheniformis (NCBI Accession number: WP_003182291, SEQ ID NO: 17 and 18), the araD gene of Alkalibacterium putridalgicola (NCBI Accession number: WP_091486828, SEQ ID NO: 19 and 20), and the araD gene of Carnobacterium sp. 17-4 (NCBI Accession number: WP_013709965, SEQ ID NO: 21 and 22) encode L-ribulose-5-phosphate-4-epimerase capable of achieving excellent ethanol productivity and/or excellent arabinose assimilation when imparting the ability to metabolize arabinose to the yeast.

In particular, although a BLaraD1 gene (accession number: WP_003182291) and a BLaraD2 gene (accession number: WP_011198185) were mentioned as candidates of the araD gene derived from Bacillus licheniformis, in this example, it was revealed that only the BLaraD1 gene (accession number: WP_003182291) functioned in yeasts, whereas the BLaraD2 gene (accession number: WP_011198185) did not function.

2.3. Screening of New araA Gene

The ethanol concentration and the arabinose concentration in the medium were measured for the Uz3181 to 3194 strains produced in Chapter 1.14. above, to calculate the increment in ethanol and the decrease in arabinose. Table 6 shows the results.

TABLE 6

| | | Ethanol | Arabinose | Ethanol | Arabinose |
|---|---|---|---|---|---|
| | Strain | concentration | concentration | increment | decrease |
| Introduced araA | name | (g/L) | (g/L) | (g/L) | (g/L) |
| — | Uz3151 | 10.5 | 39.7 | 0.0 | 0.0 |
| | (Control strain) | | | | |
| LParaA | Uz3181 | 17.9 | 23.5 | 7.5 | 16.1 |
| CAaraA1 | Uz3184 | 21.5 | 13.4 | 11.1 | 26.2 |
| BLaraA2 | Uz3010 | 20.6 | 16.1 | 10.1 | 23.5 |
| SRaraA | Uz3188 | 19.8 | 18.7 | 9.3 | 21.0 |
| LLaraA | Uz3191 | 17.9 | 23.0 | 7.5 | 16.6 |
| MCaraA | Uz3193 | 17.9 | 23.0 | 7.5 | 16.6 |
| OOaraA | Uz3194 | 15.7 | 31.1 | 5.3 | 8.6 |
| BLaraA1 | Uz3183 | 17.0 | 35.2 | 6.5 | 4.4 |
| CAaraA2 | Uz3186 | 19.0 | 32.7 | 9.0 | 7.0 |
| BAaraA | Uz3189 | 16.0 | 38.7 | 5.5 | 1.0 |
| BHaraA | Uz3190 | 16.8 | 37.4 | 6.3 | 2.3 |
| LSaraA | Uz3192 | 20.7 | 28.6 | 10.2 | 11.1 |

The results shown in Table 6 were obtained with a medium composition of glucose: 30 g/L, arabinose: 40 g/L, and yeast extract: 10 g/L at a fermentation temperature of 31° C. Further, the concentration of each substance was an average of measured values for three recombinant strains that were independently obtained with a fermentation time of 24 hours.

As shown in Table 6, in some of the Uz3181 to 3194 strains produced in Chapter 1.14. above, the amount of ethanol produced was significantly lower than in the Uz3181 strain with a known araA gene derived from Lactobacillus plantarum introduced, the amount of ethanol produced did not increase, though the arabinose assimilation was excellent, and the arabinose assimilation was poor. However, it was revealed that, of the Uz3181 to 3194 strains produced in Chapter 1.14. above, the Uz3010 strain, the Uz3188 strain, and the Uz3192 strain with a new araA gene introduced showed an ethanol productivity and/or an arabinose assimilation superior to those of the Uz3181 strain with a known araA gene introduced, as shown in Table 6. The araA gene introduced into the Uz3184 strain and the Uz3186 strain is known in U.S. Pat. No. 8,753,862 B2.

From these results, it was revealed that the araA gene of Bacillus licheniformis (NCBI Accession number: WP_011198012, SEQ ID NO: 1 and 2), the araA gene of Selenomonas ruminantium (NCBI Accession number: WP_072306024, SEQ ID NO: 3 and 4) and the araA gene of Lactobacillus sakei (NCBI Accession number: WP_011375537, SEQ ID NO: 5 and 6) encode L-arabinose isomerase capable of achieving excellent ethanol productivity and/or excellent arabinose assimilation when imparting the ability to metabolize arabinose to the yeast.

2.4. Combination of Xylose Assimilation Technique and Arabinose Assimilation Technique

The ethanol concentration, the arabinose concentration, and the xylose concentration in the medium were measured for the Uz3380 strain and the Uz3381 strain obtained by introducing arabinose assimilation genes (GAL2, araA, araB, and araD genes) into a strain having the ability to assimilate xylose, which was produced in Chapter 1.15. above, to calculate the increment in ethanol and the decrease in arabinose. Table 7 shows the results.

TABLE 7

| | | Ethanol | Arabinose | Xylose | Ethanol | Arabinose |
|---|---|---|---|---|---|---|
| Introduced | Strain | concentration | concentration | concentration | increment | decrease |
| araA | name | (g/L) | (g/L) | (g/L) | (g/L) | (g/L) |
| — | Uz3337 | 32.7 | 18.5 | 6.9 | 0.0 | 0.0 |
| | (Control | | | | | |
| | strain) | | | | | |
| BLaraA2 | Uz3380 | 36.8 | 9.5 | 7.7 | 4.1 | 9.1 |
| SRaraA | Uz3381 | 37.0 | 8.3 | 8.1 | 4.3 | 10.2 |

The results shown in Table 7 were obtained with a medium composition of glucose 60 g/L, xylose 20 g/L, arabinose 20 g/L, and yeast extract 10 g/L at a fermentation temperature of 31° C. Further, the concentration of each substance was an average of measured values for four recombinant strains that were independently obtained with a fermentation time of 48 hours.

As shown in Table 7, it was revealed that the Uz3380 strain and the Uz3381 strain produced in Chapter 1.15. above metabolized glucose, xylose, and arabinose and produced ethanol at a high level.

All publications, patents and patent applications cited in this specification are incorporated in this specification by reference in their entirety.

1. A recombinant yeast comprising a group L-arabinose metabolic genes including an L-arabinose isomerase gene, an L-ribulokinase gene, and an L-ribulose-5-phosphate-4-epimerase gene introduced thereinto, wherein
the L-arabinose isomerase gene is a gene encoding any one of proteins (a) to (c) below:
(a) a protein comprising one amino acid sequence selected from the group consisting of SEQ ID NOs: 2, 4, and 6;
(b) a protein comprising an amino acid sequence having an identity of 80% or more to one amino acid sequence selected from the group consisting of SEQ ID NOs: 2, 4, and 6 and having L-arabinose isomerase activity; and
(c) a protein encoded by a nucleotide sequence that hybridizes with a nucleotide sequence complementary to one nucleotide sequence selected from the group consisting of SEQ ID NOs: 1, 3, and 5 under stringent conditions and having L-arabinose isomerase activity.

2. A recombinant yeast comprising a group of L-arabinose metabolic genes including an L-arabinose isomerase gene, an L-ribulokinase gene, and an L-ribulose-5-phosphate-4-epimerase gene introduced thereinto, wherein
the L-ribulokinase gene is a gene encoding any one of proteins (a) to (c) below:
(a) a protein comprising one amino acid sequence selected from the group consisting of SEQ ID NOs: 8, 10, 12, 14, and 16;
(b) a protein comprising an amino acid sequence having an identity of 80% or more to one amino acid sequence selected from the group consisting of SEQ ID NOs: 8, 10, 12, 14, and 16 and having L-ribulokinase activity; and
(c) a protein encoded by a nucleotide sequence that hybridizes with a nucleotide sequence complementary to one nucleotide sequence selected from the group consisting of SEQ ID NOs: 7, 9, 11, 13, and 15 under stringent conditions and having L-ribulokinase activity.

3. A recombinant yeast comprising a group of L-arabinose metabolic genes including an L-arabinose isomerase gene, an L-ribulokinase gene, and an L-ribulose-5-phosphate-4-epimerase gene introduced thereinto, wherein
the L-ribulose-5-phosphate-4-epimerase gene is a gene encoding any one of proteins (a) to (c) below:
(a) a protein comprising one amino acid sequence selected from the group consisting of SEQ ID NOs: 18, 20, and 22;
(b) a protein comprising an amino acid sequence having an identity of 80% or more to one amino acid sequence selected from the group consisting of SEQ ID NOs: 18, 20, and 22 and having L-ribulose-5-phosphate-4-epimerase activity; and
(c) a protein encoded by a nucleotide sequence that hybridizes with a nucleotide sequence complementary to one nucleotide sequence selected from the group consisting of SEQ ID NOs: 17, 19, and 21 under stringent conditions and having L-ribulose-5-phosphate-4-epimerase activity.

4. The recombinant yeast according to claim 1, which overexpresses a galactose permease gene.

5. The recombinant yeast according to claim 4, wherein
the galactose permease gene is a gene encoding any one of proteins (a) to (c) below:
(a) a protein comprising the amino acid sequence of SEQ ID NO: 24;
(b) a protein comprising an amino acid sequence with an identity of 80% or more to the amino acid sequence of SEQ ID NO: 24 and having galactose permease activity; and
(c) a protein encoded by a nucleotide sequence that hybridizes with a nucleotide sequence complementary to the nucleotide sequence of SEQ ID NO: 23 under stringent conditions and having galactose permease activity.

6. The recombinant yeast according to claim 1, wherein a xylose isomerase gene is introduced.

7. The recombinant yeast according to claim 6, wherein
the xylose isomerase gene is a gene encoding any one of proteins (a) to (c) below:
(a) a protein comprising the amino acid sequence of SEQ ID NO: 26;
(b) a protein comprising an amino acid sequence having an identity of 80% or more to the amino acid sequence of SEQ ID NO: 26 and having xylose isomerase activity; and
(c) a protein encoded by a nucleotide sequence that hybridizes with a nucleotide sequence complementary to the nucleotide sequence of SEQ ID NO: 25 under stringent conditions and having xylose isomerase activity.

8. A method for producing ethanol, comprising
a step of culturing the recombinant yeast according to claim 1 in a medium comprising arabinose for ethanol fermentation.

9. The method for producing ethanol according to claim 8, wherein
the medium comprises cellulose, and
at least saccharification of the cellulose simultaneously proceeds with the ethanol fermentation.


SEQUENCE LISTING (286 sequences)

SEQ ID NO: 1 - DNA, 1425 bp, Bacillus licheniformis
  CDS (1)..(1425)
atgttgaccactggtaagaaggagttttggtttgtagttggctctcaacatttgtatggtgaagaaacattagctgaagttagagcacatgcacaagccatgaccgatgctttgaatgaaagtgcagttcttccttatccgttggttttacaggatttggcggttaatgctgacaaaataacatccatcatgaaggaagtcaactacagagatgaagtggctggcgtaattacgtggatgcatacgttttcaccagctaaaatgtggattagaggtaccaaacttctgcaaaagcccctgttacatcttgcaactcagttcaatgaatcgattccttggccgacaatagacatggactttatgaacctaaaccaatctgctcatggtgacagagaatatggctttataaatgccagattgaagaagcaaaacaaagtagtcgtgggttattgggaaagaccagaagtccaacaacagattgccgagtggatggatgtagctgtcgcctataacgagagcttcaacatcaaggtggcaaggtttggtgataacatgcgtaatgtggctgtcactgaaggtgataaaattgaggcgcaaatacagtttggttggactgtagactacttcggtattggggatttagttcagtatgtaaatgctgtcacagacgaggaaatcaataggttatttgccgagtatgcggatttgtacgaattcgattatggcacctattctagagaagattgggagaaatccgttaaagttcaagcatcttatgaaatcgccattaagagattcttggacgatggtggctacaatgccttcactacgaattttgaggatctttacgggatgaaacagttacctggtttggcagttcaacgtttgatggctcaaggttatggttttgcgggagaaggagattggaaaactgcagctttggatagactgctgaaagtcatgtctaggaatcaatcgacaggttttatggaggactacacatacgaattagcagccggacaagaaagtatactacaatcccacatgttggaagtagatccctcattagcgtctaacaaacccaaaattatagtttcacctttaggaattggcgacagagaggatccagctaggttggttttcgatgggaaagcaggagatggcgtagtggttagtatggctgactttgggactcactacaagctactgatcaacgaagtttcagccttcgaacctactgttccagctccaaatctaccagttgcaagagtcttatgggaagttaagccaaattttcaagacggagttaaagcatggctagaaaatggaggtggtcaccatactgtggtgagcttgttcctaacaaccgatcagatgattacatatgctaagttagtagatcttgaatacgtcgtgatcaaataa

SEQ ID NO: 2 - protein, 474 aa, Bacillus licheniformis
MLTTGKKEFWFVVGSQHLYGEETLAEVRAHAQAMTDALNESAVLPYPLVLQDLAVNADKITSIMKEVNYRDEVAGVITWMHTFSPAKMWIRGTKLLQKPLLHLATQFNESIPWPTIDMDFMNLNQSAHGDREYGFINARLKKQNKVVVGYWERPEVQQQIAEWMDVAVAYNESFNIKVARFGDNMRNVAVTEGDKIEAQIQFGWTVDYFGIGDLVQYVNAVTDEEINRLFAEYADLYEFDYGTYSREDWEKSVKVQASYEIAIKRFLDDGGYNAFTTNFEDLYGMKQLPGLAVQRLMAQGYGFAGEGDWKTAALDRLLKVMSRNQSTGFMEDYTYELAAGQESILQSHMLEVDPSLASNKPKIIVSPLGIGDREDPARLVFDGKAGDGVVVSMADFGTHYKLLINEVSAFEPTVPAPNLPVARVLWEVKPNFQDGVKAWLENGGGHHTVVSLFLTTDQMITYAKLVDLEYVVIK

SEQ ID NO: 3 - DNA, 1425 bp, Selenomonas ruminantium
  CDS (1)..(1425)
atgttggaagtcaagaattacgaattctggtttgtggccggttctcaacacttgtacggggaggaacagctgaagaatgtggaaagagatacccaggatatcgtagataaattgaacgcctctggtaagttgccatacaaagttgtctgtaagggggttatgacaactgctgacggtataacacaatttatgaaggatgtaaactatcacgacgaagtggctgggatcattacctggatgcacactttctccccagccaagaattggataaggggtacgaaattattgcagaagccattactgcatttggctacacaatatctcaacgagatcccatacgctacgatagattttgattatatgaacctaaaccagtcagcccatggtgatagagaatacggttaccttaacagtagacttaacatcaacaacaaaatcgtttatggctactggggtgatgaagaagtccagaaggagatcgctgactggcaagacgtagctgtcgcttataatgagtctttcaacattagagtctgtaggttcggtgacactatgagggacgtagctgtgacagaaggtgacaaggtagaagcacagattcaacttggttggacggtcgactattggccagtgggagagttggtggatgaagtggacgctgtctctgaagctgatatagatgctgaatacaagaaattgagtgaaatgtacaccttgaatccacaggacaacccacaagataaattcgaagcctctgttagatatcaattaagagaatacattgccattaagaaattcctggacgacaggggttacaacgctttctgtactaactttcaagatcttaagggtctgaaacaactgccaggtttagcagctcaaatgttgatgagagatggttacggtttcgccgcggagggggattggaagcatgctgctctgacgaggttattaaaaattatggctcacaacgacagaaccgtattcatggaagactatactttggatctaagaaaggggcacgaggccattctgggtgcccatatgctagaagtagacccaacgatagcttcggacaagccaaaggtggaagtacatccgttagatattggtggcaaggacgatccagctaggctggtgttcacgggggcggatggtgaagctgtagatgtgacagtggctgacttcagagatggtttcagaatgatctcgtatccagtcgactgtagaaaggctgaagctgagacgccacaccttccagtcgccaaacagctgtggaccccgaagtgcggtttaaagaaaggttcaacagagtggattaaagctggtggaggtcatcatactgtcttgtccttcagactgacagaagaacagattcacgacttaggagtcatgcttggtgtcaacttggttgaaatctgctga

SEQ ID NO: 4 - protein, 474 aa, Selenomonas ruminantium
MLEVKNYEFWFVAGSQHLYGEEQLKNVERDTQDIVDKLNASGKLPYKVVCKGVMTTADGITQFMKDVNYHDEVAGIITWMHTFSPAKNWIRGTKLLQKPLLHLATQYLNEIPYATIDFDYMNLNQSAHGDREYGYLNSRLNINNKIVYGYWGDEEVQKEIADWQDVAVAYNESFNIRVCRFGDTMRDVAVTEGDKVEAQIQLGWTVDYWPVGELVDEVDAVSEADIDAEYKKLSEMYTLNPQDNPQDKFEASVRYQLREYIAIKKFLDDRGYNAFCTNFQDLKGLKQLPGLAAQMLMRDGYGFAAEGDWKHAALTRLLKIMAHNDRTVFMEDYTLDLRKGHEAILGAHMLEVDPTIASDKPKVEVHPLDIGGKDDPARLVFTGADGEAVDVTVADFRDGFRMISYPVDCRKAEAETPHLPVAKQLWTPKCGLKKGSTEWIKAGGGHHTVLSFRLTEEQIHDLGVMLGVNLVEIC

SEQ ID NO: 5 - DNA, 1425 bp, Lactobacillus sakei
  CDS (1)..(1425)
atgcttaatacggaaaactacgagttctggtttgttactggttcacagtctctctacggtgaagaaacgctaaggtccgtggaaaaagacgctaaggaaattgtggagaagctgaacgctagtcatcagttgccatacccaatcgtctttaagttggtagctacgactgctgataacatcacgaaggttatgaaggaagctaactacaacgaccacgtggctggtgttattacgtggatgcatacgttttctccagctaaaaactggatcaggggtacgaagttgctacaaaagccgcttctgcacttggctacccaatttttaaacaagataccgtacgatactatagattttgattatatgaacctgaaccaatcagctcatggcgacagagaatacgcttttataaacgctagactaaggaaaaataacaagattatttcgggttattggggtgacgaagatgtccagaaggctatggccaagtggatggacgtcgctgtagcttacaacgagtcgtttaagataaaggtagttacgtttgctgacaaaatgaggaatgtcgctgttacggacggtgacaaagtggaagctcaaattaaatttggttggacggttgattactggggtgtcggtgatttggtggctgaagtgaacgctgtatctgaagccgacattgatgctaagtacgctgatctgcaaaaggaatatgacttcgttgaaggtcagaatacgccagaaaaatttgaacataatgtcaaataccagatccgtgaatactttgggctaaagaaatttatggacgacagaggttacacagcttttacgactaacttcgaagatttggtcggtctagaacaacttccaggtttggctgcccaattgctaatggcagagggttacggtttcgctggtgagggtgattggaaaaccgctgccctggatagattactgaaaatcatggctcataatgaaaaaactgtatttatggaggactacacactagatctgaggcaaggacatgaagccatcctgggttcgcatatgttggaagttgacccatctatcgctagcgacaagccgagggtggaggtgcatccactagacatcggtgataaggacgatccagctaggctggtttttactggtatgcaaggtgacgccgtagacgtgactatggctgattacggggacgaatttaagctgatgtcttatgatgttagaggtaacaaaccagaagctgatacgcctcacttgccagtggccaaacaattgtggaccccaaaacaagggctcagagagggtgctgtgggttggctgactgtagggggtgggcatcacactgtcttgtcatttgctgtggactcagaacagttacaagatttgtctcatctgtttgatctgacgtacgtgaatatcaaatga

SEQ ID NO: 6 - protein, 474 aa, Lactobacillus sakei
MLNTENYEFWFVTGSQSLYGEETLRSVEKDAKEIVEKLNASHQLPYPIVFKLVATTADNITKVMKEANYNDHVAGVITWMHTFSPAKNWIRGTKLLQKPLLHLATQFLNKIPYDTIDFDYMNLNQSAHGDREYAFINARLRKNNKIISGYWGDEDVQKAMAKWMDVAVAYNESFKIKVVTFADKMRNVAVTDGDKVEAQIKFGWTVDYWGVGDLVAEVNAVSEADIDAKYADLQKEYDFVEGQNTPEKFEHNVKYQIREYFGLKKFMDDRGYTAFTTNFEDLVGLEQLPGLAAQLLMAEGYGFAGEGDWKTAALDRLLKIMAHNEKTVFMEDYTLDLRQGHEAILGSHMLEVDPSIASDKPRVEVHPLDIGDKDDPARLVFTGMQGDAVDVTMADYGDEFKLMSYDVRGNKPEADTPHLPVAKQLWTPKQGLREGAVGWLTVGGGHHTVLSFAVDSEQLQDLSHLFDLTYVNIK

SEQ ID NO: 7 - DNA, 1602 bp, Thermoactinomyces sp.
  CDS (1)..(1602)
atgaacttggtgaaagtgtcagaagcgattcagcagggcgatgttactctaggcatagagctaggtagcacacgtatcaaggccgttctagtaacggacgacttccacacgatcgccacgggaagttacgtctgggaaaacaaattcgaaaacggtatctggacatacgctcttgatgaggtttgggaaggaatacagagcagctacgcacaactagcgaaggaggtgaggtccaaatatcaagtgccgcttaccaagatcggtgcaatcggggtcagcgctatgatgcacggatacctagcattctcaaagaatggagatctactgaccccttttaggacctggagaaacaatatcacggagcgtgccgcctcacagcttacggacttgtttcaattcaacattccagaaagatggtcaatagcacatctttaccaagcgattctaaaccaggaacctcatgtaaaggatatacattttatcacaacgcttgcaggttacgtacattggcgtctgagcggcaaaaaagtcttgggtatcggtgatgcaagcggcgttttccctattgacgaagtgacgggaacctattccgccggtctactaaaacgttttgaaagtttggagaacgttgcttcttacccttggaaaattaaagaaattctacctgaagtgttaaaggcgggcgaatgtgcggggcgtcttacaaaagagggtgcccgtttgctggacgctggaggtaaacttcaagaaggttccttaatggtgccacccgagggggatgcgggcacgggcatggtctctacgaacagcgtacgtaaaaggactggcaacattagtgttggcacatcagccttttcaatgatcgtgttgaataagcctctaaagaaagtttaccgtgatgtggatatcgtcatgactcctgacgggactcccgtcgcaatggtgcacacaaataactgctcctcagatatcgatgcgtgggcgaaccttttcaaggaattcgctgaatgcctgggggtggatatcaataccgacaaactatacgagacattgtttcaagcggcagccaaagctgactgcgacgcaggtggtcttgtgaactactcctacttgtcaggtgagaacataactaggatgcctgcaggtagacctctatttgtcaggaatcctgactccacgtttacacttccgaacttcgtgttggcgcaactgtatgcggctctggctccgctaaagatcggcatggacattctgaagaaagaagagcagattgaaaccgaattctttattgcgcaaggcggtttgtttaaaactccaggcatagcacaacaaattcttgccaatgcacttaatacgcctatagcagtaatgagcacggcgggagaaggcggggcgtggggaatggccttgctagctatctacgcgaaaagaaaagatcagtaccaaaatctagaggacttcttggacagggaagtttttgctaatttagagtcaacaatcttaagtccggaaccagaaggtgtaagtggatatgaggcgtttatcgaaaggtatcagaaaggtttaccggtggagtccatggccataaagtgtctgccgtaa

SEQ ID NO: 8 - protein, 533 aa, Thermoactinomyces sp.
MNLVKVSEAIQQGDVTLGIELGSTRIKAVLVTDDFHTIATGSYVWENKFENGIWTYALDEVWEGIQSSYAQLAKEVRSKYQVPLTKIGAIGVSAMMHGYLAFSKNGDLLTPFRTWRNNITERAASQLTDLFQFNIPERWSIAHLYQAILNQEPHVKDIHFITTLAGYVHWRLSGKKVLGIGDASGVFPIDEVTGTYSAGLLKRFESLENVASYPWKIKEILPEVLKAGECAGRLTKEGARLLDAGGKLQEGSLMVPPEGDAGTGMVSTNSVRKRTGNISVGTSAFSMIVLNKPLKKVYRDVDIVMTPDGTPVAMVHTNNCSSDIDAWANLFKEFAECLGVDINTDKLYETLFQAAAKADCDAGGLVNYSYLSGENITRMPAGRPLFVRNPDSTFTLPNFVLAQLYAALAPLKIGMDILKKEEQIETEFFIAQGGLFKTPGIAQQILANALNTPIAVMSTAGEGGAWGMALLAIYAKRKDQYQNLEDFLDREVFANLESTILSPEPEGVSGYEAFIERYQKGLPVESMAIKCLP

SEQ ID NO: 9 - DNA, 1596 bp, Clostridium nexile
  CDS (1)..(1596)
atgggtaacgtaaaggaaacgatcgagtccggtaaggcagtcctggggattgaatttggatcaacaaggatcaaagcggtgttgatcgacgaggaacatgtacctattgcaagcggggattatgcatgggagaatcgtctggacaacgggatatggacttacacgttagaggacatatggaccggcctgagagagagctacgccaaaatggccgctgatgtgaaaggcaagtacaacgttacgatcaagcaattgggcgctatagggttctcagccatgatgcacggctacatggctttcgatcaagagggcaatttacttgttccctttcgtacttggaggaacggcattacggaacaggcggctgaggcacttacggagctatttcaatataatataccacaacgttggagcatcgcccacctgtaccaggctattctaaacaaagaagagcacgtcaaaaaggtggcattttttacaacattaagcggttacatccactggaaactgacaggtcaaaaagttttgggtgtgggtgatgcctctggtatgttcccaatcgatcctaatacaaagaactatgatgagcgtatgatccaaaagtttgacacacttgtcgagggggaaggcttctcatggaagatcgaagaaatattaccgaaggttcttcaatccggcgaacgtgcaggcatgctaacgcaggagggtgcacgtctactagatatctcagggaatttggaatcaggaatcttattatgtccacctgaaggcgacgcgggtactgggatggtagccacaaatagtgtgaggaaaagaactgggaatgttagcgcgggcacatctgtgtttggcatggtagttcttgaaaaagagcttgaaagggtctatcctgagattgatcttgtgaccacgcccgatggatccctggtcggcatggtgcatgcgaataactgtacaagcgatcttaatgcatgggtcggactattccgtgagtttgccgaaagttttggaataaaggttgatatggacagcctttttgggaccctatatcgtaaagcattagagggtgatgatgactgtgggggcctgctgagttacgggtatctgagtggggaaaatattacaggcgtagccgaggggaggcccttgttcgttagatcccctaagtccaactttaacctatcaaacttcatgagagttcatttattcacgtcactttcagcactgaaaatagggatggatatcatgatgaaagaggaacatgtagagatagacacaatactagggcacggtggcctattcaagactaaaggtgtcggccaaaaaatactagctgctgcgataaacgcaccagtctcagtaatggagaccgctggtgagggtggcccctggggtatggcactgttagccgcgtatatgattcaaaaggaagaacaagagtccctgggcgactatctatcaaagaaggttttcagcggtaatgcaggagtatctatggaaccggatgcaaaagatgtggaaggttttgaaaggtttatcgaaagatataagaatgggatagccattgaacaaagcgctttgaagaacctggtttaa

SEQ ID NO: 10 - protein, 531 aa, Clostridium nexile
MGNVKETIESGKAVLGIEFGSTRIKAVLIDEEHVPIASGDYAWENRLDNGIWTYTLEDIWTGLRESYAKMAADVKGKYNVTIKQLGAIGFSAMMHGYMAFDQEGNLLVPFRTWRNGITEQAAEALTELFQYNIPQRWSIAHLYQAILNKEEHVKKVAFFTTLSGYIHWKLTGQKVLGVGDASGMFPIDPNTKNYDERMIQKFDTLVEGEGFSWKIEEILPKVLQSGERAGMLTQEGARLLDISGNLESGILLCPPEGDAGTGMVATNSVRKRTGNVSAGTSVFGMVVLEKELERVYPEIDLVTTPDGSLVGMVHANNCTSDLNAWVGLFREFAESFGIKVDMDSLFGTLYRKALEGDDDCGGLLSYGYLSGENITGVAEGRPLFVRSPKSNFNLSNFMRVHLFTSLSALKIGMDIMMKEEHVEIDTILGHGGLFKTKGVGQKILAAAINAPVSVMETAGEGGPWGMALLAAYMIQKEEQESLGDYLSKKVFSGNAGVSMEPDAKDVEGFERFIERYKNGIAIEQSALKNLV

SEQ ID NO: 11 - DNA, 1605 bp, Selenomonas sp. Oral taxon
  CDS (1)..(1605)
atggacatgaccgccgcggaaatagtcagaaatggtgaggcgttcctaggcatagagttagggagtacgaggataaaggcagtcttaattgatgccaaatgcaatgttattgcacaggggtcatatgcctgggagaatcacttcgacgggatgtattggtcttatcaccaagacgaaatctggcacggactgagatccgcttacaccgacttaagacagtgcgttgaggagaggtgcagggtgagtatacagcacgtggccgcgatgggcatctccggaatgatgcatgggtacttggctttcgatatggctggcgatctactagtgcctttcaggacttggcgtaacaatacgacgggtgaagctgcgaaagagcttaccagggctcttgcctttaacatacccgagcgttggtctgtagcacacttgtatcaggccttactgtctggcgagccacacgtggctcaggttgactatatgaccactttatcagggtacatccactggaaattgtcagggcaaaaggttttgggcattgatgatgcgtcaggcatgtttcctatcgatccggcgacggggacttatgatatggggatgctaaagatcttctctggcctttcagccgctgctgccctacccaaaaatcttgccgacatattacctcgtgtttgcactgctggacagacagcaggaaaattgacccctgagggggctgctttgttggacgagtcaggaaatttaatggcgggatgcccgatgtgtccaccggagggggacgcaggaacgggaatgatagctacaaacagcatccgtgagaggacaggtaacattagtgtagggaccagcatcttcagtatgaatgtgcttgagcgtccgtttcagaaggtctatcgtgacgttgacatagtaatgacaccacatgggaaacccaccgcgatggttcactcaaataattgtacttctgacataaatgcgtggtttgggatattctctgaaatcgccacggtcctaggcgcctccgttccagccgatactctgtaccaaaagctatttcaacaggtagagcaagctgatccatctggcggcggtttactaaattactcctttctaagcggagagaacttgacgcgtacagaaaagggcagaccattatttgtgcgtacaccgcagtcagcttttaccctggccaattttgtcttgacgcaactgtatgcagcgttcgctcccctgaggatcggttttgacattctaacgcaggaggaaggcataaggatggagagaatgatcgcccacggagggttgttcaggacaccctacatagctcagcaggtgttggccaatatgctggacgtgtctgtcaccgtaatgaaaacagctgccgagggcggtgcgtggggtatggcggtcttggcggtgtatgcacttcgtgggcgtggagaggacctggctgatttcctggacagagaggtatttgctaccgctgagggagaaacgctggcgcccgatgccgtgggcgtttgcggggctcaggaattcatcgcacgttacagggcggcattatctgtagaaaacacggcgggagacgcgttgacttacaacggctag

SEQ ID NO: 12 - protein, 534 aa, Selenomonas sp. Oral taxon
MDMTAAEIVRNGEAFLGIELGSTRIKAVLIDAKCNVIAQGSYAWENHFDGMYWSYHQDEIWHGLRSAYTDLRQCVEERCRVSIQHVAAMGISGMMHGYLAFDMAGDLLVPFRTWRNNTTGEAAKELTRALAFNIPERWSVAHLYQALLSGEPHVAQVDYMTTLSGYIHWKLSGQKVLGIDDASGMFPIDPATGTYDMGMLKIFSGLSAAAALPKNLADILPRVCTAGQTAGKLTPEGAALLDESGNLMAGCPMCPPEGDAGTGMIATNSIRERTGNISVGTSIFSMNVLERPFQKVYRDVDIVMTPHGKPTAMVHSNNCTSDINAWFGIFSEIATVLGASVPADTLYQKLFQQVEQADPSGGGLLNYSFLSGENLTRTEKGRPLFVRTPQSAFTLANFVLTQLYAAFAPLRIGFDILTQEEGIRMERMIAHGGLFRTPYIAQQVLANMLDVSVTVMKTAAEGGAWGMAVLAVYALRGRGEDLADFLDREVFATAEGETLAPDAVGVCGAQEFIARYRAALSVENTAGDALTYNG

SEQ ID NO: 13 - DNA, 1614 bp, Paenibacillus sp.
  CDS (1)..(1614)
atggatcagaacatccgtcaagcgatccttgaagggggcacgtccttaggtatagaatttggcagtactcgtattaaagcagttcttatagataggaaatttgaaacagtagccagcggctcctacgaatgggagaatcagttagtcgatggatattggacgtatgacttgaatgacataataaaaggtctacagaccgcctacagcgaactgaagcaagaggtgcaacagaagtatgaggtcactctttccaaagtaggttccatcggcttttcagcgatgatgcatggatacatggcgttcgattcaaaaggagagttgctggttccgttcaggacttggaggaacgccaccactggggccgctgccaaagagctaacggaagcctttcagtttaatattccggagaggtggagcatagcccatttatatcaggcgatacttaacaaagaggagcatgtaccagacgtagatttcgttacgacgttagccggttacattcactggttactgacagggaccaaagcaatcggcatcggggatgcttctggtatctttcccatagatgaagctgcccaggactacaacccagccatggtaaagcagtttgacgaactaatcgcggcaaagggctattcactaaaactaggcgagctgttgcctaaggtatatcatgcaggcgagcaggccggagttcttacggaagcaggggccgccattttagacgtttcaggcgatttacaagcgggaattccactttgccctccagagggagatgctgggacgggaatggtcgccaccaattcagtacgtaaaagaaccgggaacatatccgtaggtacttcagtttttgccatgatagtactagaaaacgatttgagtgcagtctatcctgagattgatatggtgacaaccccggacgggtctccggtaggaatggtccacgcaaacaactgttcttctgacatcaatgcttgggtgggcttatttagagaattcgcagaggcaatgggcttccaggccgacagtgggaagttgttcagcgtgctgtttggaaaagcgttggaggctgacgcagatgggggtggccttctatcatacggatacttcagcggtgaaaacataacaggtattgaaaagggcagaccgctatttgtgagaagccctgaatctcgttttaaccttgcaaacttcatgagaacacatttatttacggcttttgcagcactaaaaattggaatggacatcctaactaagaaggaaaacgtggccatagatagcatactggctcacggtggcttattcaaaacgcccgtagtgggacagaaaatcgtagctgcaagcatgaacgtgccggtcagtgtgatggccaccgccagtgaaggtggagcatggggtatggctatcctagctgcgtttatgcaaaacagggagcagcaggagcgtctagacgatttcctggacaataaagtatttaaggacaccgaaggtcaggtgatacaccctgatgcagcggatgtcaagggatttgagctattcatggagaggtacgtagaaggattagccatcgagcaggcggcggtagatcatttaatagaaaacgggagggggtag

SEQ ID NO: 14 - protein, 537 aa, Paenibacillus sp.
MDQNIRQAILEGGTSLGIEFGSTRIKAVLIDRKFETVASGSYEWENQLVDGYWTYDLNDIIKGLQTAYSELKQEVQQKYEVTLSKVGSIGFSAMMHGYMAFDSKGELLVPFRTWRNATTGAAAKELTEAFQFNIPERWSIAHLYQAILNKEEHVPDVDFVTTLAGYIHWLLTGTKAIGIGDASGIFPIDEAAQDYNPAMVKQFDELIAAKGYSLKLGELLPKVYHAGEQAGVLTEAGAAILDVSGDLQAGIPLCPPEGDAGTGMVATNSVRKRTGNISVGTSVFAMIVLENDLSAVYPEIDMVTTPDGSPVGMVHANNCSSDINAWVGLFREFAEAMGFQADSGKLFSVLFGKALEADADGGGLLSYGYFSGENITGIEKGRPLFVRSPESRFNLANFMRTHLFTAFAALKIGMDILTKKENVAIDSILAHGGLFKTPVVGQKIVAASMNVPVSVMATASEGGAWGMAILAAFMQNREQQERLDDFLDNKVFKDTEGQVIHPDAADVKGFELFMERYVEGLAIEQAAVDHLIENGRG

SEQ ID NO: 15 - DNA, 1605 bp, Megasphaera cerevisiae
  CDS (1)..(1605)
atgggtttgatgaatgtcgctgccgatattaagaacggagacgtggtgcttggtattgagcttggtagcactagaattaaggctgtcttggttttgaatgattgccacactattgcttccggtagttacgggtgggagaataccctacaagatggcatatggacatactcacttgaagaggtgtggaaaggaattcagatatgttactctcagattgcggccgacgttcaatctaggtatcatatgcctctaacgaggatcagcgccattggagtcagcgcaatgatgcacggctacctggctttcgacggacatgaccgtcttctagtcccttttaggacttggcgtaacaacataaccggaagggcggcagccgatttaacagagaagctacactttaatattccccaaagatggagcatcgcgcacttgtaccaagcgatccttaacaaagagcatcatgtaagtgacataagttacctaacaacgcttgcgggctatgtgcactggcgtctatgcggacgtagagtgcttggcataggcgatgccagcggaatgttcccaatcgatgagaggtcaggaacatatcatgcgggtatgttgaacattttcaatggtttgcccgaagtccagccgtactcatggagactagagaatttgttgccgtcagtcttgagagctggtgaatacgctggtaccctaacagccgaaggtgcgtcacttctggatacagccgggaggttgattcctgggtcagcaatggcgcctcccgagggcgacgcggggaccggcatggtatcaacaaacagtgtaaggaagagaacaggaaacatcagtgttggcacgagcgcgttcagcatgaatgtactggacgggcccctgaaaaacatacactctgatatagatgttgtcaccacacctgaaggggcccccgcggcgatggttcatacgaacaactgtagcagcgacatcaacgcgtggattcagctattcgctgacttcgccgcctgcgccggaatgcagttagccccagatatgctgtatgagttactgttcagccaggctgggagagctgacaccgatgcaggcggacttgtcaattatagctacttatcaggagaaaacgtaacttgcatagacagaggcagacctctatttgtccgtacccccaattcccgtttcacgttacccaattttatgcttacccaattgtatgccgcatttgctcctcttaaactaggcatggacattttaatcagggaagagggcattcaaaccgaggtaatgatagctcagggcggcttattcaaaactcctgtgatcgggcagcaagttcttgcggatgctttgaatatgcccataacgattatggaaaccgcaggtgagggggggccatgggggatggcagtgctaacggttttcatgcaatcttgtgcgcccggcgaaacattagctgattttctggacgaaagggtgttccgtcacccacgtagtactacagtgactccgaattccgaaggtgtagccggctatgcagcattcatagagtcatacacgagagccctgccggttgaagagttagctggtacggcattagcagagtag

SEQ ID NO: 16 - protein, 534 aa, Megasphaera cerevisiae
MGLMNVAADIKNGDVVLGIELGSTRIKAVLVLNDCHTIASGSYGWENTLQDGIWTYSLEEVWKGIQICYSQIAADVQSRYHMPLTRISAIGVSAMMHGYLAFDGHDRLLVPFRTWRNNITGRAAADLTEKLHFNIPQRWSIAHLYQAILNKEHHVSDISYLTTLAGYVHWRLCGRRVLGIGDASGMFPIDERSGTYHAGMLNIFNGLPEVQPYSWRLENLLPSVLRAGEYAGTLTAEGASLLDTAGRLIPGSAMAPPEGDAGTGMVSTNSVRKRTGNISVGTSAFSMNVLDGPLKNIHSDIDVVTTPEGAPAAMVHTNNCSSDINAWIQLFADFAACAGMQLAPDMLYELLFSQAGRADTDAGGLVNYSYLSGENVTCIDRGRPLFVRTPNSRFTLPNFMLTQLYAAFAPLKLGMDILIREEGIQTEVMIAQGGLFKTPVIGQQVLADALNMPITIMETAGEGGPWGMAVLTVFMQSCAPGETLADFLDERVFRHPRSTTVTPNSEGVAGYAAFIESYTRALPVEELAGTALAE

SEQ ID NO: 17 - DNA, 696 bp, Bacillus licheniformis
  CDS (1)..(696)
atgttggagcagttaaaggaagaagtcttacaagcaaaccttgatctacccaaatacggtttggtcaagtatacctggggtaatgcatcagctattgatagagagactgggttattcgtcatcaagccatctggagttgactacgaaaccatgaaggcatcagacatggtagttgttgacttagatggcaatgttgtggaaggcgagttaagacctagctccgatactgccacacatgcagtgttgtacaaaaggtatccggaaataggcggtattgttcacacacattccacatgggctacgatatgggcacaagctggattggatgtaccagctatgggtactactcatgccgatactttctacggatctgttccatgtgcgagatatctgacacaagaggaaatcgatagaggctatgaagctgaaacagggcatgttattatcgagacttttgaggaaagaggtttggatattttggctgttcctggagtgttactacatggtcatggtccatttacgtggggtaaagacgtcaaaagtgcggtgttaaactcggtagtactagaagaagtcgctaaaatgaacttgtttaccagagaactgaatctgtttgccaaagaacttcctcaaaggatacttgacaagcactatttgcgtaaacacggtaagaatgcctattacggtcagaaataa

SEQ ID NO: 18 - protein, 231 aa, Bacillus licheniformis
MLEQLKEEVLQANLDLPKYGLVKYTWGNASAIDRETGLFVIKPSGVDYETMKASDMVVVDLDGNVVEGELRPSSDTATHAVLYKRYPEIGGIVHTHSTWATIWAQAGLDVPAMGTTHADTFYGSVPCARYLTQEEIDRGYEAETGHVIIETFEERGLDILAVPGVLLHGHGPFTWGKDVKSAVLNSVVLEEVAKMNLFTRELNLFAKELPQRILDKHYLRKHGKNAYYGQK

SEQ ID NO: 19 - DNA, 693 bp, Alkalibacterium putridalgicola
  CDS (1)..(693)
atgctagaaaagttaaagcaggacgtctatgaagctaacatggcacttccaaaacatggtctggtcgtcttcacctggggtaatgtatcaggtgttgatagggaaaaagaactggtggttatcaaaccgtctggtgtggactatgatgaattatcaccagaaaatatggtagtggtagatttcgatggtaatttagttgaaggtgacctcaagccatcatctgacactgctacgcacattgagctttataaaaactttaaggacatcggtggtgtggtgcacactcactctccctgggctgtatccttcgcccaagctggcgtggacctgccagctgctggtactacgcatgctgactatttttacggtgatattcctgttacgagagctatgaaacaagctgaaattgaaactgagtacgaaaaggaaacggggagtgtaattgtcgaaacattcaaggaaaggaacattgaaccgaatcagataccaggtgtcctggtaaatgatcacggtccgtttacatggggtactgacccggccaatgctgtgtacaattctgtagttttagaacaagtagctcaaatgacataccatggtatgcaattgaagggtgaatccgtcagaatggaccagacacttctcgacaagcactatctgagaaagcacggtgagaatgcttactacggtcaatga

SEQ ID NO: 20 - protein, 230 aa, Alkalibacterium putridalgicola
MLEKLKQDVYEANMALPKHGLVVFTWGNVSGVDREKELVVIKPSGVDYDELSPENMVVVDFDGNLVEGDLKPSSDTATHIELYKNFKDIGGVVHTHSPWAVSFAQAGVDLPAAGTTHADYFYGDIPVTRAMKQAEIETEYEKETGSVIVETFKERNIEPNQIPGVLVNDHGPFTWGTDPANAVYNSVVLEQVAQMTYHGMQLKGESVRMDQTLLDKHYLRKHGENAYYGQ

SEQ ID NO: 21 - DNA, 705 bp, Carnobacterium sp. 17-4
  CDS (1)..(705)
atgctggaacaacttaaggaggaagttttccaggctaatctcgaacttcccaagcaaggtctaattaagtacacgtggggtaatgtctcagctgtggatagggaacagggtctgttcgtgattaagccgtctggtatctcatatgaagaaatgaaggcttccgatatggtagtctgtgacatggatggtaaagtcgtggaaggtaagctaaacccgtctagcgatacgctcacccacgccgtgctgtataaggagttttccgaaattggtggtattgtgcatacacatagtacatgggccacgatctgggctcaagccgggctggacgtcccggccatgggtacaacccatgcggatacattctatggtagtattccatgtacgaggtttttgaatcagaaggaaatcgatgaaggttatgaggaacagactggtcatgtaatcgttgaaactttcaaggaaaggaatatcaaacctatggagatcccgggagctttattgcacggccatggtccgtttacctggggtaaaaacgccaaagaagctgtcatgaacgctgtcgttttagacgaagtgtgtaaaatgaatctgttctctagacagcttaattccttcagtgaagaattgccgcagagggttctagacaaacattatttgaggaaacacggtgaaaatgcttactacggtcaaaaaaataagcactga

SEQ ID NO: 22 - protein, 234 aa, Carnobacterium sp. 17-4
MLEQLKEEVFQANLELPKQGLIKYTWGNVSAVDREQGLFVIKPSGISYEEMKASDMVVCDMDGKVVEGKLNPSSDTLTHAVLYKEFSEIGGIVHTHSTWATIWAQAGLDVPAMGTTHADTFYGSIPCTRFLNQKEIDEGYEEQTGHVIVETFKERNIKPMEIPGALLHGHGPFTWGKNAKEAVMNAVVLDEVCKMNLFSRQLNSFSEELPQRVLDKHYLRKHGENAYYGQKNKH

SEQ ID NO: 23 - DNA, 1725 bp, Saccharomyces cerevisiae
  CDS (1)..(1725)
atggcagttgaggagaacaatatgcctgttgtttcacagcaaccccaagctggtgaagacgtgatctcttcactcagtaaagattcccatttaagcgcacaatctcaaaagtattctaatgatgaattgaaagccggtgagtcagggtctgaaggctcccaaagtgttcctatagagatacccaagaagcccatgtctgaatatgttaccgtttccttgctttgtttgtgtgttgccttcggcggcttcatgtttggctgggataccggtactatttctgggtttgttgtccaaacagactttttgagaaggtttggtatgaaacataaggatggtacccactatttgtcaaacgtcagaacaggtttaatcgtcgccattttcaatattggctgtgcctttggtggtattatactttccaaaggtggagatatgtatggccgtaaaaagggtctttcgattgtcgtctcggtttatatagttggtattatcattcaaattgcctctatcaacaagtggtaccaatatttcattggtagaatcatatctggtttgggtgtcggcggcatcgccgtcttatgtcctatgttgatctctgaaattgctccaaagcacttgagaggcacactagtttcttgttatcagctgatgattactgcaggtatctttttgggctactgtactaattacggtacaaagagctattcgaactcagttcaatggagagttccattagggctatgtttcgcttggtcattatttatgattggcgctttgacgttagttcctgaatccccacgttatttatgtgaggtgaataaggtagaagacgccaagcgttccattgctaagtctaacaaggtgtcaccagaggatcctgccgtccaggcagagttagatctgatcatggccggtatagaagctgaaaaactggctggcaatgcgtcctggggggaattattttccaccaagaccaaagtatttcaacgtttgttgatgggtgtgtttgttcaaatgttccaacaattaaccggtaacaattattttttctactacggtaccgttattttcaagtcagttggcctggatgattcctttgaaacatccattgtcattggtgtagtcaactttgcctccactttctttagtttgtggactgtcgaaaacttgggacatcgtaaatgtttacttttgggcgctgccactatgatggcttgtatggtcatctacgcctctgttggtgttactagattatatcctcacggtaaaagccagccatcttctaaaggtgccggtaactgtatgattgtctttacctgtttttatattttctgttatgccacaacctgggcgccagttgcctgggtcatcacagcagaatcattcccactgagagtcaagtcgaaatgtatggcgttggcctctgcttccaattgggtatgggggttcttgattgcatttttcaccccattcatcacatctgccattaacttctactacggttatgtcttcatgggctgtttggttgccatgtttttttatgtctttttctttgttccagaaactaaaggcctatcgttagaagaaattcaagaattatgggaagaaggtgttttaccttggaaatctgaaggctggattccttcatccagaagaggtaataattacgatttagaggatttacaacatgacgacaaaccgtggtacaaggccatgctagaataa

SEQ ID NO: 24 - protein, 574 aa, Saccharomyces cerevisiae
MAVEENNMPVVSQQPQAGEDVISSLSKDSHLSAQSQKYSNDELKAGESGSEGSQSVPIEIPKKPMSEYVTVSLLCLCVAFGGFMFGWDTGTISGFVVQTDFLRRFGMKHKDGTHYLSNVRTGLIVAIFNIGCAFGGIILSKGGDMYGRKKGLSIVVSVYIVGIIIQIASINKWYQYFIGRIISGLGVGGIAVLCPMLISEIAPKHLRGTLVSCYQLMITAGIFLGYCTNYGTKSYSNSVQWRVPLGLCFAWSLFMIGALTLVPESPRYLCEVNKVEDAKRSIAKSNKVSPEDPAVQAELDLIMAGIEAEKLAGNASWGELFSTKTKVFQRLLMGVFVQMFQQLTGNNYFFYYGTVIFKSVGLDDSFETSIVIGVVNFASTFFSLWTVENLGHRKCLLLGAATMMACMVIYASVGVTRLYPHGKSQPSSKGAGNCMIVFTCFYIFCYATTWAPVAWVITAESFPLRVKSKCMALASASNWVWGFLIAFFTPFITSAINFYYGYVFMGCLVAMFFYVFFFVPETKGLSLEEIQELWEEGVLPWKSEGWIPSSRRGNNYDLEDLQHDDKPWYKAMLE

SEQ ID NO: 25 - DNA, 1320 bp, Unknown
  Obtained from Intestinal Protist of Reticulitermes speratus
  CDS (1)..(1320)
atgtctcaaatttttaaggatatcccagttattaaatatgaaggtccagcttccaagaatcctttgagtttcaaatactacgatgcaaacaaggttattgatggtaaaccaatgaaggaacatttgagatacgcaatggcttggtggcataatttgtgtgctaccggtcaagatatgtttggtcctggtactgcagataaatccttcggtagtaagacagttggtaccatggaacatgcacatgctaaagttgatgctggttttgaattcatgtccaagttgggtgttgaatacttctgtttccatgatgctgatttggttccagaagcagatactttgagtgaaacaaacaaaagattggatgaaatcgctgaacatatcgttgctaagcaaaaggcaactggtattaaatgtttgtggggtacagcaaatttgttttctaaccctagattcttaaatggttctggttcttcaaactcagctgatgtttatgcatacgctgcagctcaaattaaaaaggctttggatttgactgttaaatttggtggtgttggttatgttttctggggtggtagagaaggttacgaaaccttgttgaacactgatgttaagttcgaacaagaaaacatcgctaacttgatgcatttggcagttacttacggtagatcaatcggttttaaaggtgacttctacattgaaccaaaacctaaggaaccaacaaagcatcaatatgattttgatgcagctactacaattggtttcattagacaatacggtttggaaaaggatttcaagttgaacatcgaagcaaaccatgctacattagcaggtcataccttccaacatgatttgagaatctctgctattaatggcatgttaggttcagttgatgcaaacacaggtgacccattgttaggttgggataccgatgaatttccttattccgtttacgataccactttggctatgtacgaaattattaaggcaggtggtttgaccggtggtttgaattttgattccaaggttagaagaccaagttacacacatgaagatttgttttacggtttcattttgggtatggattctttcgctttgggtttgattaaagcaaaggctttgattgcagatggtagattggattcattcgttaaggatagatacgcttcttacggttcaggtattggtgctaagattagagatcattctgcaactttggaagaattagcagcttatgcattagctaaagatacagttgctttgcctggttccggtagacaagaatacttagaaagtattattaaccaaattttgtttcaataa

SEQ ID NO: 26 - protein, 439 aa, Unknown
  Synthetic Construct
MSQIFKDIPVIKYEGPASKNPLSFKYYDANKVIDGKPMKEHLRYAMAWWHNLCATGQDMFGPGTADKSFGSKTVGTMEHAHAKVDAGFEFMSKLGVEYFCFHDADLVPEADTLSETNKRLDEIAEHIVAKQKATGIKCLWGTANLFSNPRFLNGSGSSNSADVYAYAAAQIKKALDLTVKFGGVGYVFWGGREGYETLLNTDVKFEQENIANLMHLAVTYGRSIGFKGDFYIEPKPKEPTKHQYDFDAATTIGFIRQYGLEKDFKLNIEANHATLAGHTFQHDLRISAINGMLGSVDANTGDPLLGWDTDEFPYSVYDTTLAMYEIIKAGGLTGGLNFDSKVRRPSYTHEDLFYGFILGMDSFALGLIKAKALIADGRLDSFVKDRYASYGSGIGAKIRDHSATLEELAAYALAKDTVALPGSGRQEYLESIINQILFQ

SEQ ID NO: 27 - DNA, 1602 bp, Lactobacillus plantarum
  CDS (1)..(1602)
atgaatttggtcgaaaccgcacaagccataaaaactggtaaagtttctttgggtatcgaattgggttcaactagaattaaagctgttttgatcacagatgacttcaacaccattgcatccggtagttatgtctgggaaaaccaattcgtagatggtacatggacctacgctttggaagacgtctggaccggtatccaacaatcctatactcaattggctgcagatgttagatctaagtaccatatgtcattgaagcacatcaacgcaatcggtatctctgccatgatgcatggttatttggctttcgatcaacaagcaaagttgttagttccattcagaacatggagaaataacattaccggtcaagccgctgatgaattgacagaattgttcgacttcaacatacctcaaagatggtcaatcgcccatttgtaccaagctatcttgaacaacgaagcccacgtaaagcaagttgattttatcactacattggctggttacgtaacttggaaattgtccggtgaaaaggttttaggtattggtgacgctagtggtgtctttccaatagatgaaaccactgacacatacaaccaaactatgttgacaaaattctcccaattggataaggttaagccttacagttgggacattagacacatattgccaagagtcttacctgctggtgcaattgccggtaaattgactgcagccggtgcatccttgttagatcaaagtggtacattagacgcaggttcagttattgccccacctgaaggtgacgctggtactggtatggttggtacaaattctgtcagaaaaagaactggtaacatttcagtaggtacctctgctttttcaatgaacgttttggataagccattgtctaaggtttacagagatatcgacattgtcatgactccagatggttcacctgttgctatggttcatgtcaataactgttcttcagacatcaacgcttgggcaacaatttttcacgaattcgctgcaagattgggtatggaattgaagccagatagattgtacgaaactttgttcttagaatctacaagagccgatgctgacgcaggtggtttagcaaattattcctaccaaagtggtgaaaacatcaccaaaattcaagctggtagaccattgttcgttagaactcctaactctaagttttcattgccaaacttcatgttgactcaattgtatgccgctttcgcacctttgcaattgggtatggatatcttggttaacgaagaacatgtccaaacagacgtaatgatcgctcaaggtggtttgtttagaaccccagtaattggtcaacaagttttggccaatgctttaaacataccaatcaccgtaatgtctactgcaggtgaaggtggtccttggggtatggcagttttagccaatttcgcttgcagacaaactgctatgaacttggaagatttcttggaccaagaagttttcaaagaaccagaatccatgacattgagtccagaacctgaaagagtcgccggttatagagaattcattcaaagataccaagctggtttacctgttgaagcagccgctggtcaagctattaaatactaa

SEQ ID NO: 28 - protein, 533 aa, Lactobacillus plantarum
MNLVETAQAIKTGKVSLGIELGSTRIKAVLITDDFNTIASGSYVWENQFVDGTWTYALEDVWTGIQQSYTQLAADVRSKYHMSLKHINAIGISAMMHGYLAFDQQAKLLVPFRTWRNNITGQAADELTELFDFNIPQRWSIAHLYQAILNNEAHVKQVDFITTLAGYVTWKLSGEKVLGIGDASGVFPIDETTDTYNQTMLTKFSQLDKVKPYSWDIRHILPRVLPAGAIAGKLTAAGASLLDQSGTLDAGSVIAPPEGDAGTGMVGTNSVRKRTGNISVGTSAFSMNVLDKPLSKVYRDIDIVMTPDGSPVAMVHVNNCSSDINAWATIFHEFAARLGMELKPDRLYETLFLESTRADADAGGLANYSYQSGENITKIQAGRPLFVRTPNSKFSLPNFMLTQLYAAFAPLQLGMDILVNEEHVQTDVMIAQGGLFRTPVIGQQVLANALNIPITVMSTAGEGGPWGMAVLANFACRQTAMNLEDFLDQEVFKEPESMTLSPEPERVAGYREFIQRYQAGLPVEAAAGQAIKY

SEQ ID NO: 29 - DNA, 1611 bp, Lactobacillus composti
  CDS (1)..(1611)
atgaacactctggagatatcaaaggcgatacaggccggcaacgtgtctctaggtatagaattggggtctacccagataaaaagtgtgcttgtaaccgacgatttctctacaatcgcttcagggagctacgtttgggagaacaagtttgaacagggggtttggacgtaccccatcgaagaagtatggaatggtatccaaacttcctatacgcagatggctgctaatgttcagtctaagtaccacgaaaacctagtgcacataagcgcaattggaatcagtgccatgatgcacgggtatctagcgtttgataaatccgataagctgttagtaccgttccgtacatggagaaataacattacggaggaagctgctgataaattgaccgatctatttgatttcaacatcccacaaagatggacgattgcccacctatatcagagcattctaaaccaggaagcacatgtggcggatctagattttgtgactacgctggcagggtacgttacctggaagctgagtggagagaaagtacttggcattggtgacgcttcaggcgtgttccctatagatgaaaccactggaacgtatcgtgcggatctattggacaagttcaaccagctaccagaggtggtccaatacaactggcaaacgacacaactattgcctaaggtagagaaagcgggagtccaggctggcaccttaactgcagaaggagcaaagctactagacgtgaacggcaacttagcggcgggttcactaatggcaccgcctgaaggtgatgctggtaccgggatggtagggaccaactctgtaaggaagaggactggcaacatcagtgtgggcacctcagcatttagcatggttgtgttagataaaccactgaaggcactgcacagagatatcgatatcgtgacaacccctactggcgcccctgtcgcaatggtgcatacgaataattgctccagtgacataaatgcatgggtggcgatcttcaaggagttcgcggcacgtctaggtgtgaatttgcccgcagaccagctgtaccaaactctattcctagaagccactcacgcagacccggacgcaggtggcctgcttaacttctcatatctttcaggtgagaatataactaaaataaaggcagggcgtcccttatttgttagaacgccgaactccaactttaacctgccgaactttattcagactcaactgtacgctgccttcgcgcctctgaaaatcggaatggatatcctagtcaaggacgagcatataaacactgaggccatgatagcccaagggggtctgtttaagacgccagtcataggacaacaggttctagccaatgcgctgaatattcctataaccgttatgaacacagcatcagaaggtggaccctggggaatggcagtgttagcgatatacgccaaacatcacgcagataaccaaaaccttgcagattttctagacacggaagtgttcaaggatcctgaaagcatgaccttgagcccggaacccgcaggcgtcgcgggataccaggaatttatcaagaattaccaagctgcactgccagtagaggccaaagcgggggatgctataaacgagaaagggtga

SEQ ID NO: 30 - protein, 536 aa, Lactobacillus composti
MNTLEISKAIQAGNVSLGIELGSTQIKSVLVTDDFSTIASGSYVWENKFEQGVWTYPIEEVWNGIQTSYTQMAANVQSKYHENLVHISAIGISAMMHGYLAFDKSDKLLVPFRTWRNNITEEAADKLTDLFDFNIPQRWTIAHLYQSILNQEAHVADLDFVTTLAGYVTWKLSGEKVLGIGDASGVFPIDETTGTYRADLLDKFNQLPEVVQYNWQTTQLLPKVEKAGVQAGTLTAEGAKLLDVNGNLAAGSLMAPPEGDAGTGMVGTNSVRKRTGNISVGTSAFSMVVLDKPLKALHRDIDIVTTPTGAPVAMVHTNNCSSDINAWVAIFKEFAARLGVNLPADQLYQTLFLEATHADPDAGGLLNFSYLSGENITKIKAGRPLFVRTPNSNFNLPNFIQTQLYAAFAPLKIGMDILVKDEHINTEAMIAQGGLFKTPVIGQQVLANALNIPITVMNTASEGGPWGMAVLAIYAKHHADNQNLADFLDTEVFKDPESMTLSPEPAGVAGYQEFIKNYQAALPVEAKAGDAINEKG

SEQ ID NO: 31 - DNA, 1605 bp, Lactobacillus fabifermentans
  CDS (1)..(1605)
atgaacttgatcgagacgagtcaggaaattgagaatgggtccgtatcccttggaatagaattaggctccacaaggataaaagctgttttggtcacttccgatttcaagacaattgcaaccggaagttatgtctgggaaaaccagctggagaacaacatctggacctatgctcttgaagatgtatgggccggtatccagcagagctaccagcaaatggctgcggacgtacaatctaaatatcactgcgaactaacaaaattgaatgcaataggcatcagcgctatgatgcatggatacttagtatttgaccaaagtgggaagcagctagttccgtttaggacgtggcgtaataatatgacagagaccgcagcagatgcactgacggcgctgttcgggtttaacatcccccaaagatggtccatcgctcacttataccaagcaattcttaatgaagagtcccatgtcacggacataaactatctgaccactcttgccggttacgtacactggcagttatcaggtgaaaaggtgttgggcgtgggtgacgctagtggcgtatttccaatagacgaaaccacaggcacctatgacgagacgatgctgacgaaattcaatagcctaacaaacgtacaagcaaacccgtggagaataacagatatcctgcccaagccgctgccagcggggaccgtcgcgggtcatttgacggcagccggagcagccctgctggatccgagcggccagttagcacctggcagtgtgatggccccaccggaaggggatgctgggaccggaatggtggctaccaatagcgtcagaccgaggacggggaatatttctgtcggtacctcagctttctctatgaacgtacttgaccaacccttgtcccaagtctaccgtgacatagatatagtaactacacccgccggggccccggtagcaatggttcatgtcaataactgcagctccgatattaatgcttggagtgggatcttcaacgcatttgcgcagagactgggtgtcaatcttaagccggaccagttgtacgaaactcttttcttggtgtctacgaaagctgatccggacgcgggcaacttggtgaattatagttatcaaagtggagaaaacattacgaaaataaaaacgggccgtcccttgttcgtgaggggtgagaactctaagtttacattaccgaacttcattcagagccaactgtacgctgccttcgcaccgctgaaaatcgggatggatattctggtgaatcaagagaaaatcaagactaatgttatgattgcacaaggtggactattcagaacgccggttattgaacagcaagttttagcaaacgctttgaatatcccgatcacggtgatgtccactgccagcgaagggggaccttggggtatggcagttttggcaatttacgcgagccgtcaccagataggcgataaccttgctgattttctagatagggatgtgttcgttaatccggaaaccatgactttgaatcctgaaccagagggggttgccgggtatcagcagttcattaagaactatcaacaatcccttcaacttgaggcacttgcgggcgaaacggttaaatcctga

SEQ ID NO: 32 - protein, 534 aa, Lactobacillus fabifermentans
MNLIETSQEIENGSVSLGIELGSTRIKAVLVTSDFKTIATGSYVWENQLENNIWTYALEDVWAGIQQSYQQMAADVQSKYHCELTKLNAIGISAMMHGYLVFDQSGKQLVPFRTWRNNMTETAADALTALFGFNIPQRWSIAHLYQAILNEESHVTDINYLTTLAGYVHWQLSGEKVLGVGDASGVFPIDETTGTYDETMLTKFNSLTNVQANPWRITDILPKPLPAGTVAGHLTAAGAALLDPSGQLAPGSVMAPPEGDAGTGMVATNSVRPRTGNISVGTSAFSMNVLDQPLSQVYRDIDIVTTPAGAPVAMVHVNNCSSDINAWSGIFNAFAQRLGVNLKPDQLYETLFLVSTKADPDAGNLVNYSYQSGENITKIKTGRPLFVRGENSKFTLPNFIQSQLYAAFAPLKIGMDILVNQEKIKTNVMIAQGGLFRTPVIEQQVLANALNIPITVMSTASEGGPWGMAVLAIYASRHQIGDNLADFLDRDVFVNPETMTLNPEPEGVAGYQQFIKNYQQSLQLEALAGETVKS

SEQ ID NO: 33 - DNA, 1608 bp, Lactobacillus sakei
  CDS (1)..(1608)
atgaacttggttgagattgcacaagcgattaaatctgggcaggtttcactggggatcgaattgggtagcacccgtataaaagcggtattggtcgcgcaagactttcagaccattgcctctgggtctttcgtttgggagaaccagttggaggacggagtttggacgtacccgatagatgctgtttggaccggcatccaagcctcttacatgcagatggctgcggaggtgcaaagtaaataccacgagccgatcacaacgatcaagagcattggggtttctgccatgatgcatggttatttggcattcagtaaggatgatcaattattagttccttttaggacttggaggaacaacataacggagcaagctgccgataagctaaccgagcaattcaattttaatatccctcagcgttggtctatcgcacatctgtaccaggccattttaaatgatgaggcccatgtatctcaagttgcctttatgacgacactggccggttacgttcattggcagctgagcggagaaaaagtgttagggataggggatgcttccggtgtattcccgatagacccaaagacaggctcctacgatgcacaactagttgcacagttcgatcagatcaaaagagtccaacagcaaccatggcaactagcggacatccttcccgccgtgcttacagcaggacacgcagctggccaattaacatccgccggcgctcaacttttggacccatcaggtaatttgactgcaggatcactaatggcgcctccggagggggatgctggcaccgggatggtagggacgaacagcgtcagaaagaggactggcaacatatccgtcgggacatcagcattcagcatggtcgtcctggaccagcccctgaaacaagtccaccgtgacattgatatggttatgacacctgatggctcacctgtcgccatggtccacacaaataattgtagttccgatattaatgcctgggcgaatttgtttaatgagttcgcagcaagattgggagtatcattggcgccagacagattgtatgagacactattccttgaagcgacccatgctgaccaagacgccgggggactgattaactactcatacctgtcaggggaaaatgtaaccaaaatgcctgccggaaggcccctatttgtcagacagccccatagtcattttaacctgccaaattttattcaaacccagctttacgctgcttttgcacctttgaagatcggaatggatattttagttaaggaggagcagataaaaactgacgtaatgattgcccaaggtggtttgtttaagaccccggtcgttggtcagcaggtccttgctaacgccttaaacacccctataaccgtcatgtcaaatgccggtgaggggggtccctggggcatggcagtactggccatgttcgcagccgacaataatgggcaaactctggatgacttccttgatcacaacgtctttaccaatccagagtcaatgacgctaagccctgaggcggcaggtgtcgccggttaccaaacatttatagagacttaccaggcagggttaccaatcgagtcagccgcgatcgccgcgataaaagatgagaagtga

SEQ ID NO: 34 - protein, 535 aa, Lactobacillus sakei
MNLVEIAQAIKSGQVSLGIELGSTRIKAVLVAQDFQTIASGSFVWENQLEDGVWTYPIDAVWTGIQASYMQMAAEVQSKYHEPITTIKSIGVSAMMHGYLAFSKDDQLLVPFRTWRNNITEQAADKLTEQFNFNIPQRWSIAHLYQAILNDEAHVSQVAFMTTLAGYVHWQLSGEKVLGIGDASGVFPIDPKTGSYDAQLVAQFDQIKRVQQQPWQLADILPAVLTAGHAAGQLTSAGAQLLDPSGNLTAGSLMAPPEGDAGTGMVGTNSVRKRTGNISVGTSAFSMVVLDQPLKQVHRDIDMVMTPDGSPVAMVHTNNCSSDINAWANLFNEFAARLGVSLAPDRLYETLFLEATHADQDAGGLINYSYLSGENVTKMPAGRPLFVRQPHSHFNLPNFIQTQLYAAFAPLKIGMDILVKEEQIKTDVMIAQGGLFKTPVVGQQVLANALNTPITVMSNAGEGGPWGMAVLAMFAADNNGQTLDDFLDHNVFTNPESMTLSPEAAGVAGYQTFIETYQAGLPIESAAIAAIKDEK

SEQ ID NO: 35 - DNA, 1611 bp, Pediococcus lolii
  CDS (1)..(1611)
atggaaatcttgaaaatgaacatcgtagagatctccaaccagatagaacagggtaatgtggctctaggcatagagttgggctccaccagaatcaagaccgtattaataaccaatgattacaagactatagctagcggatcttttgtgtgggagaaccagtacaaggataatatatggacttattcactagaggatgtttggaaaggaattcagacttcttacagcaagatggccgcagaagtgaagagtaagtatcacaaaccactgactaagattaaatctattggcatctctgcgatgatgcatggctacttggcgttcgatcagagcggagacttgctggttccgttcaggacttggcgtaacaatataactgaagagagcgcagctaaactgaccgaagcgttcaactttaacataccgcaaagatggaccattgctcatttgtatcaagctatccttaatggtgaagatcatgtggagaaccttgacttcgtaactaccttggcaggttatgttcactggaagttaagcggggaaaaagtactgggtgttggggacgcttcaggcgtgtttcccatcgacgagcagaccggagactatagtgaaaacatgcttgctacatttgccaacttgcccgaagtaaaaccattctcctgggacatcaaacatgtacttccctctgtaaaaaaagccggcgagaatgccggccaactgtcagcgcagggcgcgaagcttctggatattaccgggaacttgcaagcaggttcacttatggcaccaccggagggtgatgctggaacgggtatggtagggaccaattctgtgagaaaaaggacggggaatatctctgtcggtacctcagcattttctatggtggtattagagaaacccttaaagtccgttcatagagatatcgatgtggtcatgaccccggatggagccgcggttgccatggtccacgtgaataattgcagcagcgatatcaatgcgtgggctcagatattcaacgagttcgcaacgaggctggggataaatttaaccccggataagctatatgaagctctattcttggaagcagctaaagcggatcacgatctgggcggtttggttaattactcatatcaatctggagagaacataacaaatataaaagcaggcagacccctgttcgtaaggaagcctgacagtcactttaatttacccaacttcatgcagacgcagctttattccgcatttgccccgcttaagatagggatggacatcttgtctaaagaagagaatatcaagacagatgttatgattgcccagggaggactttttcgtacagcagtaatagcacagcaggtactttcagacgcgcttaacacccccatcactgtgatgagcaacgccggtgaagggggaccatggggaatggctatccttgcaatgtacgcagcggacaatcaaggtcaatctttaagcgactatttagactataacgtattcaataaccctgagtcaatgaccctttcaccagagccagagggtgtggcggcatataatgaattcacgaagcattacgaggccggtcttccggtggaagctcgtgctggtgaagtgatataa

SEQ ID NO: 36 - protein, 536 aa, Pediococcus lolii
MEILKMNIVEISNQIEQGNVALGIELGSTRIKTVLITNDYKTIASGSFVWENQYKDNIWTYSLEDVWKGIQTSYSKMAAEVKSKYHKPLTKIKSIGISAMMHGYLAFDQSGDLLVPFRTWRNNITEESAAKLTEAFNFNIPQRWTIAHLYQAILNGEDHVENLDFVTTLAGYVHWKLSGEKVLGVGDASGVFPIDEQTGDYSENMLATFANLPEVKPFSWDIKHVLPSVKKAGENAGQLSAQGAKLLDITGNLQAGSLMAPPEGDAGTGMVGTNSVRKRTGNISVGTSAFSMVVLEKPLKSVHRDIDVVMTPDGAAVAMVHVNNCSSDINAWAQIFNEFATRLGINLTPDKLYEALFLEAAKADHDLGGLVNYSYQSGENITNIKAGRPLFVRKPDSHFNLPNFMQTQLYSAFAPLKIGMDILSKEENIKTDVMIAQGGLFRTAVIAQQVLSDALNTPITVMSNAGEGGPWGMAILAMYAADNQGQSLSDYLDYNVFNNPESMTLSPEPEGVAAYNEFTKHYEAGLPVEARAGEVI

SEQ ID NO: 37 - DNA, 1611 bp, Bacillus coagulans
  CDS (1)..(1611)
atgaagaagatgaatcttatagaaacaagccaagcaatacaacaaggaaacgtgtcactggggatagagcttggaagcacgagaattaaggctgtgttggtcaccgatgactttgagactattgcaagcggtgggtatgtgtgggagaacaaattcgagaatggtatctggacgtactcccttgaagaggtgtgggaaggtattcaacgtagttacgcacaaatcgcagccgacgtgcaaggtaaatatcatacaaccctgacgaaaataagcgcgattggggtcagtgccatgatgcacgggtacttggcgttcgcagaaaacggagagttactagttcctttcaggacatggaggaataacattacggagcaagcggccgatgagttgacggagagatttcagtttaatattcctcaaagatggagtatagcacatctttaccaagcgattctgaaccaagaaccccacgtcaaggatatcagatttattactacgctagccggctatgttcattggaaactgtccggcgaaaaagtattgggcatcggcgacgcgagcggggtctttcctgtggatgaagaaactgggacctatagggaggattttttagacactttcgatcaactagaaaacgtggctccgtatccatggcatattagcgagatacttccgaaagtgcttaaagccggcgaatacgcgggcactttaaccccggagggtgccgccctgttggacgttaatggcaatctgcaagcaggaagtttaatggcccctcccgaaggtgacgccggcactggcatggtatgcacgaactccgtcagaaaaagaacaggcaatattagcgttggtacctcagccttctctatgatagtattagatcagccacttgagaaagtgtatagagacatcgatatagttactactccttcaggtgcgcctgtcgctatggtgcacataaataactgctcatctgatataaatgcatgggccaatttattcaagcagtttgctgaaaggttgggcgtcgacctgcctgcggacagactttatgagacgcttttcctagtcgcagcgaaagctgatcccgatgcgggtgggttactaaattacagctacctatcaggcgaaaatattacgaaaatgccagcggggaggcctcttttcgtcaggaagcctgactcagcttttacacttgcgaattttgtccaggcgcaattgtatgcagcttttgcaccactgaagataggtatggatattctgaaaaatgaagaggggataaagacagatgtcttgattgcacaaggggggctattcaagacaccggtcatcggtcaacaggtcttggccaatgcattgaatactccaattactgtgatgagtacagctggtgagggtggtgcgtgggggatggccgtgctagcggtgtacgcaaaaagtggtaatggcaagcaatctctggaggattttctggaccagaacgtgtttacgcatcctgaatctatgactttaagccccgaacccgagggtgtcagtggttatgaagccttcataaagaggtatgaagcaggtctgccggtggaaagtatggctgtcaagagtatagggtag

SEQ ID NO: 38 - protein, 536 aa, Bacillus coagulans
MKKMNLIETSQAIQQGNVSLGIELGSTRIKAVLVTDDFETIASGGYVWENKFENGIWTYSLEEVWEGIQRSYAQIAADVQGKYHTTLTKISAIGVSAMMHGYLAFAENGELLVPFRTWRNNITEQAADELTERFQFNIPQRWSIAHLYQAILNQEPHVKDIRFITTLAGYVHWKLSGEKVLGIGDASGVFPVDEETGTYREDFLDTFDQLENVAPYPWHISEILPKVLKAGEYAGTLTPEGAALLDVNGNLQAGSLMAPPEGDAGTGMVCTNSVRKRTGNISVGTSAFSMIVLDQPLEKVYRDIDIVTTPSGAPVAMVHINNCSSDINAWANLFKQFAERLGVDLPADRLYETLFLVAAKADPDAGGLLNYSYLSGENITKMPAGRPLFVRKPDSAFTLANFVQAQLYAAFAPLKIGMDILKNEEGIKTDVLIAQGGLFKTPVIGQQVLANALNTPITVMSTAGEGGAWGMAVLAVYAKSGNGKQSLEDFLDQNVFTHPESMTLSPEPEGVSGYEAFIKRYEAGLPVESMAVKSIG

SEQ ID NO: 39 - DNA, 729 bp, Lactobacillus plantarum
  CDS (1)..(729)
atgttggaagcattgaagcaagaagtttacgaagctaacatgcaattgccaaagttgggtttagtcacctttacttggggtaacgtatccggtatcgatagagaaaaaggtttgttcgtaattaagccatccggtgttgactacggtgaattgaaacctagtgatttggttgtcgtaaatttgcaaggtgaagttgtcgagggtaaattaaacccatcttcagacacacctacccatactgttttgtacaacgctttcccaaacattggtggtatagttcatacacactctccttgggccgtcgcttacgctgcagcccaaatggatgttccagctatgaatactacacatgcagataccttctatggtgacgttcctgctgcagatgcattgactaaagaagaaatcgaagctgactacgagggtaacacaggtaaaaccatcgttaagacattccaagaaagaggtttggattacgaagccgtcccagcttctttggtatcacaacatggtcctttcgcttggggtccaacccctgcaaaagccgtttataatgcaaaggtcttagaagtagttgccgaagaagactaccacactgcacaattaacaagagcctccagtgaattgccacaatatttgttggataagcattacttgagaaagcacggtgcttctgcatattacggtcaaaataacgcccattcaaaagatcacgctgttagaaagtaa

SEQ ID NO: 40 - protein, 242 aa, Lactobacillus plantarum
MLEALKQEVYEANMQLPKLGLVTFTWGNVSGIDREKGLFVIKPSGVDYGELKPSDLVVVNLQGEVVEGKLNPSSDTPTHTVLYNAFPNIGGIVHTHSPWAVAYAAAQMDVPAMNTTHADTFYGDVPAADALTKEEIEADYEGNTGKTIVKTFQERGLDYEAVPASLVSQHGPFAWGPTPAKAVYNAKVLEVVAEEDYHTAQLTRASSELPQYLLDKHYLRKHGASAYYGQNNAHSKDHAVRK

SEQ ID NO: 41 - DNA, 687 bp, Bacillus licheniformis
  CDS (1)..(687)
atgttggaaagcctaaaggaacaagtgttgaaagccaatctgcagttagaggagtatagactagtgacctttacgtggggtaatgtatcgggtattgacagagaaaacaagcttgttgtcatcaaacctagtggtgttaagtactctgacttgaaagcggaagatttggtggttttgaacatggatggggaaatcgttgaaggtgacttgaaacccagctcagatactccaacacacctttacctgtatagacaattccaatcaattggtggtatagttcatacccactctccatgggcaacatcatgggctcaatctgggaaagatatcccatccttaggaactactcatgcagactattaccatggagacataccatgcactagagaaatggatccagatgagattagatcccaatatgagctgaatacaggcaaagtaattgtcgaaacgttcagacatctagatcctttagctgttcctgctgttttagtgaacaaccatggtcccttttgttggggcaaagatgccttaacagctgtacacaatgccgtagtcttagaagaagtcgctaaaatggcgtataggtcgttaatgcttaatccgtctttgaagaggataaagagtgcattgcaggataaacactactttcgtaagcatggagctgatgcatactatggccaataa

SEQ ID NO: 42 - protein, 228 aa, Bacillus licheniformis
MLESLKEQVLKANLQLEEYRLVTFTWGNVSGIDRENKLVVIKPSGVKYSDLKAEDLVVLNMDGEIVEGDLKPSSDTPTHLYLYRQFQSIGGIVHTHSPWATSWAQSGKDIPSLGTTHADYYHGDIPCTREMDPDEIRSQYELNTGKVIVETFRHLDPLAVPAVLVNNHGPFCWGKDALTAVHNAVVLEEVAKMAYRSLMLNPSLKRIKSALQDKHYFRKHGADAYYGQ

SEQ ID NO: 43 - DNA, 693 bp, Anaerostipes hadrus
  CDS (1)..(693)
atgcttgaacagttgaagaaagaagtttacgaagctaatatggaactcccaaaaagaggtttaatcacctacacgtggggtaatgtttctggtattgatagagaaagcggtctgttcgtaattaagccgtcaggtgttgattatgacaagctgtcacccgaggatatggttgtaatggatttggacggtaacagagttgaaggagaccttaatccgtcttcagacaccccaactcacatagaactttacaaggctttcgaggatttaggtggcatagttcatactcactctccatgggcaacgtcttgggcccaagctggtagatcaatcccctgttacggtacaacccacgccgattacttctatggtgaaatcccatgtgctagatctctgactaaagaagaaatagaagaagcttacgaaaaaaatactggtttagtgatcatcgaaacatttaaggataagaacccagtatacgtcccaggtgttctctgtacaaatcacggtcccttcacgtggggtgctgatgctgctgaagctgttcataacgctgtcgttcttgaggaagttgctaagatggcctacagatgcgaacatctaaatccagaggctaagccagctcctcagtaccttcaagataagcacttctttagaaaacatggtgctaatgcctattatggtcaaggtaaatga

SEQ ID NO: 44 - protein, 230 aa, Anaerostipes hadrus
MLEQLKKEVYEANMELPKRGLITYTWGNVSGIDRESGLFVIKPSGVDYDKLSPEDMVVMDLDGNRVEGDLNPSSDTPTHIELYKAFEDLGGIVHTHSPWATSWAQAGRSIPCYGTTHADYFYGEIPCARSLTKEEIEEAYEKNTGLVIIETFKDKNPVYVPGVLCTNHGPFTWGADAAEAVHNAVVLEEVAKMAYRCEHLNPEAKPAPQYLQDKHFFRKHGANAYYGQGK

SEQ ID NO: 45 - DNA, 699 bp, Bacillus acidiproducens
  CDS (1)..(699)
atgttggaagaattaaagaaagaagtatatgaagctaacatgttactgccagcgtaccatttggttacatttacttggggtaacgtatctggtatagacagggagaaaaatctgtttgtgatcaagccctcgggtgttgactatgatgaactgacaccagagaagatggtagttgttgacctggacggtaacgttgtcgaaggtgatttaaacccatcaagcgatactccaacacattgtgtcctttacaatcattttccaaacatgaagggtatcgtccatacacacagtccatgggctgtttcgttcgctcaagctggactggacatcccggctgctggtactacccatgctgatactttctacggtccggtgccagtgacaagggctatgaaaaaggaggaagttatcactgaatacgaaaagcagaccggtgacgttattgttgaaacgtttgaaaaaaggcaaatagatccggatcaagttccagctgtccttgtcaacgaccacggtccattcacatggggtaagtctgcccatgaagccgttcacaacgctgtcgtgcttgaggaagtggctaaaatgacgtaccatagcctacagctaaacccacaccaaataggtatggatcaatacttgttggataagcacttcctaagaaagcacggaaaaaatgcttactacggtcagacgaagtga

SEQ ID NO: 46 - protein, 232 aa, Bacillus acidiproducens
MLEELKKEVYEANMLLPAYHLVTFTWGNVSGIDREKNLFVIKPSGVDYDELTPEKMVVVDLDGNVVEGDLNPSSDTPTHCVLYNHFPNMKGIVHTHSPWAVSFAQAGLDIPAAGTTHADTFYGPVPVTRAMKKEEVITEYEKQTGDVIVETFEKRQIDPDQVPAVLVNDHGPFTWGKSAHEAVHNAVVLEEVAKMTYHSLQLNPHQIGMDQYLLDKHFLRKHGKNAYYGQTK

SEQ ID NO: 47 - DNA, 699 bp, Fructobacillus pseudoficulneus
  CDS (1)..(699)
atgttgttggaaaagctgaggctggaagtgtatcaagctaacatggctcttccggagttgggtctggtctcatttacgtggggtaacgtatccggatacgacgctcaatctcagctctttgtaataaagccgtctggcctaagttacgaacaacttacgccagccaacctggtcgtgctcgatttgtcaggaaatgtggttgagggtgaactgaagccaagtacagatgctccgactcatgcttacttgtatcaacacttttctggtattggtggtattacacatacgcactcctcatgggcagttgcttatgctgctgctggtctagacgtccccgtagtgtcgaccactcacgctgatacgttctttggtgatgttccgtgtgtaccaactctaacagcgaaggaattgactgaaaattatgaacaaaacactggtaaagctatagtcagaactttcgctgaaagaggtattaatcatttgacgacatccgctgccctgatggctcagcatggtccgttcacctgggggaaagatgctggtgagtctgtctaccacgctaaagtcctcgaagttactgctgagattacacacagagctatgcaacttacccacgctgacatccaccttccacaacatttgttggataagcactaccaaagaaagcacggaaagactgcctactacgggcaagcttga

SEQ ID NO: 48 - protein, 232 aa, Fructobacillus pseudoficulneus
MLLEKLRLEVYQANMALPELGLVSFTWGNVSGYDAQSQLFVIKPSGLSYEQLTPANLVVLDLSGNVVEGELKPSTDAPTHAYLYQHFSGIGGITHTHSSWAVAYAAAGLDVPVVSTTHADTFFGDVPCVPTLTAKELTENYEQNTGKAIVRTFAERGINHLTTSAALMAQHGPFTWGKDAGESVYHAKVLEVTAEITHRAMQLTHADIHLPQHLLDKHYQRKHGKTAYYGQA

SEQ ID NO: 49 - DNA, 696 bp, Listeria ivanovii
  CDS (1)..(696)
atgttagaagccctaaaggaagaagtgtatcaagcgaatgtcgaacttcctaaacaaggtctgataaagtacacatggggtaacgtttctggcattgatcgtgagaaggaattattcgttatcaaaccttctggagttgattacgacacactgcagccggctgatatggtggtctgtgacctagctggtaatgtagtagaaggtaagctgaacccgtcctcggatacacctacccatgctattctttaccagaaataccccgagatcggaggtatcgtgcatacgcactctacctgggctacaatctgggctcaagctggtttggacgtgccggctatgggtacaactcatgctgacactttctacggtacagtgccttgtgctaggttccttactcaagctgaaatcgataggggctacgaaaaggaaacgggtaatgttatcgttgaaacattccaacaaagaggtattgctccaatggctgttccgggtgtgttgttgcatggtcacgggtcatttacatgggggaaagatgctaagtcggccgtgatgaacgctgtagtacttgacgaagtgtgtaaaatgaatctgttcgctagacaactgaaccatttttccgaggaattaccgcaaagaattttggacaaacattatctaaggaagcatggtaaggatgcttactacggtcaaaagtga

SEQ ID NO: 50 - protein, 231 aa, Listeria ivanovii
MLEALKEEVYQANVELPKQGLIKYTWGNVSGIDREKELFVIKPSGVDYDTLQPADMVVCDLAGNVVEGKLNPSSDTPTHAILYQKYPEIGGIVHTHSTWATIWAQAGLDVPAMGTTHADTFYGTVPCARFLTQAEIDRGYEKETGNVIVETFQQRGIAPMAVPGVLLHGHGSFTWGKDAKSAVMNAVVLDEVCKMNLFARQLNHFSEELPQRILDKHYLRKHGKDAYYGQK

SEQ ID NO: 51 - DNA, 699 bp, Megasphaera sp. An286
  CDS (1)..(699)
atgttggaggaactaaagcagcaggtttatgaagctaacatgagattgccgaagcttgaattggttacctttacttgggggaacgtaagtggtattgacagagaaaagggtctatttgttatcaagccatcgggagtcgaatacgaaaagctatcgccagccgacatggtcgtggtagatttggatggtaaagttgtggaaggtgatctgaacccatcatcggatacaatgacgcatgccgtgttatacaaggcttttccgaatattggtgggattgttcatgcccatagcccatgggcggtgtcattcgctcaggctggagtagatttggaagctatgggaacaactcacgctgatactttctacggtgacgtcccggtcacggacgctctgacgaaagaagaaattgaatcagcttatgaagaaaatacgggtaaggtcattgtcagaacgttcgctgagaggggtattgacccagatgctgtaccagcagtgctggtaaagcagcatggtccatttacatggggtccgactgccgcgaaagctgttgaaaccgctaaaatcttagaggtcgtagctgaaatgaactaccatgctttgcagctgacgagggcagatataagagttccgcaatatctgctggacaagcattactatcgtaagcatggtgccaacgcttactacggtcagaaaaaatga

SEQ ID NO: 52 - protein, 232 aa, Megasphaera sp. An286
MLEELKQQVYEANMRLPKLELVTFTWGNVSGIDREKGLFVIKPSGVEYEKLSPADMVVVDLDGKVVEGDLNPSSDTMTHAVLYKAFPNIGGIVHAHSPWAVSFAQAGVDLEAMGTTHADTFYGDVPVTDALTKEEIESAYEENTGKVIVRTFAERGIDPDAVPAVLVKQHGPFTWGPTAAKAVETAKILEVVAEMNYHALQLTRADIRVPQYLLDKHYYRKHGANAYYGQKK

SEQ ID NO: 53 - DNA, 699 bp, Selenomonas sp. oral taxon 149
  CDS (1)..(699)
atgctagaagagttaaagcaagaggtgtatgaagccaatatgatgttgccgaagctgagtatcgtgacgttcacctggggaaacgtttcaggtctggatagagataaaggattatttgttattaagccttcgggggttgaatacgagagacttagacctgaagatatggtggtagttgaccttgacggaaaggtggttgagggggctatgaacccgtcgtctgacactcttacacatatggtgttatatagagaattctcaggtatcaacgctgtagtgcatacccactcgtcatgggctgtttcctttgctcaagctggtgtagagatagatgcgatgggtacgacgcatgctgataatttctatgggacgatcccagttacagaggctttaacgagggaagaaattgaatcaggttatgaagagaataccggttggattatcgttagaacgtttagagaaagggggattgacccagttgctgttccggctgtgctcgttaaacaacatggtccgttcacttggggtgctaccaccaaaaaggctgtagaaaacgctaaggtgcttgatttggtcgctgaaatgaattaccatgcgatgatgttaacgcatgccgacacaagagttccacagtacttactggataagcactatttgcggaagcatggagcgaatgcttactatggccaaaaagcctga

SEQ ID NO: 54 - protein, 232 aa, Selenomonas sp. oral taxon 149
MLEELKQEVYEANMMLPKLSIVTFTWGNVSGLDRDKGLFVIKPSGVEYERLRPEDMVVVDLDGKVVEGAMNPSSDTLTHMVLYREFSGINAVVHTHSSWAVSFAQAGVEIDAMGTTHADNFYGTIPVTEALTREEIESGYEENTGWIIVRTFRERGIDPVAVPAVLVKQHGPFTWGATTKKAVENAKVLDLVAEMNYHAMMLTHADTRVPQYLLDKHYLRKHGANAYYGQKA

SEQ ID NO: 55 - DNA, 1482 bp, Bacillus licheniformis
  CDS (1)..(1482)
atgatccaagccaaaacccatgtgttttggtttgtgacaggatctcaacatctttatggcgaagaagcggttcaagaagttgaagaacattctaagatgatctgcaatggactgaatgatggtgatttaaggttccaagttgagtacaaagctgttgcaacaagcttagatggcgttagaaagttgtttgaggaagctaatagggatgaagaatgtgctggtattatcacgtggatgcatactttctctccggctaaaatgtggatacctgggttgtctgaattgaacaaacccttgttgcactttcatacgcagttcaatagggacatcccttgggataagatcgacatggacttcatgaacataaaccaatcagcccatggggatagagagtacgggttcattggtgctagacttggtattcctcgtaaagtcattgcaggctattgggaagatagagaagtaaagaggtccattgacaagtggatgagtgctgccgttgcatacattgaatcacgtcacataaaagttgcaagatttggagacaacatgagaaacgtggcggttactgaaggagacaaaatagaagcccagattcagttgggttggtccgttgatggttatggcataggtgatctagtgacagaaattaacgccgtaagtgagcaaagtcttagtgaattgatttcggagtatgaggagctgtatgaatggccagaaggagaggcagcaagagaatcagtcaaggaacaagcgagaatcgaattaggcttaaagaggtttctttcatcaggtggatatacagcgtttactaccacatttgaggacctacatggtatgaagcagttacctgggttagcagtacagaggttaatggctgaaggctatggttttggtggtgaaggtgactggaaaacagctgcactggttcgtatgatgaagatgatggctggtggtaaagaaacctccttcatggaagattacacctaccactttgaacctggtaatgagatgatcttaggtagccacatgttagaggtctgtccatctattgccgaacacaaaccaagaattgaagtacatcccttatccatgggtgcaaaagatgatccagcaagattggtctttgatgggatagctggaccagctgtaaatgtgtcattgatagatctaggtggaagattcagattagtcattaacaaggttgaagccgtgaaagtcccgcatgatatgcctaatctgccagttgctagagtactatggaagccacaaccctctttgagaacttcggctgaggcatggattttggctggaggcgctcatcacacttgtctatcgtatcaacttactgcagagcaaatgttggactgggccgaaatgagcggcatagaagccgtcctgattaatcgtgatacgactatcttgaatctaagaaatgagttaaaatggtctgaagcggcttacagattgagaaaattctaa

SEQ ID NO: 56 - protein, 493 aa, Bacillus licheniformis
MIQAKTHVFWFVTGSQHLYGEEAVQEVEEHSKMICNGLNDGDLRFQVEYKAVATSLDGVRKLFEEANRDEECAGIITWMHTFSPAKMWIPGLSELNKPLLHFHTQFNRDIPWDKIDMDFMNINQSAHGDREYGFIGARLGIPRKVIAGYWEDREVKRSIDKWMSAAVAYIESRHIKVARFGDNMRNVAVTEGDKIEAQIQLGWSVDGYGIGDLVTEINAVSEQSLSELISEYEELYEWPEGEAARESVKEQARIELGLKRFLSSGGYTAFTTTFEDLHGMKQLPGLAVQRLMAEGYGFGGEGDWKTAALVRMMKMMAGGKETSFMEDYTYHFEPGNEMILGSHMLEVCPSIAEHKPRIEVHPLSMGAKDDPARLVFDGIAGPAVNVSLIDLGGRFRLVINKVEAVKVPHDMPNLPVARVLWKPQPSLRTSAEAWILAGGAHHTCLSYQLTAEQMLDWAEMSGIEAVLINRDTTILNLRNELKWSEAAYRLRKF

SEQ ID NO: 57 - DNA, 1425 bp, Lactococcus lactis
  CDS (1)..(1425)
atgttggaaaacactcaaaaggaattctggttcgtcacaggttcccagtttctatacggtccagaggcacttgctacagttgagaagaacgccaggaaggtggtcgacgaaatgaatagtagcggtagtttgccctacccgattatttttaagatagtggctaccactgctgaaaacattacacaaattatgaaggaagctaactaccaagacgaagtagcaggtgttatcacatggatgcacacatttagtccagctaagaactggattagaggcacgcagttgctcaataaaccgctccttcatttggctactcaattcttaaatcacattccatacaaaacaattgattttgattacatgaacgtgaaccagtccgctcacggtgacagagaatatgcttttatcaacgctcgtctgaaaaagaataataagattatctttggtcattgggaggacgatgagattagggagcagatcgcgaagtggatgaacgtggccgtcgcctacaacgaatcttataagataaagattgttacttttgccgacaaaatgagggatgtggcagtaacggatggtgacaaggtggaagctcaaattaagttcggttggacggtggattactggggggtcggtgatctcgttgagtacgtgaatgctgttgctgaatctgacattaatcaactatacaaggatctacaaaacaagtatgattacatcgaaggtaacaactctccagagaagtttgaaaagaatgtgaaataccaattgagagaatatttaggtatcaagaagttcatggatgataagggttactcagccttcactacaaatttcgaagacctagtgggtcttgaacaactgccaggtttagccgcccaactacttctggctgatggttacggatttgctggtgaaggtgattggaagactgctgctcttgtcagattattcaaaatcatggcccacaataaagaaacagtgttcatggaagactacactctggatttgaggcagggtcatgaggctatcttggggtcacacatgttagaagtggatccgtctatagctagcgataagccaagagtggaggtacatccactcgacataggtggtaaatccgatccggctagactagtgttcaccggtatgattggggaagcggtggacatcacgatggctgactacggtaacgaattcaagttgatatcttatgaagtgacgggtaacaaaccggaagaggaaaccccgtttttaccagtggctaaacagctgtggactccgaagcaaggtctgaaggctggtgctgagcactggctacgtgctggtggggggcaccacacagtacttagcttcgtagtcgattccgagcagatcggagacttatgcgcaatgtttggattgacgtacatcaaccttggttga

SEQ ID NO: 58 - protein, 474 aa, Lactococcus lactis
MLENTQKEFWFVTGSQFLYGPEALATVEKNARKVVDEMNSSGSLPYPIIFKIVATTAENITQIMKEANYQDEVAGVITWMHTFSPAKNWIRGTQLLNKPLLHLATQFLNHIPYKTIDFDYMNVNQSAHGDREYAFINARLKKNNKIIFGHWEDDEIREQIAKWMNVAVAYNESYKIKIVTFADKMRDVAVTDGDKVEAQIKFGWTVDYWGVGDLVEYVNAVAESDINQLYKDLQNKYDYIEGNNSPEKFEKNVKYQLREYLGIKKFMDDKGYSAFTTNFEDLVGLEQLPGLAAQLLLADGYGFAGEGDWKTAALVRLFKIMAHNKETVFMEDYTLDLRQGHEAILGSHMLEVDPSIASDKPRVEVHPLDIGGKSDPARLVFTGMIGEAVDITMADYGNEFKLISYEVTGNKPEEETPFLPVAKQLWTPKQGLKAGAEHWLRAGGGHHTVLSFVVDSEQIGDLCAMFGLTYINLG

SEQ ID NO: 59 - DNA, 1425 bp, Megasphaera cerevisiae
  CDS (1)..(1425)
atgttgcaggtaaaagaatatgaattttggttcgtagctggttcgcaacacctctacggtgaagaacaactcagaaatgtcgagagagacaccaaggacatggttaagagactcaacgattctggtaccctaccgtacaagattgtgtgtaagggtgttatgaccacagctgactccataacgcaattcatgaaagaagctaactatcaagataaggttgctggtgtgattacatggatgcatactttttccccagctaaaaactggattagggggacgatgttgctgcagaaacctttgcttcatctggccacgcaataccttaataagatcccatacgatacgatcgactttgattatatgaacttgaaccaatcagctcacggtgacagggaatatggatacatcaacgctcgtctgaatctgaagaacaaaatcgtgtatggatactggggtgacgctgatgtccaacaagaaattgctcagtgggaggatgttgctgttgcctataatgagtccttcagaatcaaagtttgcagattcggtgatacaatgagggacgtggctgtgacggaaggggataaggtagaggctcagatcagaatgggttggacggttgattactacccagtggccgatctggttgctgagattaatactgttaacgacgaggatttgaagaaagccttcgctcatctgaggcaggaatacgttcttactcagggtaacaataagctcgaaaagttcgaatgcagtatctatgtacaactcaagcagtacttggggatcagaagattcatggacaagggtggttatacagccttctgtaccaacttccaggacctctgtgggctggaccaactcccaggtcttgctgtccaactgctgatgagggacggctacggttttggggctgagggagactggaagacagctgcccttacgagattactgaaaataatggctcataatgataggacggttttcatggaagattacaccttagatctaagaaaagatcacgaggctattcttggtgctcacatgcttgaagttgatcctaccatcgcctccgatacgccgcgagttgaagtccatccattggatattggtgacaaggaagacccagcccgtcttgtgttcacaggttcagaaggtgacgctgttgatgtcacagtagctgacttcaggcacggcttcagaatgatttcttatccagtaatctgtaggaagccggaagctgaaacgccaaatctgccagtggctaaacaactttggacaccaaaggctggtctcaagaagggcgctgctgcttggatcaaggctggtggtggtcaccatacggtgctttctttcagactcaaggaagaacagatacatgatctggccgtgatgctcgatatggaactagtggaaatttgttga

SEQ ID NO: 60 - protein, 474 aa, Megasphaera cerevisiae
MLQVKEYEFWFVAGSQHLYGEEQLRNVERDTKDMVKRLNDSGTLPYKIVCKGVMTTADSITQFMKEANYQDKVAGVITWMHTFSPAKNWIRGTMLLQKPLLHLATQYLNKIPYDTIDFDYMNLNQSAHGDREYGYINARLNLKNKIVYGYWGDADVQQEIAQWEDVAVAYNESFRIKVCRFGDTMRDVAVTEGDKVEAQIRMGWTVDYYPVADLVAEINTVNDEDLKKAFAHLRQEYVLTQGNNKLEKFECSIYVQLKQYLGIRRFMDKGGYTAFCTNFQDLCGLDQLPGLAVQLLMRDGYGFGAEGDWKTAALTRLLKIMAHNDRTVFMEDYTLDLRKDHEAILGAHMLEVDPTIASDTPRVEVHPLDIGDKEDPARLVFTGSEGDAVDVTVADFRHGFRMISYPVICRKPEAETPNLPVAKQLWTPKAGLKKGAAAWIKAGGGHHTVLSFRLKEEQIHDLAVMLDMELVEIC

SEQ ID NO: 61 - DNA, 1467 bp, Clostridium sp.
  CDS (1)..(1467)
atgctaaagaacaagaagctagaattttggtttgttgtagggagtcaaaatttgtatggtgaggaagctttgaatgctgtcaagaaagacagtaaagaaatagtggactccttaaacgagtctgggaagttgccgtatccgattgtgttcaagacattagctacatcggctgatgagatcaagaatatcgttaaagagattaattatagggacgaagttgctggagttatcacttggatgcataccttttctcctgccaaaatgtggattgccggaacaaaactgttacagaagccattgttgcacttagccactcaattcaacgagaatattccttggaaaaccatagatatggattatatgaacttgcatcagtcagcgcatggtgatagggaatatggcttcataaacgccagattgaataagaacaacaaagtcgtagtcggatactggaaagataatcaagttcagaaagagattgcagaatggatgcaagttgcttacggatacgtagcatcagaaaatattaaggtggccagatttggcgataatatgcgtaatgtggcagttactgaaggagataaggtagaggcacaaattcaattcggctggactgttgactacttcgcgattggtgatttggtagcggaaatgaacaaagtcagccaaaaggacatagatgcaacatacgaggaattcaaggatatttacattctggacataggtgataacgatccagaattttacgaaaatcatgtcaaagaacagatcaagattgaaattggtttaagaaactttctagaagcaggcaactatacggcttttacgaccaatttcgaagatctgtatgggatgaagcaattgcctggcttagctgtccaaagattgaatgcagaaggttatggttttgccggtgaaggcgattggaaaactgcagctcttaatcgtctattcaaaatcatgaccgacaataagaaaactggttttatggaagattacacctatgaactatctgctgggaatgagagaatcttaggtgcccatatgctggaagtagacccaacattagccgcatctaaaccaagagtagtcgtgaagccacttggcataggtgataaagaagctccagctagattgatctttgatggtgtagtcggtgatggtgttgtggtcagcatgcttgatttgggtacacactacagacttctaattaacgaagttaaagctgttaaacccactgaagatgctcctaatttacctgttgcgaaattagtgtggcaaccacaacccaattttaaagacgccgttaaagcatggatctatgctggaggaggacatcacactgttgcaaccttagagttgactgtagaacaggtgtatgattggtccagaatggttggtcttgaaacgatagttatagaccacaacacaaatttgagagacattataaaagagacatcaaggtaa

SEQ ID NO: 62 - protein, 488 aa, Clostridium sp.
MLKNKKLEFWFVVGSQNLYGEEALNAVKKDSKEIVDSLNESGKLPYPIVFKTLATSADEIKNIVKEINYRDEVAGVITWMHTFSPAKMWIAGTKLLQKPLLHLATQFNENIPWKTIDMDYMNLHQSAHGDREYGFINARLNKNNKVVVGYWKDNQVQKEIAEWMQVAYGYVASENIKVARFGDNMRNVAVTEGDKVEAQIQFGWTVDYFAIGDLVAEMNKVSQKDIDATYEEFKDIYILDIGDNDPEFYENHVKEQIKIEIGLRNFLEAGNYTAFTTNFEDLYGMKQLPGLAVQRLNAEGYGFAGEGDWKTAALNRLFKIMTDNKKTGFMEDYTYELSAGNERILGAHMLEVDPTLAASKPRVVVKPLGIGDKEAPARLIFDGVVGDGVVVSMLDLGTHYRLLINEVKAVKPTEDAPNLPVAKLVWQPQPNFKDAVKAWIYAGGGHHTVATLELTVEQVYDWSRMVGLETIVIDHNTNLRDIIKETSR

SEQ ID NO: 63 - DNA, 1467 bp, Clostridium acetobutylicum
  CDS (1)..(1467)
atgttggaaaacaagaagatggagttttggtttgttgtcggaagtcaacacttgtatggagaagaagcgctgaaagaagtcaggaagaattcggaaactatagtggatgaacttaacaaaagtgcaaatttaccttacaaaatcattttcaaggacttagcgacttcagccgacaagataaaggaaataatgaaagaagttaactatagagatgaagttgctggcgtgattacgtggatgcatacgttttcaccagctaaaatgtggattgcaggaaccaaaatactgcaaaagccattgttacactttgccacgcaatacaatgaaaatattccctggaaaacaatcgatatggattacatgaatctacatcaatcagcacatggggacagagaatatgggttcatcaacgccagactaaagaaacacaacaaagtcgttgtaggctactggaaggacaaagaagttcagaaacaggtgtctgattggatgaaggtagctgctggttatatcgcttctgagtcgatcaaagtagcgagatttggggataatatgaggaatgtagcagtaactgaaggtgataaagtagaggctcaaatccaattcggttggacagttgactacttcggcattggtgatttagtcgcagaaatggataaggtttcccaagatgaaattgacaagacatacgaagaatttaaggacttatacattttggatccgggtgaaaacgatcctgccttctacgaaaagcaagtgaaagaacagatcaaaatagagattggacttagacgtttccttgagaaggggaattataacgcttttactacaaactttgaggatctttatggcatgaaacaattacctggtttagcagttcaaagattgaatgccgaaggctatggttttgctggtgaaggcgattggaaaacagctgcgttagatagactattgaaagtaatgaccaacaataccgcaacaggttttatggaggattacacctatgagttgtctcgtggtaatgagaaagcattgggtgctcatatgctggaagtggatcccacttttgcatccgacaagccaaaagtgattgttaagccactaggtattggtgacaaagaggatcctgctagattgattttcaatggatctactggtaaaggtgttgctgtgagcatgttggatttgggaacacactataggcttatcattaacggattgactgccgttaaacctgacgaggatatgccaaatctgccagtcgccaaaatggtctggaagccagaaccgaatttcatagagggcgtcaaatcctggatttatgcaggaggtggtcatcataccgttgtcagcttagaattgactgttgaacaggtttatgattggagtagaatggttggtttagaaacggtaataattgacaaggatactaaactaagagacataatagaaaagaccacaaaataa

SEQ ID NO: 64 - protein, 488 aa, Clostridium acetobutylicum
MLENKKMEFWFVVGSQHLYGEEALKEVRKNSETIVDELNKSANLPYKIIFKDLATSADKIKEIMKEVNYRDEVAGVITWMHTFSPAKMWIAGTKILQKPLLHFATQYNENIPWKTIDMDYMNLHQSAHGDREYGFINARLKKHNKVVVGYWKDKEVQKQVSDWMKVAAGYIASESIKVARFGDNMRNVAVTEGDKVEAQIQFGWTVDYFGIGDLVAEMDKVSQDEIDKTYEEFKDLYILDPGENDPAFYEKQVKEQIKIEIGLRRFLEKGNYNAFTTNFEDLYGMKQLPGLAVQRLNAEGYGFAGEGDWKTAALDRLLKVMTNNTATGFMEDYTYELSRGNEKALGAHMLEVDPTFASDKPKVIVKPLGIGDKEDPARLIFNGSTGKGVAVSMLDLGTHYRLIINGLTAVKPDEDMPNLPVAKMVWKPEPNFIEGVKSWIYAGGGHHTVVSLELTVEQVYDWSRMVGLETVIIDKDTKLRDIIEKTTK

SEQ ID NO: 65 - DNA, 1494 bp, Bacillus akibai
  CDS (1)..(1494)
atgttaacaataaagaagtatcaattctggtttgttacaggttcgcaacacttgtacgggccggaaacgattgaagaagtgcaggatcattctaggcagattgttcaagctctgaacgaagacgaatcaattccgttcgaaattgtattgaagccagtgctgaccacggcggatgccattagaaggctttgtatggatgctaacactgatgaaaactgcgccggtataatgacatggatgcataccttctccccggctaaaatgtggattgccggtctttctgaactgagaaaaccgttattgcatctacatacacagtttaacagagaaattccatgggactctatcgatatggactttatgaatactaaccaaagcgcccatggagatagggaatacggtttcattggtgctagaatgaacatcgctagaaaggtggtggtaggtcactggcaagacaaacaagttagggaaagaacagctaattggatgagaacagctacagcggtaacggacggtactaatttaaaagtggctagatttggtgataacatgagaaatgttgctgtgactgaaggtgataaggtcgaagctcaaattaaactaggttggacaaccgatgcttacggtattggtgacctcgtccagagaatgaacgacgtcacggaccaacaagtaaaggctctgtacgaagaatacaacgagctttacgatatcgacccgcaggcaaattcaagcgaagcttacagagactcaatcctataccaagccagaatcgaacttggtctgaaggcctttttggaagagggtggttactcagccttcaccacaaacttcgaggatcttcacggtatgaaacagcttccaggtctcgcctcgcaaagattgatggctcaaggttacggtttcggtggtgaaggagattggaagacggctgctctaacgagaatgatgaagatcattgctgacggtaagggtacgagttttatggaagattacacttatcactttgaagaaggtaatgaattggttttggggtcgcatatgttggagatttgtccaactatttccgcgacaaagccaaagattgaagtgcatccattagggatcggtggtaaggaggatccggctaggttggtttttaatggtgctgaaggtagggccttgaacgcctcgattattgatatcggtaatagattcagattggtagttaatgaagttgacgccgtcaagccagtgaaggagatgccaaagctaccggtggctcaagtactgtggaagccacagccgagtctgtcagaatctgccgaagcttggatacacgctggtggcgcacaccacacggtgttctctaccgtcgttacaccagaacagctgtacgattgggccacgatgactgacatcgagtgcgtgtttatcaataattctactaacgtcatgcaattccaaaacgaattgagatggaatgacatcgctaggaaattcgatagatga

SEQ ID NO: 66 - protein, 497 aa, Bacillus akibai
MLTIKKYQFWFVTGSQHLYGPETIEEVQDHSRQIVQALNEDESIPFEIVLKPVLTTADAIRRLCMDANTDENCAGIMTWMHTFSPAKMWIAGLSELRKPLLHLHTQFNREIPWDSIDMDFMNTNQSAHGDREYGFIGARMNIARKVVVGHWQDKQVRERTANWMRTATAVTDGTNLKVARFGDNMRNVAVTEGDKVEAQIKLGWTTDAYGIGDLVQRMNDVTDQQVKALYEEYNELYDIDPQANSSEAYRDSILYQARIELGLKAFLEEGGYSAFTTNFEDLHGMKQLPGLASQRLMAQGYGFGGEGDWKTAALTRMMKIIADGKGTSFMEDYTYHFEEGNELVLGSHMLEICPTISATKPKIEVHPLGIGGKEDPARLVFNGAEGRALNASIIDIGNRFRLVVNEVDAVKPVKEMPKLPVAQVLWKPQPSLSESAEAWIHAGGAHHTVFSTVVTPEQLYDWATMTDIECVFINNSTNVMQFQNELRWNDIARKFDR

SEQ ID NO: 67 - DNA, 1494 bp, Bacillus halodurans
  CDS (1)..(1494)
atgttgcaaacgaaaccgtacacgttttggttcattacaggttcgcagcatctttacggtgaagacgctatcgaacaagtcaggcaacattcgcagactatggtagagaagcttaacaaaattggtgaacttccgtacacaatagagttaaaggaagtcttaacgacaccagatgccatcaggaaaatggtgattgctgctaattctgatgatgattgtgctggtatgattacgtggatgcatacgttttcacctgctaagatgtggattaacggtcttaagcagctgaaaaagcctctgttacatctacacacgcaattcaatagggaaattccatacgatgatatagacatggacttcatgaacttaaaccagtcggctcatggtgacagagaatacggtcacattggtgccagattgaatatctctagaaaggttatcgtgggtcactggcaaaataacgatgtccaggaaagattgggtgcttggatgagaacggctgccgcgtttgtggacggtcaccatctaaaggtcgctagattcggtgataacatgagggaggtcgccgtcactgagggtgataaagttgaagctcaaattcaatttggttggtcaattacagcttttggtattggtgatctggttgaaaagatgaaggctgtatctgaggacgaagttaggaggcttttcgacgaatatcaagagctgtatagactttcaccgtcaattttggaacaagacgaagtaaaggccgctgtactggaacaggccaagatggagctggctctcaaggaattccttgaagaagggggttatacagcttttactactaactttgaagatcttcatgggatgaaacaactaccgggtttagctgttcaaagattaatggctgaaggttacggttttggcggtgaaggtgactggaaaacggcggccctattaaggatgatgaaaataatcgccgacggtaagggaacttcatttatggaagattatacatatcacctggctgaaggcaacgaacttgtccttggttcacatatgctggaaatttgcccaacaatcgccgctaatcagccagaaattcaagttcacccattggggattgggggtaaggaagatcctgctagattggtgtttgatggtgctgatggtccagccctaaacgcttccctgattgatctcggtcataggtttaggttggttgtgaatgaagttgaggctataaaaccagaaagggacatgcccaagcttcctgtagctaaggttctctggaagtgcaaaccgtctctatccgaagctactgaggcttggatacatgctggtggggctcatcacactgttttttcctttgaagtcacgccagaacagttatatgattgggctacgctggcggacattgaagtggtctttattaatgacaaaaccgacgtcttgcaattccaacaacagttgcaatggaacgaagccttcagaaggttattcaagtga

SEQ ID NO: 68 - protein, 497 aa, Bacillus halodurans
MLQTKPYTFWFITGSQHLYGEDAIEQVRQHSQTMVEKLNKIGELPYTIELKEVLTTPDAIRKMVIAANSDDDCAGMITWMHTFSPAKMWINGLKQLKKPLLHLHTQFNREIPYDDIDMDFMNLNQSAHGDREYGHIGARLNISRKVIVGHWQNNDVQERLGAWMRTAAAFVDGHHLKVARFGDNMREVAVTEGDKVEAQIQFGWSITAFGIGDLVEKMKAVSEDEVRRLFDEYQELYRLSPSILEQDEVKAAVLEQAKMELALKEFLEEGGYTAFTTNFEDLHGMKQLPGLAVQRLMAEGYGFGGEGDWKTAALLRMMKIIADGKGTSFMEDYTYHLAEGNELVLGSHMLEICPTIAANQPEIQVHPLGIGGKEDPARLVFDGADGPALNASLIDLGHRFRLVVNEVEAIKPERDMPKLPVAKVLWKCKPSLSEATEAWIHAGGAHHTVFSFEVTPEQLYDWATLADIEVVFINDKTDVLQFQQQLQWNEAFRRLFK

SEQ ID NO: 69 - DNA, 1374 bp, Oenococcus oeni
  CDS (1)..(1374)
atgcttaagacaaatgactacaagttttggttcgtaacaggtagccaattcctatacggtccagaacagctgcagcacgtggaagatgacgctagagacatagtagagaagctgaacaacagtggtaagctgccatacccaatcgaattcaagttagttgctacgacagctgataacataacgaagtttatgaaggacgcaaactacgatgattctgttgcgggtgtaataacgtggatgcatacattctcaccagctaagaactggattagggggaccgaattgctgcaaaagccattgttacatcttgccacacaatacttggatcacatcccatatgatacaattgatttcgattacatgaacttgaatcaatcggcccatggggatagggaatacgcttttatcaatgctaggctaagactgcacaacaagataattttcgggcactgggctgacgaaggtattcaaaaacaaatcagcaaatggatggatactgctgttgcctacaatgaatcttacaagattaaggttgtgacttttgctgataaaatgagaaacgttgctgtgactgacggtgacaaaattgaggctcaaattcaattaggttggactgtcgattactggggtgttggtgatttggttgaatatgtgaacgctgtcgatgaaacgaagatcgacaagttgtacgccgaactgcaggataaatacaactttatttcgggtgacaatagtccggagaagttcgaacataacgtgaaataccaaattagagaatacctagcaattaagaagttcatggatgaaaagggatactcggcttttacgacaaactttgaagatctggtgggtctcgaacaattgcctggtctggcggtacaaatgttgatggctgagggttacggtttcgctggagagggtgattggaaaacagccgccctggatagactattgaagatattgagtcataacgaagcgactgctttcatggaggattacacattagacttcagatcaggacaccaggctatcctcggtagtcatatgcttgaagtggacccaaccatagcttctgataagcccagggtcgaagttcatccgctggacatcggtggtaaggacgaccctgcgagactggtgttcactggtagatcaggtgatgccgttgatgttacactggccgattttggagatgaatttaagttgatttcatacgatgtgacgggtaataaaccggagaaagaaactccgcaccttcccgtcgctaagcagttgtggactccaaaggtcggtttgaagaacggtgctgaagcttggctaacggctggagggggtcatcacactgttttgactttcgctgttgattccgagtga

SEQ ID NO: 70 - protein, 457 aa, Oenococcus oeni
MLKTNDYKFWFVTGSQFLYGPEQLQHVEDDARDIVEKLNNSGKLPYPIEFKLVATTADNITKFMKDANYDDSVAGVITWMHTFSPAKNWIRGTELLQKPLLHLATQYLDHIPYDTIDFDYMNLNQSAHGDREYAFINARLRLHNKIIFGHWADEGIQKQISKWMDTAVAYNESYKIKVVTFADKMRNVAVTDGDKIEAQIQLGWTVDYWGVGDLVEYVNAVDETKIDKLYAELQDKYNFISGDNSPEKFEHNVKYQIREYLAIKKFMDEKGYSAFTTNFEDLVGLEQLPGLAVQMLMAEGYGFAGEGDWKTAALDRLLKILSHNEATAFMEDYTLDFRSGHQAILGSHMLEVDPTIASDKPRVEVHPLDIGGKDDPARLVFTGRSGDAVDVTLADFGDEFKLISYDVTGNKPEKETPHLPVAKQLWTPKVGLKNGAEAWLTAGGGHHTVLTFAVDSE

SEQ ID NO: 71 - DNA, 39 bp, Artificial
  Synthetic DNA
ctggaattcgcccttttgaggttataggggcttagcatc

SEQ ID NO: 72 - DNA, 40 bp, Artificial
  Synthetic DNA
gacccgtggctgcgagtcagtttttccagaaacctccatg

SEQ ID NO: 73 - DNA, 18 bp, Artificial
  Synthetic DNA
tcgcagccacgggtcaac

SEQ ID NO: 74 - DNA, 37 bp, Artificial
  Synthetic DNA
ttttattattagtcttttttttttttgacaatatctg

SEQ ID NO: 75 - DNA, 24 bp, Artificial
  Synthetic DNA
atggcagttgaggagaacaatatg

SEQ ID NO: 76 - DNA, 24 bp, Artificial
  Synthetic DNA
ttattctagcatggccttgtacca

SEQ ID NO: 77 - DNA, 43 bp, Artificial
  Synthetic DNA
gccatgctagaataataaagtaagagcgctacattggtctacc

SEQ ID NO: 78 - DNA, 24 bp, Artificial
  Synthetic DNA
ttactccgcaacgcttttctgaac

SEQ ID NO: 79 - DNA, 25 bp, Artificial
  Synthetic DNA
tgacggtatcgataagcttgatatc

SEQ ID NO: 80 - DNA, 64 bp, Artificial
  Synthetic DNA
ataacttcgtatagcatacattatacgaagttatacgacatcgtcgaatatgattcagggtaac

SEQ ID NO: 81 - DNA, 22 bp, Artificial
  Synthetic DNA
acgacatcgtcgaatatgattc

SEQ ID NO: 82 - DNA, 33 bp, Artificial
  Synthetic DNA
tattaatttagtgtgtgtatttgtgtttgtgtg

SEQ ID NO: 83 - DNA, 36 bp, Artificial
  Synthetic DNA
cacactaaattaataatgaaaaagcctgaactcacc

SEQ ID NO: 84 - DNA, 33 bp, Artificial
  Synthetic DNA
tttagtagacatgcactattcctttgccctcgg

SEQ ID NO: 85 - DNA, 37 bp, Artificial
  Synthetic DNA
tgcatgtctactaaactcacaaattagagcttcaatt

SEQ ID NO: 86 - DNA, 26 bp, Artificial
  Synthetic DNA
gggtaataactgatataattaaattg

SEQ ID NO: 87 - DNA, 37 bp, Artificial
  Synthetic DNA
attatacgaagttattgacaccgattatttaaagctg

SEQ ID NO: 88 - DNA, 54 bp, Artificial
  Synthetic DNA
ataatgtatgctatacgaagttatgggtaataactgatataattaaattgaagc

SEQ ID NO: 89 - DNA, 43 bp, Artificial
  Synthetic DNA
aaagctgcagcatacgaggaatgtgattataaatccctttatg

SEQ ID NO: 90 - DNA, 46 bp, Artificial
  Synthetic DNA
gcagaattcgcccttcatatacgttagtgaaaagaaaagctttttg

SEQ ID NO: 91 - DNA, 23 bp, Artificial
  Synthetic DNA
aagggcgaattctgcagatatcc

SEQ ID NO: 92 - DNA, 22 bp, Artificial
  Synthetic DNA
aagggcgaattccagcacactg

SEQ ID NO: 93 - DNA, 44 bp, Artificial
  Synthetic DNA
acggccagtgaattcggctgatgtaatggtattgttattcaacc

SEQ ID NO: 94 - DNA, 19 bp, Artificial
  Synthetic DNA
atttaccagcatcagcgcc

SEQ ID NO: 95 - DNA, 38 bp, Artificial
  Synthetic DNA
ctgatgctggtaaattagcgttgaatgttagcgtcaac

SEQ ID NO: 96 - DNA, 20 bp, Artificial
  Synthetic DNA
tttgtttgtttatgtgtgtt

SEQ ID NO: 97 - DNA, 35 bp, Artificial
  Synthetic DNA
acataaacaaacaaaatgaatttggtcgaaaccgc

SEQ ID NO: 98 - DNA, 42 bp, Artificial
  Synthetic DNA
agcgctcttactttattagtatttaatagcttgaccagcggc

SEQ ID NO: 99 - DNA, 27 bp, Artificial
  Synthetic DNA
atgaacactctggagatatcaaaggcg

SEQ ID NO: 100 - DNA, 26 bp, Artificial
  Synthetic DNA
tcaccctttctcgtttatagcatccc

SEQ ID NO: 101 - DNA, 25 bp, Artificial
  Synthetic DNA
atgaacttgatcgagacgagtcagg

SEQ ID NO: 102 - DNA, 24 bp, Artificial
  Synthetic DNA
tcaggatttaaccgtttcgcccgc

SEQ ID NO: 103 - DNA, 26 bp, Artificial
  Synthetic DNA
atgaacttggttgagattgcacaagc

SEQ ID NO: 104 - DNA, 25 bp, Artificial
  Synthetic DNA
tcacttctcatcttttatcgcggcg

SEQ ID NO: 105 - DNA, 25 bp, Artificial
  Synthetic DNA
atggaaatcttgaaaatgaacatcg

SEQ ID NO: 106 - DNA, 24 bp, Artificial
  Synthetic DNA
ttatatcacttcaccagcacgagc

SEQ ID NO: 107 - DNA, 23 bp, Artificial
  Synthetic DNA
atgggtttgatgaatgtcgctgc

SEQ ID NO: 108 - DNA, 24 bp, Artificial
  Synthetic DNA
ctactctgctaatgccgtaccagc

SEQ ID NO: 109 - DNA, 17 bp, Artificial
  Synthetic DNA
atggacatgaccgccgc

SEQ ID NO: 110 - DNA, 37 bp, Artificial
  Synthetic DNA
agcgctcttactttactagccgttgtaagtcaacgcg

SEQ ID NO: 111 - DNA, 24 bp, Artificial
  Synthetic DNA
atggatcagaacatccgtcaagcg

SEQ ID NO: 112 - DNA, 27 bp, Artificial
  Synthetic DNA
ctaccccctcccgttttctattaaatg

SEQ ID NO: 113 - DNA, 21 bp, Artificial
  Synthetic DNA
atgggtaacgtaaaggaaacg

SEQ ID NO: 114 - DNA, 23 bp, Artificial
  Synthetic DNA
ttaaaccaggttcttcaaagcgc

SEQ ID NO: 115 - DNA, 28 bp, Artificial
  Synthetic DNA
taaagtaagagcgctacattggtctacc

SEQ ID NO: 116 - DNA, 24 bp, Artificial
  Synthetic DNA
ttactccgcaacgcttttctgaac

SEQ ID NO: 117 - DNA, 64 bp, Artificial
  Synthetic DNA
tataatgtatgctatacgaagttatagcttgcaaattaaagccttcgagcgtcccaaaaccttc

SEQ ID NO: 118 - DNA, 49 bp, Artificial
  Synthetic DNA
atagcatacattatacgaacggtatgacaccgattatttaaagctgcag

SEQ ID NO: 119 - DNA, 22 bp, Artificial
  Synthetic DNA
agcttgcaaattaaagccttcg

SEQ ID NO: 120 - DNA, 26 bp, Artificial
  Synthetic DNA
ttagttatgtcacgcttacattcacg

SEQ ID NO: 121 - DNA, 38 bp, Artificial
  Synthetic DNA
gcgtgacataactaatcaatcaccatcttccaacaatc

SEQ ID NO: 122 - DNA, 37 bp, Artificial
  Synthetic DNA
caaggagaaaaaaccatgtctaacttgttgactgttc

SEQ ID NO: 123 - DNA, 29 bp, Artificial
  Synthetic DNA
ggttttttctccttgacgttaaagtatag

SEQ ID NO: 124 - DNA, 76 bp, Artificial
  Synthetic DNA
tgcatgtctactaaactcacaaattagagcttcaatttaattatatcagttattacccacggattagaagccgccg

SEQ ID NO: 125 - DNA, 26 bp, Artificial
  Synthetic DNA
gggtaataactgatataattaaattg

SEQ ID NO: 126 - DNA, 29 bp, Artificial
  Synthetic DNA
tgcatgtctactaaactcacaaattagag

SEQ ID NO: 127 - DNA, 23 bp, Artificial
  Synthetic DNA
ttagccctcccacacataaccag

SEQ ID NO: 128 - DNA, 23 bp, Artificial
  Synthetic DNA
catggccaagcctttgtctcaag

SEQ ID NO: 129 - DNA, 35 bp, Artificial
  Synthetic DNA
cccttagattagattgctatgctttctttctaatg

SEQ ID NO: 130 - DNA, 48 bp, Artificial
  Synthetic DNA
atagcatacattatacgaagttatcccacacaccatagcttcaaaatg

SEQ ID NO: 131 - DNA, 26 bp, Artificial
  Synthetic DNA
tacgaacggtaagggaaagatatgag

SEQ ID NO: 132 - DNA, 48 bp, Artificial; identical to SEQ ID NO: 130
  Synthetic DNA

SEQ ID NO: 133 - DNA, 32 bp, Artificial
  Synthetic DNA
tctagttggttcttgacatttttcaaataatc

SEQ ID NO: 134 - DNA, 38 bp, Artificial
  Synthetic DNA
tccccgggtaccgagtattccttgttttgttcagcctg

SEQ ID NO: 135 - DNA, 22 bp, Artificial
  Synthetic DNA
acccggggatcctctagagtcg

SEQ ID NO: 136 - DNA, 23 bp, Artificial
  Synthetic DNA
gaattcactggccgtcgttttac

SEQ ID NO: 137 - DNA, 47 bp, Artificial
  Synthetic DNA
ctggaattcgcccttgtatgaccacattctatactgagaagagtgcc

SEQ ID NO: 138 - DNA, 44 bp, Artificial
  Synthetic DNA
taacattcaacgctatattggaatgaggaaatttcggtaaaaac

SEQ ID NO: 139 - DNA, 26 bp, Artificial
  Synthetic DNA
tagcgttgaatgttagcgtcaacaac

SEQ ID NO: 140 - DNA, 30 bp, Artificial
  Synthetic DNA
tttgtttgtttatgtgtgtttattcgaaac

SEQ ID NO: 141 - DNA, 37 bp, Artificial
  Synthetic DNA
acataaacaaacaaaatgttggaagcattgaagcaag

SEQ ID NO: 142 - DNA, 45 bp, Artificial
  Synthetic DNA
agcgctcttactttattactttctaacagcgtgatcttttgaatg

SEQ ID NO: 143 - DNA, 40 bp, Artificial
  Synthetic DNA
acataaacaaacaaaatgttggagcagttaaaggaagaag

SEQ ID NO: 144 - DNA, 44 bp, Artificial
  Synthetic DNA
agcgctcttactttattatttctgaccgtaataggcattcttac

SEQ ID NO: 145 - DNA, 40 bp, Artificial
  Synthetic DNA
acataaacaaacaaaatgttggaaagcctaaaggaacaag

SEQ ID NO: 146 - DNA, 41 bp, Artificial
  Synthetic DNA
agcgctcttactttattattggccatagtatgcatcagctc

SEQ ID NO: 147 - DNA, 25 bp, Artificial
  Synthetic DNA
atgcttgaacagttgaagaaagaag

SEQ ID NO: 148 - DNA, 24 bp, Artificial
  Synthetic DNA
tcatttaccttgaccataataggc

SEQ ID NO: 149 - DNA, 22 bp, Artificial
  Synthetic DNA
atgctagaaaagttaaagcagg

SEQ ID NO: 150 - DNA, 24 bp, Artificial
  Synthetic DNA
tcattgaccgtagtaagcattctc

SEQ ID NO: 151 - DNA, 22 bp, Artificial
  Synthetic DNA
atgttggaagaattaaagaaag

SEQ ID NO: 152 - DNA, 24 bp, Artificial
  Synthetic DNA
tcacttcgtctgaccgtagtaagc

SEQ ID NO: 153 - DNA, 25 bp, Artificial
  Synthetic DNA
atgctggaacaacttaaggaggaag

SEQ ID NO: 154 - DNA, 25 bp, Artificial
  Synthetic DNA
tcagtgcttatttttttgaccgtag

SEQ ID NO: 155 - DNA, 25 bp, Artificial
  Synthetic DNA
atgttgttggaaaagctgaggctgg

SEQ ID NO: 156 - DNA, 21 bp, Artificial
  Synthetic DNA
tcaagcttgcccgtagtaggc

SEQ ID NO: 157 - DNA, 22 bp, Artificial
  Synthetic DNA
atgttagaagccctaaaggaag

SEQ ID NO: 158 - DNA, 24 bp, Artificial
  Synthetic DNA
tcacttttgaccgtagtaagcatc

SEQ ID NO: 159 - DNA, 25 bp, Artificial
  Synthetic DNA
atgttggaggaactaaagcagcagg

SEQ ID NO: 160 - DNA, 24 bp, Artificial
  Synthetic DNA
tcattttttctgaccgtagtaagc

SEQ ID NO: 161 - DNA, 25 bp, Artificial
  Synthetic DNA
atgctagaagagttaaagcaagagg

SEQ ID NO: 162 - DNA, 24 bp, Artificial
  Synthetic DNA
tcaggctttttggccatagtaagc

SEQ ID NO: 163 - DNA, 43 bp, Artificial; identical to SEQ ID NO: 77
  Synthetic DNA

SEQ ID NO: 164 - DNA, 24 bp, Artificial
  Synthetic DNA
ttactccgcaacgcttttctgaac

SEQ ID NO: 165 - DNA, 25 bp, Artificial
  Synthetic DNA
tgacggtatcgataagcttgatatc

SEQ ID NO: 166 - DNA, 64 bp, Artificial; identical to SEQ ID NO: 80
  Synthetic DNA

SEQ ID NO: 167 - DNA, 22 bp, Artificial
  Synthetic DNA
acgacatcgtcgaatatgattc

SEQ ID NO: 168 - DNA, 33 bp, Artificial; identical to SEQ ID NO: 82
  Synthetic DNA

SEQ ID NO: 169 - DNA, 23 bp, Artificial
  Synthetic DNA
atgagccatattcaacgggaaac

SEQ ID NO: 170 - DNA, 38 bp, Artificial
  Synthetic DNA
tttagtagacatgcattacaaccaattaaccaattctg

SEQ ID NO: 171 - DNA, 37 bp, Artificial; identical to SEQ ID NO: 85
  Synthetic DNA

SEQ ID NO: 172 - DNA, 26 bp, Artificial
  Synthetic DNA
gggtaataactgatataattaaattg

SEQ ID NO: 173 - DNA, 37 bp, Artificial; identical to SEQ ID NO: 87
  Synthetic DNA

SEQ ID NO: 174 - DNA, 54 bp, Artificial; identical to SEQ ID NO: 88
  Synthetic DNA

SEQ ID NO: 175 - DNA, 40 bp, Artificial
  Synthetic DNA
aaagctgcagcatacatgaaatgatgcatataagtagcgc

SEQ ID NO: 176 - DNA, 42 bp, Artificial
  Synthetic DNA
gcagaattcgcccttagtgtttgcttaatttacataggaccc

SEQ ID NO: 177 - DNA, 23 bp, Artificial
  Synthetic DNA
aagggcgaattctgcagatatcc

SEQ ID NO: 178 - DNA, 22 bp, Artificial
  Synthetic DNA
aagggcgaattccagcacactg

SEQ ID NO: 179 - DNA, 27 bp, Artificial
  Synthetic DNA
ctaccctattttctcttaccagcgaac

SEQ ID NO: 180 - DNA, 34 bp, Artificial
  Synthetic DNA
gacccgtggctgcgattttggccaaatgccacag

SEQ ID NO: 181 - DNA, 18 bp, Artificial
  Synthetic DNA
tcgcagccacgggtcaac

SEQ ID NO: 182 - DNA, 37 bp, Artificial; identical to SEQ ID NO: 74
  Synthetic DNA

SEQ ID NO: 183 - DNA, 44 bp, Artificial
  Synthetic DNA
agactaataataaaaatgttgtccgttccagattatgaattttg

SEQ ID NO: 184 - DNA, 45 bp, Artificial
  Synthetic DNA
ttgctctcaatccgcttattttaagaaagcctttgtcataccaac

SEQ ID NO: 185 - DNA, 39 bp, Artificial
  Synthetic DNA
agactaataataaaaatgttgaccactggtaagaaggag

SEQ ID NO: 186 - DNA, 42 bp, Artificial
  Synthetic DNA
ttgctctcaatccgcttatttgatcacgacgtattcaagatc

SEQ ID NO: 187 - DNA, 37 bp, Artificial
  Synthetic DNA
agactaataataaaaatgatccaagccaaaacccatg

SEQ ID NO: 188 - DNA, 42 bp, Artificial
  Synthetic DNA
ttgctctcaatccgcttagaattttctcaatctgtaagccgc

SEQ ID NO: 189 - DNA, 44 bp, Artificial
  Synthetic DNA
agactaataataaaaatgctaaagaacaagaagctagaattttg

SEQ ID NO: 190 - DNA, 48 bp, Artificial
  Synthetic DNA
ttgctctcaatccgcttaccttgatgtctcttttataatgtctctcaa

SEQ ID NO: 191 - DNA, 39 bp, Artificial
  Synthetic DNA
agactaataataaaaatgttggaaaacaagaagatggag

SEQ ID NO: 192 - DNA, 53 bp, Artificial
  Synthetic DNA
ttgctctcaatccgcttattttgtggtcttttctattatgtctcttagtttag

SEQ ID NO: 193 - DNA, 44 bp, Artificial
  Synthetic DNA
agactaataataaaaatgttggaagtcaagaattacgaattctg

SEQ ID NO: 194 - DNA, 39 bp, Artificial
  Synthetic DNA
ttgctctcaatccgctcagcagatttcaaccaagttgac

SEQ ID NO: 195 - DNA, 30 bp, Artificial
  Synthetic DNA
atgttaacaataaagaagtatcaattctgg

SEQ ID NO: 196 - DNA, 25 bp, Artificial
  Synthetic DNA
tcatctatcgaatttcctagcgatg

SEQ ID NO: 197 - DNA, 23 bp, Artificial
  Synthetic DNA
atgttgcaaacgaaaccgtacac

SEQ ID NO: 198 - DNA, 22 bp, Artificial
  Synthetic DNA
tcacttgaataaccttctgaag

SEQ ID NO: 199 - DNA, 22 bp, Artificial
  Synthetic DNA
atgttggaaaacactcaaaagg

SEQ ID NO: 200 - DNA, 22 bp, Artificial
  Synthetic DNA
tcaaccaaggttgatgtacgtc

SEQ ID NO: 201 - DNA, 22 bp, Artificial
  Synthetic DNA
atgcttaatacggaaaactacg

SEQ ID NO: 202 - DNA, 22 bp, Artificial
  Synthetic DNA
tcatttgatattcacgtacgtc

SEQ ID NO: 203 - DNA, 22 bp, Artificial
  Synthetic DNA
atgttgcaggtaaaagaatatg

SEQ ID NO: 204 - DNA, 22 bp, Artificial
  Synthetic DNA
tcaacaaatttccactagttcc

SEQ ID NO: 205 - DNA, 24 bp, Artificial
  Synthetic DNA
atgcttaagacaaatgactacaag

SEQ ID NO: 206 - DNA, 25 bp, Artificial
  Synthetic DNA
tcactcggaatcaacagcgaaagtc

SEQ ID NO: 207 - DNA, 25 bp, Artificial
  Synthetic DNA
gcggattgagagcaaatcgttaagt

SEQ ID NO: 208 - DNA, 37 bp, Artificial
  Synthetic DNA
ctatacagcggaattagaggcatagcggcaaactaag

SEQ ID NO: 209 - DNA, 48 bp, Artificial; identical to SEQ ID NO: 130
  Synthetic DNA

SEQ ID NO: 210 - DNA, 49 bp, Artificial
  Synthetic DNA
ataatgtatgctatacgaacggtaagggaaagatatgagctatacagcg

SEQ ID NO: 211 - DNA, 24 bp, Artificial
  Synthetic DNA
cccacacaccatagcttcaaaatg

SEQ ID NO: 212 - DNA, 35 bp, Artificial
  Synthetic DNA
atcaccgaaatcttcatgtttagttcctcaccttg

SEQ ID NO: 213 - DNA, 35 bp, Artificial
  Synthetic DNA
caaggtgaggaactaaacatgaagatttcggtgat

SEQ ID NO: 214 - DNA, 20 bp, Artificial
  Synthetic DNA
ttaggcgtcatcctgtgctc

SEQ ID NO: 215 - DNA, 45 bp, Artificial
  Synthetic DNA
caggatgacgcctaaaaagattctctttttttatgatatttgtac

SEQ ID NO: 216 - DNA, 30 bp, Artificial
  Synthetic DNA
aggaatcatagtttcatgattttctgttac

SEQ ID NO: 217 - DNA, 33 bp, Artificial
  Synthetic DNA
gaaactatgattcctacggattagaagccgccg

SEQ ID NO: 218 - DNA, 29 bp, Artificial
  Synthetic DNA
ggttttttctccttgacgttaaagtatag

SEQ ID NO: 219 - DNA, 37 bp, Artificial; identical to SEQ ID NO: 122
  Synthetic DNA

SEQ ID NO: 220 - DNA, 38 bp, Artificial; identical to SEQ ID NO: 121
  Synthetic DNA

SEQ ID NO: 221 - DNA, 26 bp, Artificial
  Synthetic DNA
ttagttatgtcacgcttacattcacg

SEQ ID NO: 222 - DNA, 22 bp, Artificial
  Synthetic DNA
agcttgcaaattaaagccttcg

SEQ ID NO: 223 - DNA, 49 bp, Artificial; identical to SEQ ID NO: 118
  Synthetic DNA

SEQ ID NO: 224 - DNA, 64 bp, Artificial; identical to SEQ ID NO: 117
  Synthetic DNA

SEQ ID NO: 225 - DNA, 36 bp, Artificial
  Synthetic DNA
tgttatagagttcacaccttattcacatactttttc

SEQ ID NO: 226 - DNA, 42 bp, Artificial
  Synthetic DNA
gtgaactctataacagtatgctgcagctttaaataatcggtg

SEQ ID NO: 227 - DNA, 23 bp, Artificial
  Synthetic DNA
aagggcgaattctgcagatatcc

SEQ ID NO: 228 - DNA, 22 bp, Artificial
  Synthetic DNA
aagggcgaattccagcacactg

SEQ ID NO: 229 - DNA, 29 bp, Artificial
  Synthetic DNA
tggctacagaatcataagttgaattcgac

SEQ ID NO: 230 - DNA, 46 bp, Artificial
  Synthetic DNA
gacccgtggctgcgagttttttctccttgacgttaaagtatagagg

SEQ ID NO: 231 - DNA, 18 bp, Artificial
  Synthetic DNA
tcgcagccacgggtcaac

SEQ ID NO: 232 - DNA, 53 bp, Artificial
  Synthetic DNA
ctcctcaactgccattttttattattagtcttttttttttttgacaatatctg

SEQ ID NO: 233 - DNA, 24 bp, Artificial
  Synthetic DNA
atggcagttgaggagaacaatatg

SEQ ID NO: 234 - DNA, 25 bp, Artificial
  Synthetic DNA
ttattctagcatggccttgtaccac

SEQ ID NO: 235 - DNA, 43 bp, Artificial; identical to SEQ ID NO: 77
  Synthetic DNA

SEQ ID NO: 236 - DNA, 34 bp, Artificial
  Synthetic DNA
ctatacagcggaattttactccgcaacgcttttc

SEQ ID NO: 237 - DNA, 39 bp, Artificial
  Synthetic DNA
attatacgaagttatacgacatcgtcgaatatgattcag

SEQ ID NO: 238 - DNA, 49 bp, Artificial; identical to SEQ ID NO: 210
  Synthetic DNA

SEQ ID NO: 239 - DNA, 24 bp, Artificial
  Synthetic DNA
acgacatcgtcgaatatgattcag

SEQ ID NO: 240 - DNA, 33 bp, Artificial; identical to SEQ ID NO: 82
  Synthetic DNA

SEQ ID NO: 241 - DNA, 36 bp, Artificial; identical to SEQ ID NO: 83
  Synthetic DNA

SEQ ID NO: 242 - DNA, 33 bp, Artificial; identical to SEQ ID NO: 84
  Synthetic DNA

SEQ ID NO: 243 - DNA, 37 bp, Artificial; identical to SEQ ID NO: 85
  Synthetic DNA

SEQ ID NO: 244 - DNA, 26 bp, Artificial
  Synthetic DNA
gggtaataactgatataattaaattg

SEQ ID NO: 245 - DNA, 37 bp, Artificial; identical to SEQ ID NO: 87
  Synthetic DNA

SEQ ID NO: 246 - DNA, 54 bp, Artificial; identical to SEQ ID NO: 88
  Synthetic DNA

SEQ ID NO: 247 - DNA, 31 bp, Artificial
  Synthetic DNA
ctactcataactttagcatcacaaaatacgc

SEQ ID NO: 248 - DNA, 36 bp, Artificial
  Synthetic DNA
gtgaaattaagaaaggagttttatacagatgatacc

SEQ ID NO: 249 - DNA, 22 bp, Artificial
  Synthetic DNA
acccggggatcctctagagtcg

SEQ ID NO: 250 - DNA, 23 bp, Artificial
  Synthetic DNA
gaattcactggccgtcgttttac

SEQ ID NO: 251 - DNA, 29 bp, Artificial
  Synthetic DNA
tggctacagaatcataagttgaattcgac

SEQ ID NO: 252 - DNA, 46 bp, Artificial; identical to SEQ ID NO: 230
  Synthetic DNA

SEQ ID NO: 253 - DNA, 18 bp, Artificial
  Synthetic DNA
tcgcagccacgggtcaac

SEQ ID NO: 254 - DNA, 53 bp, Artificial; identical to SEQ ID NO: 232
  Synthetic DNA

SEQ ID NO: 255 - DNA, 39 bp, Artificial; identical to SEQ ID NO: 185
  Synthetic DNA

SEQ ID NO: 256 - DNA, 42 bp, Artificial; identical to SEQ ID NO: 186
  Synthetic DNA

SEQ ID NO: 257 - DNA, 48 bp, Artificial
  Synthetic DNA
aatacatattcaaaatggacatgaccgccgcggaaatagtcagaaatg

SEQ ID NO: 258 - DNA, 36 bp, Artificial
  Synthetic DNA
ttctaacaaaacttctagccgttgtaagtcaacgcg

SEQ ID NO: 259 - DNA, 25 bp, Artificial
  Synthetic DNA
gcggattgagagcaaatcgttaagt

SEQ ID NO: 260 - DNA, 37 bp, Artificial; identical to SEQ ID NO: 208
  Synthetic DNA

SEQ ID NO: 261 - DNA, 43 bp, Artificial; identical to SEQ ID NO: 77
  Synthetic DNA

SEQ ID NO: 262 - DNA, 34 bp, Artificial; identical to SEQ ID NO: 236
  Synthetic DNA

SEQ ID NO: 263 - DNA, 44 bp, Artificial
  Synthetic DNA
ttctaacaaaacttcttatttctgaccgtaataggcattcttac

SEQ ID NO: 264 - DNA, 42 bp, Artificial
  Synthetic DNA
taatacatattcaaaatgttggagcagttaaaggaagaagtc

SEQ ID NO: 265 - DNA, 26 bp, Artificial
  Synthetic DNA
tagcgttgaatgttagcgtcaacaac

SEQ ID NO: 266 - DNA, 30 bp, Artificial; identical to SEQ ID NO: 140
  Synthetic DNA

SEQ ID NO: 267 - DNA, 64 bp, Artificial; identical to SEQ ID NO: 117
  Synthetic DNA

SEQ ID NO: 268 - DNA, 49 bp, Artificial; identical to SEQ ID NO: 118
  Synthetic DNA

SEQ ID NO: 269 - DNA, 26 bp, Artificial
  Synthetic DNA
ttagttatgtcacgcttacattcacg

SEQ ID NO: 270 - DNA, 22 bp, Artificial
  Synthetic DNA
agcttgcaaattaaagccttcg

SEQ ID NO: 271 - DNA, 38 bp, Artificial; identical to SEQ ID NO: 121
  Synthetic DNA

SEQ ID NO: 272 - DNA, 37 bp, Artificial; identical to SEQ ID NO: 122
  Synthetic DNA

SEQ ID NO: 273 - DNA, 29 bp, Artificial
  Synthetic DNA
ggttttttctccttgacgttaaagtatag

SEQ ID NO: 274 - DNA, 76 bp, Artificial; identical to SEQ ID NO: 124
  Synthetic DNA

SEQ ID NO: 275 - DNA, 24 bp, Artificial
  Synthetic DNA
cccacacaccatagcttcaaaatg

SEQ ID NO: 276 - DNA, 33 bp, Artificial
  Synthetic DNA
gtttagttcctcaccttgtcgtattatactatg

SEQ ID NO: 277 - DNA, 22 bp, Artificial
  Synthetic DNA
atgaagatttcggtgatccctg

SEQ ID NO: 278 - DNA, 20 bp, Artificial
  Synthetic DNA
ttaggcgtcatcctgtgctc

SEQ ID NO: 279 - DNA, 46 bp, Artificial
  Synthetic DNA
aaagattctctttttttatgatatttgtacataaactttataaatg

SEQ ID NO: 280 - DNA, 29 bp, Artificial
  Synthetic DNA
ggaatcatagtttcatgattttctgttac

SEQ ID NO: 281 - DNA, 26 bp, Artificial
  Synthetic DNA
tacgaacggtaagggaaagatatgag

SEQ ID NO: 282 - DNA, 48 bp, Artificial; identical to SEQ ID NO: 130
  Synthetic DNA

SEQ ID NO: 283 - DNA, 31 bp, Artificial; identical to SEQ ID NO: 247
  Synthetic DNA

SEQ ID NO: 284 - DNA, 36 bp, Artificial; identical to SEQ ID NO: 248
  Synthetic DNA

SEQ ID NO: 285 - DNA, 22 bp, Artificial
  Synthetic DNA
acccggggatcctctagagtcg

SEQ ID NO: 286 - DNA, 23 bp, Artificial
  Synthetic DNA
gaattcactggccgtcgttttac